(19) United States
(12) Patent Application Publication
(10) Pub. No.: US 2011/0136145 A1
(43) Pub. Date: Jun. 9, 2011

(54) METHODS FOR PROMOTING FUSION AND REPROGRAMMING OF SOMATIC CELLS

(75) Inventors: Hongjun Song, Clarksville, MD (US); Guo-Ii Ming, Clarksville, MD (US); Dengke K. Ma, Cambridge, MA (US)

(73) Assignee: THE JOHNS HOPKINS UNIVERSITY, Baltimore, MD (US)

(21) Appl. No.: 12/994,040

(22) PCT Filed: May 22, 2009
(86) PCT No.: PCT/US09/44971

§ 371 Date: Feb. 21, 2011

Related U.S. Application Data
(60) Provisional application No. 61/128,535, filed on May 22, 2008.

(51) Int. Cl. G01N 33/567; C12N 15/01; C12Q 1/02

(52) U.S. Cl. 435/7.21; 435/441; 435/29

(57) ABSTRACT

The invention features methods for reprogramming somatic cells by treating the cells with one or more agents to induce de-differentiation, in particular by targeting demethylase and methyltransferase genes. The invention also features methods of monitoring somatic cell fusion and reprogramming and methods of identifying agents that alter somatic cell fusion and reprogramming. The invention also features reprogrammed cells and kits.

### RELATED APPLICATIONS/PATENTS & INCORPORATION BY REFERENCE

This application claims the benefit of U.S. Provisional Application No. 61/128,535, filed on May 22, 2008. The entire contents of the aforementioned application are hereby incorporated herein by reference.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. More generally, documents or references are cited in this text, either in a Reference List before the claims, or in the text itself; and, each of these documents or references (“herein-cited references”), as well as each document or reference cited in each of the herein-cited references (including any manufacturer's specifications, instructions, etc.), is hereby expressly incorporated herein by reference.

### BACKGROUND OF THE INVENTION

Pluripotent stem cells have the potential to differentiate into the full range of daughter cells having distinctly different morphological, cytological or functional phenotypes unique to a specific tissue. By contrast, descendants of pluripotent cells are restricted progressively in their differentiation potential. Pluripotent cells have therapeutic potential, as they can be differentiated along the desired pathway in a precisely controlled manner and used in cell-based therapy and for agent screening, in particular for therapeutic agents.

Highly differentiated somatic nuclei, of both mice and humans, can be converted into a pluripotent state by methods including somatic cell nuclear transfer (SCNT), embryonic stem cell (ESC) fusion-mediated reprogramming, or by introducing defined genetic factors. Accumulating evidence suggests that oocyte cytoplasm, ESCs and early embryos are enriched in reprogramming factors, which function to erase the somatic epigenome and re-establish a pluripotent signature of gene expression. However, the molecular identities of these reprogramming factors and direct cellular mechanisms by which those factors work on somatic genomes are still not completely understood.

Oct4 encodes a member of POU (Pit-Oct-Unc) family of transcription factors that has been widely used as a specific marker for pluripotent ESCs. During early development, Oct4 is mainly expressed in the inner cell mass of blastocyst, and becomes down-regulated during cell differentiation. In somatic cells, Oct4 expression is repressed by epigenetic mechanisms involving both histone and DNA methylation to ensure silencing of Oct4 in a heritable manner. Consistent with its essential role for establishing pluripotency, both SCNT and ESC-mediated reprogramming induce re-activation of Oct4 from somatic genomes. The extent of Oct4 re-activation is directly related to the developmental potential of somatic cell clones, and incomplete re-activation contributes to the low efficiency of somatic reprogramming. While the tight regulation of Oct4 attests to its utility as a reliable marker for successful reprogramming, specific mechanisms of how reprogramming activities induces genome-wide changes, including somatic Oct4 re-activation, remain to be identified.

Most of the current reprogramming regimes using ESCs typically involve polyethylene glycol (PEG)-induced cell fusion of ESCs and somatic cells carrying two different drug resistant genes, followed by long-term selection to yield hybrid clones. The low frequency of cell fusion makes it challenging to immediately identify cells that have undergone fusion. As a consequence, very little is known about the essential process of reprogramming at the early stage. Double drug selection also leads to cell death and release of various factors, which may affect the reprogramming process.

Accordingly, a need remains for more effective and reliable methods of reprogramming. A better understanding of the process of reprogramming and de-differentiation will shed light on new targets and methods for somatic cell reprogramming.

### SUMMARY OF THE INVENTION

The present invention describes the development of a double fluorescent reporter system that, in preferred embodiments, uses engineered embryonic stem cells and somatic cells to simultaneously and independently monitor cell fusion and reprogramming-induced re-activation of GFP expression. In preferred embodiments, the present invention features methods wherein inhibition of a histone methyltransferase or over-expression of a histone demethylase promotes ESC fusion-induced GFP re-activation from somatic cells. In addition, in certain preferred embodiments of the invention, co-expression of Nanog and Jhdm2a further enhances the ESC-induced Oct4-GFP re-activation. These mechanistic findings may guide a more efficient reprogramming regime for future therapeutic applications of stem cells.

Accordingly, in a first aspect, the invention features a method for reprogramming one or more somatic cells comprising treating the cells with one or more agents that induces de-differentiation, wherein the agent is selected from a histone methyltransferase inhibitor or a histone demethylase activator, thereby generating a reprogrammed cell.

In another aspect, the invention features a method for reprogramming one or more somatic cells comprising treating the cells with one or more agents that induces de-differentiation, and detecting the expression of one or more markers, where at least one marker indicates cell reprogramming, selecting a cell that expresses the one or more markers, and thereby generating a reprogrammed cell.

In one embodiment, the method further comprises contacting a somatic cell with an embryonic stem cell.

In another aspect, the invention features a method for reprogramming one or more somatic cells comprising contacting a somatic cell with an embryonic stem cell, treating the cells with one or more agents that induces de-differentiation, detecting the expression of one or more markers, where at least one marker indicates cell reprogramming, selecting a cell that expresses the one or more markers, thereby generating a reprogrammed cell.

In another embodiment of any one of the above aspects, the somatic cell comprises a Cre recombinase protein.

In a further embodiment of the above aspects, the embryonic cell comprises a fluorescent Cre recombination excision reporter, and wherein detection of the fluorescent Cre recombination reporter is used to monitor cell fusion. In another related embodiment, the somatic cell further comprises GFP and detection of GFP is used to identify an agent that alters somatic cell reprogramming.

In another embodiment of the above aspects, the cells are contacted in the presence of polyethylene glycol (PEG).

In a further embodiment of the above aspects, the somatic cell is an adult neural stem cell (NSC).

In still another further embodiment of the above aspects, the somatic cell comprises an Oct4 gene that directs GFP activation. In a further related embodiment, the somatic cells are obtained from Oct4-GFP transgenic mice.

In another embodiment of the above aspects, the somatic cell has been engineered to stably co-express Cre and the puromycin resistance gene.

In still another embodiment of the above aspects, the embryonic cell comprises CAG-loxP-LacZ::neomycin-polyA-loxP-DsRed.T3 as the fluorescent Cre recombination excision reporter.

In an embodiment of the above aspects, the agent is selected from the group consisting of: a small molecule, a peptide and an oligonucleotide. In a related embodiment, the oligonucleotide is an inhibitory oligonucleotide selected from the group consisting of: a small inhibitory RNA (siRNA), a short hairpin RNA (shRNA), a microrna, an antisense, and a ribozyme.

In a related embodiment of the above aspects, the agent is selected from the group consisting of histone methyltransferase, histone acetyltransferase, histone deactylase, and histone demethylase inhibitors.

In still another related embodiment of the above aspects, the agent is selected from the group consisting of: histone methyltransferase, histone acetyltransferase, histone deactylase, and histone demethylase activators.

In another related embodiment of the above aspects, the agent modifies epigenetic histone methylation or demethylation.

In preferred embodiments of the above aspects, the reprogramming factor is a histone demethylase, for example any one or more of the following:

AOF (LSD1), AOF1 (LSD2), FBXL11 (JHDM1A), Fbxl10 (JHDM1B), FBXL19 (JHDM1C), KIAA1718 (JHDM1D), PHF2 (JHDM1E), PHF8 (JHDM1F), JMJD1A (JHDM2A), JMJD1B (JHDM2B), JMJD1C (JHDM2C), JMJD2A (JHDM3A), JMJD2B (JHDM3B), JMJD2C (JHDM3C), JMJD2D (JHDM3D), RBP2 (JARID1A), PLU1 (JARID1B), SMCX (JARID1C), SMCY (JARID1D), Jumonji (JARID2), UTX (UTX), UTY (UTY), JMJD3 (JMJD3), JMJD4 (JMJD4), JMJD5 (JMJD5), JMJD6 (JMJD6), JMJD7 (JMJD7), JMJD8 (JMJD8).

In further preferred embodiments of the above aspects, the histone demethylase is Jhdm2a.

In other embodiments of the above aspects, the reprogramming factor is an inhibitory oligonucleotide targeting a histone methyltransferase, example any one or more of the following:

SUV39H1, SUV39H2, G9A (EHMT2), EHMT1, ESET (SETDB1), SETDB2, MLL, MLL2, MLL3, SETD2, NSD1, SMYD2, DOT1L, SETD8, SUV420H1, SUV420H2, EZH2, SETD7, PRDM2, PRMT1, PRMT2, PRMT3, PRMT4, PRMT5, PRMT6, PRMT7, PRMT8, PRMT9, PRMT10, PRMT11, CARM1.

In other preferred embodiments of the above aspects, the histone methyltransferase is G9A.

In one particular embodiment of the above aspects, the agent is a Nanog activator.

In another particular embodiment of the above aspects, the inhibitor is a histone methyltransferase G9A inhibitor.

In another particular embodiment of the above aspects, the activator is a histone demethylase Jhdm2a activator.

In another embodiment of the above aspects, the treatment with one or more agents comprises transfecting the cells with a vector comprising at least one gene.

In a further embodiment of the above aspects, the gene is selected from a histone demethylase or a histone methyltransferase.

In preferred embodiments of the above aspects, the histone demethylase is selected from for example any one or more of the following:

AOF (LSD1), AOF1 (LSD2), FBXL11 (JHDM1A), Fbxl10 (JHDM1B), FBXL19 (JHDM1C), KIAA1718 (JHDM1D), PHF2 (JHDM1E), PHF8 (JHDM1F), JMJD1A (JHDM2A), JMJD1B (JHDM2B), JMJD1C (JHDM2C), JMJD2A (JHDM3A), JMJD2B (JHDM3B), JMJD2C (JHDM3C), JMJD2D (JHDM3D), RBP2 (JARID1A), PLU1 (JARID1B), SMCX (JARID1C), SMCY (JARID1D), Jumonji (JARID2), UTX (UTX), UTY (UTY), JMJD3 (JMJD3), JMJD4 (JMJD4), JMJD5 (JMJD5), JMJD6 (JMJD6), JMJD7 (JMJD7), JMJD8 (JMJD8).

In certain embodiments of the above aspects, the histone demethylase is Jhdm2a.

In other embodiments of the above aspects, histone methyltransferase is selected from, example any one or more of the following:

SUV39H1, SUV39H2, G9A (EHMT2), EHMT1, ESET (SETDB1), SETDB2, MLL, MLL2, MLL3, SETD2, NSD1, SMYD2, DOT1L, SETD8, SUV420H1, SUV420H2, EZH2, SETD7, PRDM2, PRMT1, PRMT2, PRMT3, PRMT4, PRMT5, PRMT6, PRMT7, PRMT8, PRMT9, PRMT10, PRMT11, CARM1.

In other embodiments of the above aspects, the histone methyltransferase is G9A.

In a particular embodiment of the above aspects, the genes are selected from the group consisting of: Jdhm2a, G9A and Nanog. In one particular embodiment of the above aspects, Jdhm2a corresponds to the nucleotide sequence set forth in SEQ ID NO: 5 or SEQ ID NO: 7. In one particular embodiment of the above aspects, G9A corresponds to the nucleotide sequence set forth in SEQ ID NO: 1 or SEQ ID NO: 3. In another particular embodiment of the above aspects, Nanog corresponds to the nucleotide sequence set forth in SEQ ID NO: 9 or SEQ ID NO: 11.

In another embodiment, the invention features a reprogrammed cell produced by the method of any one of the above aspects.

In still another embodiment, the invention features a reprogrammed cell obtained by the method of any one of the above aspects.

In one embodiment of any one of the above aspects, the somatic cell is a mammalian cell.

In another aspect, the invention features a kit comprising a reprogrammed somatic cell produced according to the methods of any one of the above aspects, and instructions for use.

In another aspect, the invention features a method of monitoring somatic cell fusion comprising contacting a somatic cell comprising a Cre recombinase protein with an embryonic cell, wherein the embryonic cell comprises a fluorescent Cre recombination excision reporter, and wherein detection of the fluorescent Cre recombination reporter is used to monitor cell fusion.

In one embodiment, the method further comprises the step of monitoring somatic cell reprogramming, wherein the somatic cell comprises GFP and detection of GFP is used to monitor reprogramming.

In another further embodiment, the somatic cell is an adult neural stem cell (NSC).

In a related embodiment, the somatic cell comprises an Oct4 transgene that directs GFP activation. In another further embodiment, the somatic cells are obtained from Oct4-GFP transgenic mice.

In another embodiment, the somatic cell has been engineered to stably co-express Cre and the puromycin resistance gene.

In one embodiment, the embryonic cell comprises CAG-loxP-LacZ::neomycin-polyA-loxP-DsRed.T3 as the fluorescent Cre recombination excision reporter.

In another aspect, the invention features a method of monitoring somatic cell fusion and reprogramming comprising contacting a somatic cell comprising an Oct4-GFP Cre recombinase protein with an embryonic cell, wherein the embryonic cell comprises a fluorescent Cre recombination excision reporter, and wherein detection of the fluorescent Cre recombination reporter is used to monitor cell fusion and detection of GFP is used to monitor reprogramming.

In one embodiment of the above aspects, fusion or reprogramming are monitored using fluorescent microscopy or flow cytometry.

In one embodiment, dual-color flow cytometry is used to quantitatively monitor cell fusion.

In another further embodiment, flow cytometry is used to monitor reprogramming frequency, wherein reprogramming frequency is represented by the ratio of GFP+DsRed+ cells to total DsRed+ cells.

In still another embodiment, flow cytometry is used to monitor reprogramming efficacy, wherein reprogramming efficacy is represented by the distribution of GFP fluorescence intensity of individual cells from the DsRed+population.

In another embodiment, the method provides a measurement of the efficacy of Oct4-GFP reactivation in somatic cells after fusion.

In another aspect, the invention provides a method of identifying an agent that alters somatic cell fusion comprising contacting a somatic cell comprising a Cre recombinase protein with an embryonic cell, wherein the embryonic cell comprises a fluorescent Cre recombination excision reporter, and wherein detection of the fluorescent Cre recombination reporter is used to monitor cell fusion; contacting the cells with a candidate agent, wherein detection of the fluorescent Cre recombination reporter is used to identify an agent that alters somatic cell fusion.

In one embodiment, the method further comprises identifying an agent that alters somatic cell reprogramming comprising the step of monitoring somatic cell reprogramming, wherein the somatic cell comprises GFP and detection of GFP is used to identify an agent that alters somatic cell reprogramming.

In one embodiment, the cells are contacted with the candidate agent 24-48 hours after cell fusion.

In another embodiment of any one of the above aspects, the cells are contacted in the presence of polyethylene glycol (PEG).

In another further embodiment, the somatic cell is an adult neural stem cell (NSC).

In a related embodiment, the somatic cell comprises an Oct4 transgene that directs GFP activation. In another further embodiment, the somatic cells are obtained from Oct4-GFP transgenic mice.

In another embodiment, the somatic cell has been engineered to stably co-express Cre and the puromycin resistance gene.

In one embodiment, the embryonic cell comprises CAG-loxP-LacZ::neomycin-polyA-loxP-DsRed.T3 as the fluorescent Cre recombination excision reporter.

In another aspect, the invention features a method of identifying an agent that alters somatic cell fusion and reprogramming comprising contacting a somatic cell comprising a Oct4-GFP Cre recombinase protein with an embryonic cell, wherein the embryonic cell comprises a fluorescent Cre recombination excision reporter, and wherein detection of the fluorescent Cre recombination reporter is used to monitor cell fusion; contacting the cells with a candidate agent, wherein detection of the fluorescent Cre recombination reporter is used to identify an agent that alters somatic cell fusion and detection of GFP is used to identify an agent that alters somatic cell reprogramming.

In one embodiment of any one of the above aspects, fusion or reprogramming are monitored using fluorescent microscopy or flow cytometry.

In another embodiment, dual-color flow cytometry is used to quantitatively monitor cell fusion.

In still another embodiment, flow cytometry is used to monitor reprogramming frequency, wherein reprogramming frequency is represented by the ratio of GFP+DsRed+ cells to total DsRed+ cells. In a further embodiment, reprogramming frequency is monitored after treatment with the agent. In another related embodiment, flow cytometry is used to monitor reprogramming efficacy, wherein reprogramming efficacy is represented by the distribution of GFP fluorescence intensity of individual cells from the DsRed+ population.

In another embodiment, the method provides a measurement of the efficacy of Oct4-GFP reactivation in somatic cells after fusion. In a further embodiment, the reprogramming efficacy is monitored after treatment with the agent.

In one embodiment, the agent is selected from the group consisting of: small molecules, peptides and oligonucleotides. In a further embodiment, the agent is a histone demethylase inhibitor. In one embodiment, histone demethylase is selected from the group consisting of AOF (LSD1), AOF1 (LSD2), FBXL11 (JHDM1A), Fbxl10 (JHDM1B), FBXL19 (JHDM1C), KIAA1718 (JHDM1D), PHF2 (JHDM1E), PHF8 (JHDM1F), JMJD1A (JHDM2A), JMJD1B (JHDM2B), JMJD1C (JHDM2C), JMJD2A (JHDM3A), JMJD2B (JHDM3B), JMJD2C (JHDM3C), JMJD2D (JHDM3D), RBP2 (JARID1A), PLU1 (JARID1B), SMCX (JARID1C), SMCY (JARID1D), Jumonji (JARID2), UTX (UTX), UTY (UTY), JMJD3 (JMJD3), JMJD4 (JMJD4), JMJD5 (JMJD5), JMJD6 (JMJD6), JMJD7 (JMJD7), JMJD8 (JMJD8). In certain preferred embodiments, the histone demethylase is Jhdm2a. In other preferred embodiment, the histone demethylase is a DNA repair demethylases and the jumani family of histone demethylases.

In one embodiment, the invention features a kit comprising a reprogrammed somatic cell produced according to any one of the methods of any one of the aspects herein, and instructions for use.

In another embodiment, the invention features a kit for monitoring somatic cell fusion comprising a somatic cell comprising a Cre recombinase protein and an embryonic cell comprising a fluorescent Cre recombination excision reporter, and instructions for use according to any of the methods of the aspects herein.

In another particular embodiment, the kit is used in monitoring cell reprogramming, along with instructions for use.

Other aspects of the invention are described in the following disclosure, and are within the scope of the invention.

### BRIEF DESCRIPTION OF THE DRAWINGS

The following Detailed Description, given by way of example, but not intended to limit the invention to specific embodiments described, may be understood in conjunction with the accompanying drawings, incorporated herein by reference. Various preferred features and embodiments of the present invention will now be described by way of non-limiting example and with reference to the accompanying drawings in which:

FIG. 1 (A-C) shows Cre-loxP-based, EG-FP-inducible Assay for Reprogramming (CLEAR). (A) is a diagrammatic illustration of CLEAR analysis. CIPOE NSC lines were established by infection of adult NSCs derived from transgenic mice harboring Oct4-GFP reporter with retroviruses to co-express the Cre recombinase and puromycin resistance gene. Z-Red ESCs carry an inducible DsRed expression cassette upon Cre mediated excision. PEG-induced fusion of Z-Red ESC and CIPOE NSCs leads to GFP expression as an indicator for Oct4 reactivation and DsRed expression as a reporter for fusion events. The dual-color reporter system can then be monitored by both live fluorescence microscopy and quantitative flow cytometry to probe reprogramming processes. (B, C) are 1 images of fused ES-like colonies. Shown are sample images of fused ES-like colonies at 48 hours (B) and 96 hours (C) after PEG-induced fusion between CIPOE NSCs and Z-Red ESCs. An arrow points to DsRed GFP− cells that were successfully fused but incompletely reprogrammed. Scale bar: 20 μM.

FIGS. 2 (A and B) shows characterization of Z-Red ESCs and CIPOE NSCs. (A) shows immunocytochemical analysis of Z-Red ESCs transfected with a vector for constitutive Cre expression. Shown are DAPI (blue), anti-DsRed (red), anti-Oct4 (green) and merged images. Scale bar 50 μm (B) shows immunocytochemical analysis of CIPOE NSCs transfected with a Cre excision reporter plasmid pCAGT-bGeo-LoxP. Shown are images of staining for DsRed (red), DAPI (blue), Cre (green) and merged. Scale bar: 20 μm.

FIG. 3 (A-E) shows isolation and characterization of fused clones. (A) A GFP′ hybrid clone from PEG-induced cell fusion between ESCs and NSCs selected in the presence of neomycin and puromycin. Scale bars: 20 μm. (B) Expansion of the hybrid clone B3 under ESC culture conditions. (C) Formation of embryoid bodies from the clonal hybrid ESC line B3. (D) Quantitative PCR analysis of Oct4 expression in B3 and B3-derived embryoid bodies. Values represent mean±SEM (n=4, **. P<0.01, student t-test). (E) DNA (stained by propidium iodine) content analysis of the B3 hybrid clone (GFP±) compared with mixed wild type ESCs (GFP−).

FIG. 4 (A-C) shows flow cytometry analysis of Oct4-GFP reactivation using CLEAR. (A) Representative dot plots. Shown are sample plots from control cell population including CIPOE NSCs only; Z-Red ESCs only; Z-Red ESCs transfected with a constitutive Cre expression plasmid, CIPOE NSCs transfected with a Cre reporter plasmid (pCAGT-bGeo-LoxP), and mixture of Z-Red ESCs without PEG; and from PEG-induced fusion cell population at 2, 4, and 6 days in vitro (DIV). (B) Analysis of reprogramming frequency (Rf). Rf is calculated from multiple independent experiments and quantified for fusion population over 2, 4, 6, 8 days after PEG treatment. Values represent mean+SEM. (n=5; *P<0.01, One-Way ANOVA). (C) Analysis of reprogramming efficacy. Shown is the summary of cumulative distribution plot of GFP intensity for grouped DsRed+ cells over 2, 4, 6, 8 days after PEG treatment. Values represent mean+SEM. (n=5; * P<0 01, Kolmogorov-Smirnov test).

FIG. 5 (A-C) shows expression of DsRed does not change over time or by different experimental manipulations. (A) shows a time-course analysis of DsRed expression. Shown is cumulative distribution plot of DsRed′ population with graded DsRed′ fluorescence intensities over a period of 2, 4, 6, 8 days. Values represent mean±SEM (n=0.10, Kolmogorov-Smirnov test). (B) shows expression of DsRed with DMOG treatment. Shown is the cumulative distribution plot of DsRed+ population at day 4 with graded DsRed+ fluorescence intensities with treatment of DMSO or 10 [Al DMOG. Values represent mean±SEM (n=3; P 0.10, Kolmogorov-Smirnov test). (C) Comparison of the reprogramming efficacy of DsRed cells with the total cell population. Shown is the cumulative distribution plot of GFP+DsRed+, GFP−DsRed−, or GFP+ cell population at day 8 after cell fusion. Values represent mean±SEM (n=3; P>0.10, Kolmogorov-Smirnov test).

FIG. 6 (A-D) shows dioxygenase inhibitor DMOG impedes reprogramming. (A) Inhibition of Jhdm2a induced histone demethylation by DMOG. Blocking effects of DMOG on Jhdm2a were examined in 293T cells transfected with a plasmid expressing Jhdm2a-EGFP fusion protein. In control cells treated with DMSO, histone 3 lysine 9 dimethylation (H3K9diM) immunostaining signal was lost in Jhdm2a-GFP transfected cells (arrows). With the treatment of 10 μM DMOG, H3K9 dimethylation signals are present in all cells regardless of Jhdm2a GFP expression. DAPI labels all cell nuclei. Scale bar: 20 μm. (B-D) show DMOG attenuates ESC-induced reactivation of Oct4 expression from adult NSCs. PEG-mediated cell fusion population treated with DMSO or DMOG (10 μM) was analyzed at day 4 and displayed in dot plots. Representative plots from multiple experiments are shown (B). Reprogramming frequencies from multiple experiments were quantified (C) for fusion population with the 48-hour treatment of DMSO or DMOG. Data represent mean±SEM. (n=3; ** P<0.01, Student's t-test). Shown in (D) is the cumulative distribution plot of DsRed+ population with graded GFP fluorescence intensities for comparison of the reprogramming efficacy in the presence of DMSO or DMOG (n=3 experiments; * P<0.01, Kolmogorov-Smirnov test).

FIG. 7 (A-C) shows histone methyltransferase G9a restricts Oct4-GFP reactivation during ESC-induced reprogramming. (A) shows expression of G9a in ESCs and adult NSCs. Shown is a summary of quantitative real-time PCR analysis of the expression level of G9a in Z-Red ESCs, CIPOE NSCs, CIPOE NSCs with control shRNA or G9a-targeted shRNA. The mRNA abundance was normalized to the levels in CIPOE cells. Values represent mean+SEM (n=3; * P<0.01, Students t-test). (B) shows a summary of reprogramming frequencies. Shown are results from cell fusion experiments between Z-Red and CIPOE, CIPOE-shControl, or CIPOE-shG9a cells. Values represent mean+SEM (n=3; *- P<0.01 Students t-test). (C) Summary of reprogramming efficacy. Cumulative distribution plots of DsRed+ population with graded GFP fluorescence intensities are shown for comparison of the reprogramming efficacy of Z-Red ESCs with CIPOE-shControl, or CIPOE-shG9a cells at day 2 and day 4 after cell fusion. Values represent mean±SEM (n=3; *: P<0.01, Kolmogorov-Smirnov test).

FIG. 8 (A-E) shows histone demethylase Jhdm2a facilitates Oct4 reactivation during ESC-induced reprogramming. (A) shows EST profiles of histone demethylases. EST counts (transcripts per million) are collected from NCBI Unigene expression resources (available publicly on the world wide web at ncbi.nlm.nih.gov/sites/entrez?db=unigene) and plotted against a panel of currently identified histone demethylases. Distributions of EST for individual demethylases across different developmental stages are shown. (B) shows expression of Jhdm2a in ESCs and adult NSCs. Shown is quantitative real-time PCR analyses of the expression levels of Jhdm2a in Z-Red ESCs, CIPOE NSCs, CIPOE NSCs with virally transduced Jhdm2a wt or HI I20Y enzymatically inactive mutant. niRNA abundance is normalized to the levels of Z-Red cells and values represent mean±SEM. (n=3; * P<0.01, Student's t-test). (C) shows ecotopic expression of Jhdm2a, but not the enzyme-inactive mutant, induces cell-wide loss of H3K9 dimethylation in CIPOE NSCs as indicated by arrows. Scale bar 20 μm. (D) shows a summary of reprogramming frequencies. Shown are results from cell fusion experiments between Z-Red and CIPOE, CIPOE-J2WT, or CIPOE-J2HY cells. Values represent mean+SEM (n=3, *: P<0.01, Student's t-test). (E) is a summary of reprogramming efficacy. Cumulative distribution plots of DsRed+ population with graded GFP fluorescence intensities are shown for comparison of the reprogramming efficacy of Z-Red ESCs with CIPOE-J2, or CIPOE-J2HY cells at day 4 after cell fusion. Values represent mean±SEM (n=3, * P<0.01, Kolmogorov-Smirnov test).

FIG. 9 (A-C) shows synergistic enhancement of reprogramming by combination of Jlidin2a with the pluripotency-specific transcription factor Nanog. (A) is a schematic drawing of the model on mechanisms underlying ESC fusion-induced Oct4-GFP reactivation during reprogramming of somatic NSCs. (B) is a summary of reprogramming frequencies. Shown are results from cell fusion experiments between Z-Red and CIPOE, CIPOE-Nanog, or CIPOE-Nanog+Jhdm2a cells. Values represent mean±SEM (n=3; *. P<0.01, Student's t-test). (C) is a summary of reprogramming efficacy. Cumulative distribution plots of DsRed+ population with graded GFP fluorescence intensities are shown for comparison of the reprogramming efficacy of Z-Red ESCs with CIPOE, CIPOE-Jhdm2a or CIPOE-Nanog+Jhdm2a cells at day 4 after cell fusion. Values represent mean±SEM (n=3; *• P<0.01 Kolmogorov-Smirnov test).

FIG. 10 (A-C) shows Oct4 reactivation and promoter demethylation in adult NSCs after G9a knockdown. (A) is a schematic drawing of Oct4 promoter with 16 CpG sites, which are located between the proximal enhancer and exon 1. Bisulfite sequencing analysis is performed for genomic DNA extracted from CIPOE, Z-Red ESCs, fusion clone B3, CIPOE-shControl and CIPOE-shG9a NSCs. (B, C) show conventional RT-PCR (B) and quantitative real-time PCR(C) are used to compare the mRNA abundance of Oct4 in CIPOE, Z-Red ESCs, fusion clone B3, CIPOE-shControl and CIPOE-shG9a NSCs. Data represent mean+SEM. (n=3, *. P<0.01, Student's t-test).

### DETAILED DESCRIPTION OF THE INVENTION

### I. Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

As used in the specification and claims, the singular form “a”, “an” and “the” include plural references unless the context clearly dictates otherwise.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Unless specifically stated or obvious from context, as used herein, the term “or” is understood to be inclusive.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

In this disclosure, “comprises,” “comprising,” “containing” and “having” and the like can have the meaning ascribed to them in U.S. Patent law and can mean“includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments. The terms “administration” or “administering” are defined to include an act of providing a compound or pharmaceutical composition of the invention to a subject in need of treatment. In the instant invention, preferred routes of administration include parenteral administration, preferably, for example by injection, for example by intravenous injection.

By “agent” is understood herein to refer to a compound, for example a non-cell based compound, or a biologically active substance, including a gene, peptide or nucleic acid therapeutic, cytokine, antibody, etc. An agent can be a previously known or unknown compound.

By “cell fusion” is meant to refer to a process whereby membranes of two or more cells fuse. In preferred embodiment, cell fusion refers to direct intercellular sharing and interaction of cytoplasmic or nuclear contents.

By “de-differentiation” is meant to refer to a process whereby a cell changes from a more specialized function to a cell that has a less specialized function. Through the process of de-differentiation a cell can become pluripotent.

By “embryonic stem cell” is meant to refer to a cell that can grow indefinitely while maintaining pluripotency and can differentiate into cells of all three germ layers.

By “histone methyltransferase” is meant to refer to a family of enzymes, histone-lysine N-methyltransferase and histone-arginine N-methyltransferase, which catalyze the transfer of one to three methyl groups from the cofactor S-Adenosyl methionine to lysine and arginine residues of histone proteins. In preferred embodiments, the histone methyltransferase is G9A.

By “histone demethylase” is meant to refer to a family of enzymes that removes methyl groups appended to histone proteins that bind DNA and help regulate gene activity. In exemplary embodiments, the histone demethylase is Jdhm2a.

As used herein, “kits” are understood to contain at least the non-standard laboratory reagents of the invention and one or more non-standard laboratory reagents for use in the methods of the invention.

By “obtaining” is meant to refer to manufacturing, purchasing, or otherwise coming into possession of.

By “pluripotent cell” is meant a cell that has the potential to divide in vitro for a long period of time (e.g. greater than one year) and has the ability to differentiate into cells derived from all three embryonic germ layers—endoderm, mesoderm and ectoderm.

By “pluripotency gene”, as used herein, is meant to refer to a gene that is associated with pluripotency. The expression of a pluripotency gene is typically restricted to pluripotent stem cells, and is crucial for the functional identity of pluripotent stem cells. An example of a pluripotency gene is the transcription factor Oct-4.

By “reprogramming” is meant to refer to a process that alters or reverses the differentiation status of a somatic cell, where the somatic cell can be either partially or terminally differentiated. Reprogramming includes complete reversion, as well as partial reversion, of the differentiation status of a somatic cell.

By “reprogramming frequency” is meant to refer to a parameter for measuring the degree of reprogramming based on the percentage of reprogrammed cells among all fused cells. In certain embodiments, reprogramming frequency is represented by the ratio of GFP+DsRed+ cells to total DsRed+ cells.

By “reprogramming efficacy” is meant to refer to a parameter to measure the degree of reprogramming based on the expression level of reprogramming indicator proteins in fused cells. In certain embodiments, reprogramming efficacy is represented by the distribution of GFP fluorescence intensity of individual cells from the DsRed+ population.

By “somatic cell” is meant to refer to any cells except cells that maintain undifferentiated state and pluripotency. Exemplary somatic cells include, but are not limited to, tissue stem cells (somatic stem cells) such as neural stem cells, hematopoietic stem cells, mesenchymal stem cells, and spermatogonial stem cells, tissue progenitor cells, differentiated cells such as lymphocytes, epithelial cells, myocytes, and fibroblasts, and any cells that do not have an undifferentiated state and pluripotency.

By “stem cell” is meant to refer to a cell that can differentiate into many different cell types. Two broad types of mammalian stem cells are embryonic stem (ES) cells that are isolated from the inner cell mass of blastocysts, and adult stem cells that are found in adult tissues. In preferred embodiments, the stem cells are neural stem cells (NSC).

Other definitions appear in context throughout the disclosure.

### Methods of the Invention

The present invention describes the reprogramming of somatic cells by treating the cells with one or more agents that induce de-differentiation. The present invention also describes the development of a double fluorescent reporter system that, in preferred embodiments, uses engineered embryonic stem cells (ESCs) and adult neural stem cells (NSCs) to simultaneously and independently monitor cell fusion and reprogramming-induced re-activation of transgenic Oct4-GFP expression. In preferred embodiments, the present invention features methods where knockdown of a histone methyltransferase, for example G9A, or over-expression of a histone demethylase, for example Jhdm2a, promotes ESC fusion-induced Oct4-GFP re-activation from adult NSCs. In addition, in certain preferred embodiments of the invention, co-expression of Nanog and Jhdm2a further enhances the ESC-induced Oct4-GFP re-activation.

In certain embodiments, human G9A corresponds to the nucleotide sequence set forth by NCBI reference No. NM_{—}006709.3, shown below as SEQ ID NO: 1, and the corresponding amino acid sequence set forth by NCBI reference No. NP_{—}006700.3, shown below as SEQ ID NO: 2.

| SEQ ID NO: 1 | |
|---|---|
| 1 | gcaagcggcg atggcggcgg cggcgggagc tgcagcggcg gcggccgccg agggggaggc |
| 61 | ccccgctgag atgggggcgc tgctgctgga gaaggaaacc agaggagcca ccgagagagt |
| 121 | tcatggctct ttgggggaca cccctcgtag tgaagaaacc ctgcccaagg ccacccccga |
| 181 | ctccctggag cctgctggcc cctcatctcc agcctctgtc actgtcactg ttggtgatga |
| 241 | gggggctgac acccctgtag gggctacacc actcattggg gatgaatctg agaatcttga |
| 301 | gggagatggg gacctccgtg ggggccggat cctgctgggc catgccacaa agtcattccc |
| 361 | ctcttccccc agcaaggggg gttcctgtcc tagccgggcc aagatgtcaa tgacaggggc |
| 421 | gggaaaatca cctccatctg tccagagttt ggctatgagg ctactgagta tgccaggagc |
| 481 | ccagggagct gcagcagcag ggtctgaacc ccctccagcc accacgagcc cagagggaca |
| 541 | gcccaaggtc caccgagccc gcaaaaccat gtccaaacca ggaaatggac agcccccggt |
| 601 | ccctgagaag cggccccctg aaatacagca tttccgcatg agtgatgatg tccactcact |
| 661 | gggaaaggtg acctcagatc tggccaaaag gaggaagctg aactcaggag gtggcctgtc |
| 721 | agaggagtta ggttctgccc ggcgttcagg agaagtgacc ctgacgaaag gggaccccgg |
| 781 | gtccctggag gagtgggaga cggtggtggg tgatgacttc agtctctact atgattccta |
| 841 | ctctgtggat gagcgcgtgg actccgacag caagtctgaa gttgaagctc taactgaaca |
| 901 | actaagtgaa gaggaggagg aggaagagga ggaagaagaa gaagaggaag aggaggagga |
| 961 | agaggaagaa gaagaggaag atgaggagtc agggaatcag tcagatagga gtggttccag |
| 1021 | tggccggcgc aaggccaaga agaaatggcg aaaagacagc ccatgggtga agccgtctcg |
| 1081 | gaaacggcgc aagcgggagc ctccgcgggc caaggagcca cgaggagtga atggtgtggg |
| 1141 | ctcctcaggc cccagtgagt acatggaggt ccctctgggg tccctggagc tgcccagcga |
| 1201 | ggggaccctc tcccccaacc acgctggggt gtccaatgac acatcttcgc tggagacaga |
| 1261 | gcgagggttt gaggagttgc ccctgtgcag ctgccgcatg gaggcaccca agattgaccg |
| 1321 | catcagcgag agggcggggc acaagtgcat ggccactgag agtgtggacg gagagctgtc |
| 1381 | aggctgcaat gccgccatcc tcaagcggga gaccatgagg ccatccagcc gtgtggccct |
| 1441 | gatggtgctc tgtgagaccc accgcgcccg catggtcaaa caccactgct gcccgggctg |
| 1501 | cggctacttc tgcacggcgg gcaccttcct ggagtgccac cctgacttcc gtgtggccca |
| 1561 | ccgcttccac aaggcctgtg tgtctcagct gaatgggatg gtcttctgtc cccactgtgg |
| 1621 | ggaggatgct tctgaagctc aagaggtgac catcccccgg ggtgacgggg tgaccccacc |
| 1681 | ggccggcact gcagctcctg cacccccacc cctgtcccag gatgtccccg ggagagcaga |
| 1741 | cacttctcag cccagtgccc ggatgcgagg gcatggggaa ccccggcgcc cgccctgcga |
| 1801 | tcccctggct gacaccattg acagctcagg gccctccctg accctgccca atgggggctg |
| 1861 | cctttcagcc gtggggctgc cactggggcc aggccgggag gccctggaaa aggccctggt |
| 1921 | catccaggag tcagagaggc ggaagaagct ccgtttccac cctcggcagt tgtacctgtc |
| 1981 | cgtgaagcag ggcgagctgc agaaggtgat cctgatgctg ttggacaacc tggaccccaa |
| 2041 | cttccagagc gaccagcaga gcaagcgcac gcccctgcat gcagccgccc agaagggctc |
| 2101 | cgtggagatc tgccatgtgc tgctgcaggc tggagccaac ataaatgcag tggacaaaca |
| 2161 | gcagcggacg ccactgatgg aggccgtggt gaacaaccac ctggaggtag cccgttacat |
| 2221 | ggtgcagcgt ggtggctgtg tctatagcaa ggaggaggac ggttccacct gcctccacca |
| 2281 | cgcagccaaa atcgggaact tggagatggt cagcctgctg ctgagcacag gacaggtgga |
| 2341 | cgtcaacgcc caggacagtg gggggtggac gcccatcatc tgggctgcag agcacaagca |
| 2401 | catcgaggtg atccgcatgc tactgacgcg gggcgccgac gtcaccctca ctgacaacga |
| 2461 | ggagaacatc tgcctgcact gggcctcctt cacgggcagc gccgccatcg ccgaagtcct |
| 2521 | tctgaatgcg cgctgtgacc tccatgctgt caactaccat ggggacaccc ccctgcacat |
| 2581 | cgcagctcgg gagagctacc atgactgcgt gctgttattc ctgtcacgtg gggccaaccc |
| 2641 | tgagctgcgg aacaaagagg gggacacagc atgggacctg actcccgagc gctccgacgt |
| 2701 | gtggtttgcg cttcaactca accgcaagct ccgacttggg gtgggaaatc gggccatccg |
| 2761 | cacagagaag atcatctgcc gggacgtggc tcggggctat gagaacgtgc ccattccctg |
| 2821 | tgtcaacggt gtggatgggg agccctgccc tgaggattac aagtacatct cagagaactg |
| 2881 | cgagacgtcc accatgaaca tcgatcgcaa catcacccac ctgcagcact gcacgtgtgt |
| 2941 | ggacgactgc tctagctcca actgcctgtg cggccagctc agcatccggt gctggtatga |
| 3001 | caaggatggg cgattgctcc aggaatttaa caagattgag cctccgctga ttttcgagtg |
| 3061 | taaccaggcg tgctcatgct ggagaaactg caagaaccgg gtcgtacaga gtggcatcaa |
| 3121 | ggtgcggcta cagctctacc gaacagccaa gatgggctgg ggggtccgcg ccctgcagac |
| 3181 | catcccacag gggaccttca tctgcgagta tgtcggggag ctgatctctg atgctgaggc |
| 3241 | tgatgtgaga gaggatgatt cttacctctt cgacttagac aacaaggatg gagaggtgta |
| 3301 | ctgcatagat gcccgttact atggcaacat cagccgcttc atcaaccacc tgtgtgaccc |
| 3361 | caacatcatt cccgtccggg tcttcatgct gcaccaagac ctgcgatttc cacgcatcgc |
| 3421 | cttcttcagt tcccgagaca tccggactgg ggaggagcta gggtttgact atggcgaccg |
| 3481 | cttctgggac atcaaaagca aatatttcac ctgccaatgt ggctctgaga agtgcaagca |
| 3541 | ctcagccgaa gccattgccc tggagcagag ccgtctggcc cgcctggacc cacaccctga |
| 3601 | gctgctgccc gagctcggct ccctgccccc tgtcaacaca tgagaacgga ccacaccctc |
| 3661 | tctccccagc atggatggcc acagctcagc cgcctcctct gccaccagct gctcgcagcc |
| 3721 | catgcctggg ggtgctgcca tcttctctcc ccaccaccct ttcacacatt cctgaccaga |
| 3781 | gatcccagcc aggccctgga ggtctgacag cccctccctc ccagagctgg ttcctccctg |
| 3841 | ggagggcaac ttcagggctg gccacccccc gtgttcccca tcctcagttg aagtttgatg |
| 3901 | aattgaagtc gggcctctat gccaactggt tccttttgtt ctcaataaat gttgggtttg |
| 3961 | gtaataaaaa aaaaaaaaaa aa |
| SEQ ID NO: 2 | |
| 1 | maaaagaaaa aaaegeapae mgallleket rgatervhgs lgdtprseet 1pkatpdsle |
| 61 | pagpsspasv tvtvgdegad tpvgatplig desenlegdg dlrggrillg hatksfpssp |
| 121 | skggscpsra kmsmtgagks ppsvqslamr llsmpgaqga aaagsepppa ttspegqpkv |
| 181 | hrarktmskp gngqppvpek rppeiqhfrm sddvhslgkv tsdlakrrkl nsggglseel |
| 241 | gsarrsgevt ltkgdpgsle ewetvvgddf slyydsysvd ervdsdskse vealteqlse |
| 301 | eeeeeeeeee eeeeeeeeee eeedeesgnq sdrsgssgrr kakkkwrkds pwvkpsrkrr |
| 361 | krepprakep rgvngvgssg pseymevplg slelpsegtl spnhagvsnd tssletergf |
| 421 | eelplcscrm eapkidrise raghkcmate svdgelsgcn aailkretmr pssrvalmvl |
| 481 | cethrarmvk hhccpgcgyf ctagtflech pdfrvahrfh kacvsqlngm vfcphcgeda |
| 541 | seaqevtipr gdgvtppagt aapappplsq dvpgradtsq psarmrghge prrppcdpla |
| 601 | dtidssgpsl tlpnggclsa vglplgpgre alekalviqe serrkklrfh prqlylsvkq |
| 661 | gelqkvilml ldnldpnfqs dqqskrtplh aaaqkgsvei chvllqagan inavdkqqrt |
| 721 | plmeavvnnh levarymvqr ggcvyskeed gstclhhaak ignlemvsll lstgqvdvna |
| 781 | qdsggwtpii waaehkhiev irmlltrgad vtltdneeni clhwasftgs aaiaevllna |
| 841 | rcdlhavnyh gdtplhiaar esyhdcvllf lsrganpelr nkegdtawdl tpersdvwfa |
| 901 | 1q1nrklrlg vgnrairtek iicrdvargy envpipcvng vdgepcpedy kyisencets |
| 961 | tmnidrnith lqhctcvddc sssncicgql sircwydkdg rllqefnkie pplifecnqa |
| 1021 | cscwrncknr vvqsgikvrl qlyrtakmgw gvralqtipq gtficeyvge lisdaeadvr |
| 1081 | eddsylfdld nkdgevycid aryygnisrf inhlcdpnii pvrvfmlhqd lrfpriaffs |
| 1141 | srdirtgeel gfdygdrfwd ikskyftcqc gsekckhsae aialeqsrla rldphpellp |
| 1201 | elgslppvnt |

In certain embodiments, mouse G9A corresponds to the nucleotide sequence set forth by NCBI reference No. NM_{—}145830.1, shown below as SEQ ID NO: 3, and the corresponding amino acid sequence set forth by NCBI reference No. NP_{—}665829.1, shown below as SEQ ID NO: 4.

| SEQ ID NO: 3 | |
|---|---|
| 1 | atgcggggtc tgccgagagg gagggggctg atgcgggccc gggggcgggg gcgtgcggcc |
| 61 | cccacgggcg gccgcggccg cggtcggggg ggcgcccacc gagggcgagg taggccccga |
| 121 | agcctgctct cgctgcccag ggcccaggcg tcttgggccc cccagctgcc tgccgggctg |
| 181 | accggccccc cggttccttg tctcccctcc cagggggagg cccccgctga gatgggggcg |
| 241 | ctgctgctgg agaaggagcc ccgaggagcc gccgagagag ttcatagctc tttgggggac |
| 301 | acccctcaga gtgaggagac ccttcccaag gccaaccccg actccttgga gcctgccggc |
| 361 | ccctcctctc cggcctctgt cactgtcacc gtcggcgatg agggggctga cacccctgtc |
| 421 | ggggccgcat cactcatcgg ggacgaaccc gagagcctgg agggagatgg gggtcgcatc |
| 481 | gtgctgggcc atgccacaaa gtcgttcccc tcttccccca gcaagggggg tgcctgtccc |
| 541 | agtcgggcca aaatgtcaat gacaggggca ggaaagtcgc ccccctcggt ccagagtttg |
| 601 | gccatgaggc tgttgagcat gcccggggcc cagggagctg caactgctgg gcctgaaccc |
| 661 | tctccggcaa caactgccgc ccaggagggg cagcccaaag tgcaccgagc ccggaaaacc |
| 721 | atgtccaaac ctagcaacgg acagcctcca atccctgaga agcggccccc tgaagtccag |
| 781 | catttccgca tgagtgatga catgcatctg gggaaggtga cttcagatgt ggccaaaagg |
| 841 | aggaagctga actctggtag cctgtccgag gacttgggct ctgccggggg ctcaggagat |
| 901 | ataatcctgg agaagggaga gcccaggccc ctggaggagt gggagacggt ggtgggcgat |
| 961 | gacttcagcc tgtactatga tgcgtactct gtggatgagc gggtggactc tgacagcaag |
| 1021 | tctgaagtcg aagctctagc tgaacagttg agtgaggagg aggaggagga agaggaggaa |
| 1081 | gaagaagaag aggaggagga ggaggaagag gaggaggagg aagaagagga cgaggagtcg |
| 1141 | ggcaatcagt cagacaggag cggttctagt ggccggcgca aggccaagaa gaaatggcgg |
| 1201 | aaagacagcc cgtgggtgaa gccatctaga aaacggcgga aacgagagcc tccgagggcc |
| 1261 | aaggagccaa gaggagtgaa tggtgtgggt tcctcagggc ccagtgagta catggaggtt |
| 1321 | cctctggggt ccctggagct gcccagcgag gggaccctct cccccaacca cgctggggtc |
| 1381 | tccaatgaca cgtcttcact ggagacagaa cgcgggtttg aggagctgcc cctctgcagc |
| 1441 | tgccgcatgg aggctcccaa gattgaccgc atcagcgaga gagcagggca caagtgcatg |
| 1501 | gccacagaga gtgtggatgg agagctcctg ggctgcaatg ctgccatcct taagcgggag |
| 1561 | accatgcggc cgtctagccg cgtggcgctg atggtgctct gtgaggccca tcgagcccgc |
| 1621 | atggtcaagc accattgctg cccgggctgc ggctacttct gcacagcggg caccttcctg |
| 1681 | gaatgccacc ccgactttcg tgtagctcac cgcttccata aggcctgcgt atcccagctc |
| 1741 | aatgggatgg tcttctgtcc ccactgtgga gaggatgcct cagaggccca ggaggtgacc |
| 1801 | attcctcggg gcgatggggg aacaccccca attggcaccg cagctcctgc tctgccaccc |
| 1861 | ctggcacatg atgccccagg gcgagcggat acctcccagc ctagcgcccg aatgcgaggg |
| 1921 | catggagagc cgcggcgccc gccctgtgat cccctggctg acaccatcga cagctcaggg |
| 1981 | ccttcactga ctctgcctaa tgggggctgc ctctccgctg tgggtctgcc cccagggccg |
| 2041 | ggcagggaag ccctggaaaa agccttggtc atccaggagt ctgagaggcg gaagaagctg |
| 2101 | cgattccacc cacggcagct gtacctgtcg gtgaagcagg gggagctgca gaaggtgatc |
| 2161 | cttatgctgt tagacaacct ggaccccaac ttccagagcg accagcagag caagcgcacg |
| 2221 | cccctgcacg cggccgccca gaaggggtcg gtagagatct gtcatgtgct gctgcaggca |
| 2281 | ggagccaaca tcaatgccgt agataagcaa caacgcacgc cactaatgga ggccgtggtg |
| 2341 | aacaaccacc tggaggtggc acgctacatg gtgcagttag gtggctgtgt ctacagcaag |
| 2401 | gaagaggatg gctccacctg tctacatcat gcagccaaaa ttgggaactt ggaaatggtc |
| 2461 | agcctgctac tgagcacagg acaggtggac gtcaatgccc aggacagtgg gggctggacg |
| 2521 | cccatcatct gggcagccga gcacaagcac atcgatgtga ttcgtatgct gctgacccgg |
| 2581 | ggtgccgatg tcaccctgac tgacaatgag gaaaacatct gcctgcactg ggcctccttc |
| 2641 | acgggtagtg ccgccatcgc tgaggtcctt ctgaatgccc agtgtgatct ccatgctgtc |
| 2701 | aactaccatg gggacacgcc cctgcacata gccgccaggg agagctacca tgactgtgtt |
| 2761 | ctgttgttcc tgtctcgtgg agccaaccct gagcttcgga acaaagaagg agacacggca |
| 2821 | tgggatctga ccccagagcg ctctgatgtg tggtttgcac tgcagctcaa tcgaaagctt |
| 2881 | aggcttgggg tagggaaccg ggctgtccgc accgagaaga tcatctgccg ggacgtagcc |
| 2941 | cgaggctatg agaatgtacc catcccctgt gtcaatggtg tggatgggga gccgtgcccg |
| 3001 | gaggactaca agtacatctc tgagaactgc gagacatcga ccatgaacat cgaccgcaac |
| 3061 | atcacccatc tgcagcactg cacgtgtgtg gatgactgct ccagctccaa ttgcctatgt |
| 3121 | ggtcagctca gtatccgatg ctggtatgac aaggacgggc ggctgctcca ggagtttaac |
| 3181 | aagatcgagc cccccctgat ctttgagtgt aaccaggcat gctcctgctg gagaagctgc |
| 3241 | aagaaccgcg tggtgcagag cggcatcaag gtacggctgc agctctaccg gactgccaag |
| 3301 | atgggctggg gggtccgagc cttgcagacc atcccccagg gcacgttcat ctgcgagtat |
| 3361 | gtaggagagc tgatctctga tgccgaggct gatgtgagag aggatgattc ttacctcttc |
| 3421 | gatttagata acaaggatgg cgaggtttac tgcattgatg cccgttacta tggcaacatc |
| 3481 | agccgattca ttaaccacct gtgtgacccc aacatcatcc ctgtccgggt tttcatgctg |
| 3541 | caccaagatc tacggttccc acgcattgcc ttcttcagct ccagggacat ccggactggg |
| 3601 | gaggagctgg gctttgacta cggtgaccga ttctgggaca tcaagagcaa gtatttcacc |
| 3661 | tgccagtgtg gctctgagaa gtgcaagcat tcagcggagg ccatcgccct ggagcagagc |
| 3721 | cgcctggccc ggctggaccc ccacccggag ctgctccctg acctcagctc cctgcccccc |
| 3781 | atcaacacct gaggactctt aaaatccagg ccgggcactg cccttcagac atttctccat |
| 3841 | cagagacccc agtaaggcct ggaaggtcga tggcccctct cccagagctg gtttctcact |
| 3901 | gggagtgcaa gtgacttcag ggctggcctt ccccactgag cctggcctca gttagctgat |
| 3961 | tgaagttggg cctctgccag ctgattttct gtgttctcaa taaatgttgg gtttggtaaa |
| 4021 | aaaaaa |
| SEQ ID NO: 4 | |
| 1 | mrglprgrgl mrargrgraa ptggrgrgrg gahrgrgrpr sllslpraqa swapqlpagl |
| 61 | tgppvpclps qgeapaemga lllekeprga aervhsslgd tpqseetlpk anpdslepag |
| 121 | psspasvtvt vgdegadtpv gaasligdep eslegdggri vlghatksfp sspskggacp |
| 181 | srakmsmtga gksppsvqs1 amrllsmpga qgaatagpep spattaaqeg qpkvhrarkt |
| 241 | mskpsngqpp ipekrppevq hfrmsddmhl gkvtsdvakr rklnsgslse dlgsaggsgd |
| 301 | iilekgeprp leewetvvgd dfslyydays vdervdsdsk sevealaeql seeeeeeeee |
| 361 | eeeeeeeeee eeeeeedees gnqsdrsgss grrkakkkwr kdspwvkpsr krrkreppra |
| 421 | keprgvngvg ssgpseymev plgslelpse gtlspnhagv sndtsslete rgfeelplcs |
| 481 | crmeapkidr iseraghkcm atesvdgell gcnaailkre tmrpssrval mvlceahrar |
| 541 | mvkhhccpgc gyfctagtfl echpdfrvah rfhkacvsql ngmvfcphcg edaseaqevt |
| 601 | iprgdggtpp igtaapalpp landapgrad tsqpsarmrg hgeprrppcd pladtidssg |
| 661 | psltlpnggc lsavglppgp grealekalv iqeserrkkl rfhprqlyls vkqgelqkvi |
| 721 | lmlldnldpn fqsdqqskrt plhaaaqkgs veichvllqa ganinavdkq qrtplmeavv |
| 781 | nnhlevarym vqlggcvysk eedgstclhh aakignlemv slllstgqvd vnaqdsggwt |
| 841 | piiwaaehkh idvirmlltr gadvtltdne eniclhwasf tgsaaiaevl lnaqcdlhav |
| 901 | nyhgdtplhi aaresyhdcv llflsrganp elrnkegdta wdltpersdv wfalq1nrkl |
| 961 | rlgvgnravr tekiicrdva rgyenvpipc vngvdgepcp edykyisenc etstmnidrn |
| 1021 | ithlqhctcv ddcsssnclc gqlsircwyd kdgrllqeth kiepplifec nqacscwrsc |
| 1081 | knrvvqsgik vrlqlyrtak mgwgvralqt ipqgtficey vgelisdaea dvreddsylf |
| 1141 | dldnkdgevy cidaryygni srfinhlcdp niipvrvfml hqdlrfpria ffssrdirtg |
| 1201 | eelgfdygdr fwdikskyft cqcgsekckh saeaialeqs rlarldphpe llpdlsslpp |
| 1261 | int |

In certain embodiments, human Jhdm2a corresponds to the nucleotide sequence set forth by NCBI reference No. NM_{—}018433.5, shown below as SEQ ID NO: 5, and the corresponding amino acid sequence set forth by NCBI reference No. NP_{—}060903.2, shown below as SEQ ID NO: 6.

| SEQ ID NO: 5 | |
|---|---|
| 1 | taatgggggt cgcccgggag tcggaagggg gaggggaaag ggaggaggca gccaaggaat |
| 61 | tgtttttttc tctggccccg ccctcgcccg gggggccaat ggtgatgatc tgtttccccc |
| 121 | ggagcctcgc ccagctcctg tgtttcagcc aatgagcggc ggaagcggct ccgagggggg |
| 181 | cgggtccggg aggctgtgcg tgtcttgtga gagctcttga accaagtcag cgctggagtc |
| 241 | ggctaggcgg ctggaaacgg cggctgccgc cggtgactca gggaggcggg aggcggggga |
| 301 | ggagctcttc ctgcaggcgt ggaaaccatg gtgctcacgc tcggagaaag ttggccggta |
| 361 | ttggtgggga ggaggtttct cagtctgtcc gcagccgacg gcagcgatgg cagccacgac |
| 421 | agctgggacg tggagcgcgt cgccgagtgg ccctggctct ccgggaccat tcgagctgtt |
| 481 | tcccacaccg acgttaccaa gaaggatctg aaggtgtgtg tggaatttga tggggaatct |
| 541 | tggaggaaaa gaagatggat agaagtctac agccttctaa ggagagcatt tttagtagaa |
| 601 | cataatttgg ttttagctga acgaaagtca cctgaaattt ctgaacgaat tgtacagtgg |
| 661 | cctgcaataa cgtacaaacc tctgttggac aaagctggtt tgggatccat aacttctgtt |
| 721 | cgctttctgg gagatcaaca aagagtattt ctttctaaag accttttgaa gcctatacag |
| 781 | gatgtaaaca gtcttcgact ttctcttacg gataatcaga ttgtcagtaa agaatttcaa |
| 841 | gctttgattg tgaagcattt agatgaaagc catcttttaa aaggtgacaa aaacttagtt |
| 901 | ggttcagaag taaaaattta tagcttggac ccatctactc agtggttttc agcaaccgtt |
| 961 | ataaatggaa acccagcatc aaaaactctt caagtcaact gtgaggagat tccagcactg |
| 1021 | aaaattgttg atccgtcact gattcatgtt gaagttgtac acgataacct tgtgacatgt |
| 1081 | ggtaattctg caagaattgg agctgtaaaa cgcaagtctt ctgagaataa tggaaccctg |
| 1141 | gtttccaaac aagcaaaatc ttgctctgag gcctctccca gtatgtgtcc tgtgcagtct |
| 1201 | gtacctacaa cagtttttaa ggagatactg cttggctgta ctgcggcaac tccacctagt |
| 1261 | aaggacccaa gacagcaaag tactccccag gctgccaact ctccacctaa ccttggagca |
| 1321 | aaaattcctc aaggatgtca taaacaaagt ttaccagagg aaatttcttc ctgtctaaat |
| 1381 | acaaagtctg aagctctgag aacaaaacca gatgtctgca aagcagggtt gctctcaaag |
| 1441 | tcctctcaga ttggaactgg agacttgaaa attctgactg agccaaaagg cagctgtact |
| 1501 | cagcctaaga caaacactga tcaggaaaac agattggagt ctgttccaca agcattgact |
| 1561 | ggccttccta aggagtgctt acctacaaag gcttcttcta aggcagaatt ggaaattgcc |
| 1621 | aatcctcctg aactgcagaa gcacctagaa catgcacctt ccccatcgga tgtttcaaat |
| 1681 | gcaccagaag tgaaagcagg tgtcaatagt gatagcccta ataactgttc aggaaaaaag |
| 1741 | gtagaacctt cagctttagc ttgccgatca cagaatttaa aggaatcttc agtaaaagta |
| 1801 | gataatgaaa gctgttgttc aagaagcaac aataaaatcc agaatgcccc atccaggaag |
| 1861 | tcggttttga cagacccagc taaactcaaa aagctgcaac agagtggcga ggccttcgta |
| 1921 | caggatgatt cttgtgtgaa catcgtggca cagttgccta aatgccgaga gtgtcgcttg |
| 1981 | gacagtctcc gcaaggataa ggagcaacag aaggactcac ctgtgttttg ccgcttcttt |
| 2041 | cacttcagga ggttacaatt caacaaacat ggtgtgttgc gggtagaagg cttcttaaca |
| 2101 | ccaaacaagt atgacaatga agcaattggc ttgtggttac ctttaaccaa aaacgttgtg |
| 2161 | gggattgatt tggacacagc aaagtacatc ttggccaaca ttggagacca cttctgtcaa |
| 2221 | atggtgattt ctgaaaagga agctatgtca actattgagc cacacagaca ggttgcttgg |
| 2281 | aagcgagctg tcaaaggtgt tcgagaaatg tgtgatgtgt gcgacaccac catcttcaac |
| 2341 | ctgcactggg tgtgtcctcg gtgtgggttt ggagtatgtg tggactgcta ccggatgaag |
| 2401 | agaaagaatt gccaacaggg tgctgcttac aagactttct cttggctaaa atgtgtgaag |
| 2461 | agtcagatac atgaaccaga gaacttaatg cccacacaga tcattcctgg aaaagcactc |
| 2521 | tatgatgttg gagacattgt tcattctgta agagcgaaat ggggaataaa ggcaaactgc |
| 2581 | ccttgttcaa acaggcaatt caaactcttt tcaaagccag cctcaaagga agacctaaaa |
| 2641 | cagacttctt tagctggaga aaaaccgact cttggtgcag tgctccagca gaatccctca |
| 2701 | gtgttggagc cagcagctgt gggtggggaa gcagcctcca agccagccgg cagcatgaag |
| 2761 | cctgcctgtc cagccagcac atctcctcta aactggctgg ccgacctaac cagcgggaat |
| 2821 | gtcaacaagg aaaacaagga aaaacaacca acaatgccaa ttttaaagaa tgaaatcaaa |
| 2881 | tgccttccac ccctcccacc tttaagcaaa tccagcacag tcctccatac gtttaacagc |
| 2941 | acaattttga cacccgtaag caacaacaat tctggtttcc tccggaatct cttgaattct |
| 3001 | tctacaggaa agacagaaaa tggactcaag aatacaccaa aaatccttga tgacatcttt |
| 3061 | gcctctttgg tgcaaaataa gacgacttct gatttatcta agaggcctca aggactaacc |
| 3121 | atcaagccca gcattctggg ctttgacact cctcactatt ggctttgtga taatcgcttg |
| 3181 | ctgtgcttgc aagaccccaa caataagagc aactggaatg tgtttaggga gtgctggaaa |
| 3241 | caagggcagc cagtgatggt gtctggagtg catcataaat tgaactctga actttggaaa |
| 3301 | cctgaatcct tcaggaaaga gtttggtgag caggaagtag acctagttaa ttgtaggacc |
| 3361 | aatgaaatca tcacaggagc cacagtagga gacttctggg atggatttga agatgttcca |
| 3421 | aatcgtttga aaaatgaaaa agaaccaatg gtgttgaaac ttaaggactg gccaccagga |
| 3481 | gaagatttta gagatatgat gccttccagg tttgatgatc tgatggccaa cattccactg |
| 3541 | cccgagtaca caaggcgaga tggcaaactg aatttggcct ctaggctgcc aaactacttt |
| 3601 | gttcggccag atctgggccc caagatgtat aatgcttatg gattaatcac tcctgaagat |
| 3661 | cggaaatatg gaacaacaaa tcttcactta gatgtatctg atgcagctaa tgtcatggtc |
| 3721 | tatgtgggaa ttcccaaagg acagtgtgag caagaagaag aagtccttaa gaccatccaa |
| 3781 | gatggagatt ctgacgaact cacaataaag cgatttattg aaggaaaaga gaagccagga |
| 3841 | gcactgtggc acatatatgc tgcaaaggac acggagaaga taagggaatt tcttaaaaag |
| 3901 | gtatcagaag agcaaggtca agaaaaccca gcagaccacg atcctattca tgatcaaagc |
| 3961 | tggtatttag accgatcatt aagaaaacgt cttcatcaag agtatggagt tcaaggctgg |
| 4021 | gctattgtac agtttcttgg ggatgtggtg tttatccegg caggagctcc acatcaggtt |
| 4081 | cataacttat atagctgcat caaagtggct gaagattttg tttctccaga gcatgttaaa |
| 4141 | cactgcttct ggcttactca ggaattccga tatctgtcac agactcatac caatcacgaa |
| 4201 | gataaattac aggtgaagaa tgttatctac catgcagtga aagatgcagt tgctatgctg |
| 4261 | aaagccagtg aatccagttt tggcaaacct taatctccct gcacattgga aatgaattac |
| 4321 | aggcagctgt tcaaactctt caggcaggat tcctgtggac tttgagattc atgttacctc |
| 4381 | atcttctttt ttaaactgta cccaacttgt gagggtactc tgtctaatgt atatttctag |
| 4441 | tgtttacaga cagtaaatgt gtatatgtag taactattta cagaacatgc atccttaaac |
| 4501 | tgtgacttct cacctagtgc agaactttta ccaggctgta aaagcaaaac ctcgtatcag |
| 4561 | ctctggaaca atacctgcag ttattcttca gctgtttgga caacttagat tgggtttata |
| 4621 | actattagga atcactgcac agtttatttg ggttgtgttt tgtgtctgag tcccctccct |
| 4681 | catcccttag ggtccagaag agcaatggag gaagtgacag ctaatgttgc agttcttatt |
| 4741 | gtatggcata ggactggcat tatatagcag aaatcaacta ctgtacaatt tcttggggtt |
| 4801 | aaccatcttt agttaaatgg aattttaatt taaatgacgc tttgctaatt ttaagtgtta |
| 4861 | agcattttgc attaaaatat tcatataata aaaaaaaaaa aaaaaaa |
| SEQ ID NO: 6 | |
| 1 | mvltlgeswp vlvgrrflsl saadgsdgsh dswdvervae wpwlsgtira vshtdvtkkd |
| 61 | lkvcvefdge swrkrrwiev ysllrraflv ehnlvlaerk speiserivq wpaitykpll |
| 121 | dkaglgsits vrflgdqqry flskdllkpi qdvnslrlsl tdnqivskef qalivkhlde |
| 181 | shllkgdknl vgsevkiysl dpstqwfsat vingnpaskt lqvnceeipa lkivdpslih |
| 241 | vevvhdnlvt cgnsarigav krkssenngt lvskqakscs easpsmcpvq svpttvfkei |
| 301 | llgctaatpp skdprqqstp qaansppnlg akipqgchkq slpeeisscl ntksealrtk |
| 361 | pdvckaglls kssqigtgdl kiltepkgsc tqpktntdqe nrlesvpqal tglpkeclpt |
| 421 | kasskaelei anppelqkhl ehapspsdvs napevkagvn sdspnncsgk kvepsalacr |
| 481 | sqnlkessvk vdnesccsrs nnkiqnapsr ksvltdpakl kklqqsgeaf vqddscvniv |
| 541 | aqlpkcrecr ldslrkdkeq qkdspvfcrf fhfrrlqfnk hgvlrvegfl tpnkydneai |
| 601 | glwlpltknv vgidldtaky ilanigdhfc qmvisekeam stiephrqva wkravkgvre |
| 661 | mcdvcdttif nlhwvcprcg fgvcvdcyrm krkncqqgaa yktfswlkcv ksqihepenl |
| 721 | mptqiipgka lydvgdivhs vrakwgikan cpcsnrqfkl fskpaskedl kqtslagekp |
| 781 | tlgavlqqnp svlepaavgg eaaskpagsm kpacpastsp lnwladltsg nvnkenkekq |
| 841 | ptmpilknei kclpplppls ksstvlhtfn stiltpvsnn nsgflrnlln sstgktengl |
| 901 | kntpkilddi faslvqnktt sdlskrpqgl tikpsilgfd tphywlcdnr llclqdpnnk |
| 961 | snwnvfrecw kqgqpvmvsg vhhklnselw kpesfrkefg eqevdlyncr tneiitgatv |
| 1021 | gdfwdgfedv pnrlknekep mvlklkdwpp gedfrdmmps rfddlmanip 1peytrrdgk |
| 1081 | lnlasrlpny fvrpdlgpkm ynayglitpe drkygttnlh ldvsdaanvm vyvgipkgqc |
| 1141 | eqeeevlkti qdgdsdelti krfiegkekp galwhiyaak dtekireflk kvseeqgqen |
| 1201 | padhdpihdq swyldrslrk rlhqeygvqg waivqflgdv vfipagaphq vhnlyscikv |
| 1261 | aedfvspehv khcfwltqef rylsqthtnh edklqvknvi yhavkdavam lkasessfgk |
| 1321 | p |

In certain embodiments, mouse Jhdm2a corresponds to the nucleotide sequence set forth by NCBI reference No. NM_{—}173001, shown below as SEQ ID NO: 7, and the corresponding amino acid sequence set forth by NCBI reference No. NP_{—}766589.1, shown below as SEQ ID NO: 8.

| SEQ ID NO: 7 | |
|---|---|
| 1 | aagtgtcgag tcgcgagcga gtccacggcg gctccgaggc cgctcggggc ggggatcggt |
| 61 | cgctgagacg ggccctaggc actaagaggg agccttttct ttaacagggc gaggggacga |
| 121 | acacttaggc aaaagcactg gcgccgcggc tcagtcctcc cttctctctc tcagtgtcca |
| 181 | gctttgaaag ggaggagccc ttcctgctgg cgtggaaacc atggtgctca cgctcggaga |
| 241 | aagttggcca gtattggtgg ggaagcgatt cctcagtctg tccgcagccg aaggcaacga |
| 301 | aggcggccag gacaactggg acttggagcg cgttgccgag tggccctggc tgtcggggac |
| 361 | cattcgagct gtttcccaca ccgacgttac taagaaagac ttgaaggtgt gtgtggagtt |
| 421 | tgatggggag tcttggagaa agagaagatg gatagatgtc tacagccttc agagaaaagc |
| 481 | atttttagta gagcataacc tggttttggc agaacgaaaa tcacctgaag ttcctgagca |
| 541 | agttattcag tggcctgcaa taatgtacaa atctcttcta gacaaagctg gcttgggagc |
| 601 | cataacttct gttcggtttc ttggagatca acaaagtgta tttgtttcca aagacctttt |
| 661 | gaaacctata caggatgtta acagtcttcg gctttccctt actgataatc agacagtcag |
| 721 | taaggaattt caagctttga ttgtaaaaca tttggatgaa agccatcttt tacaaggtga |
| 781 | caagaacctt gttggttcag aagtaaaaat ttatagcttg gacccatcta ctcagtggtt |
| 841 | ttcagcaact gttgtacatg gaaacccatc atccaaaact cttcaagtca actgtgagga |
| 901 | gattccagca ctgaaaattg tcgacccagc actgattcat gttgaagttg tacatgacaa |
| 961 | ctttgtgaca tgtggtaatt ctacaagaac tggagctgta aaacgcaagt cttctgagaa |
| 1021 | taacggaagt tcggtttcta aacaagcaaa atcttgttct gaggcctctc ccagtatgtg |
| 1081 | tcctgtacag tctgttccca caacagtgtt taaggagatc ctgcttggct gtactgcagc |
| 1141 | aactccatct agcaaggacc caagacagca aaatactccc caggcagcca attctccacc |
| 1201 | taacattgga gcaaaacttc ctcaaggatg tcataagcag aacttaccag aagaactttc |
| 1261 | ttcctgtcta aacacaaaac ctgaagtacc gagaacaaaa ccagatgtct gcaaagaagg |
| 1321 | attactttct tcaaaatctt ctcaggttgg agctggagac ttgaaaattc tgagtgagcc |
| 1381 | caaaggtagc tgtatccagc ctaaaacaaa cactgatcag gagagcagac tggagtctgc |
| 1441 | tccacagcca gtcactggcc ttccaaagga gtgcttgcct gcaaagactt cctctaaggc |
| 1501 | agaactggac attgccacca ctcctgaact gcagaagcat ctagaacatg cagcttccac |
| 1561 | atccgatgac ctttcagata agccagaagt gaaagcaggt gtcactagcc ttaatagttg |
| 1621 | tgcagaaaag aaggtcgaac cttcacattt aggttcccag tcacagaatt taaaggaaac |
| 1681 | ttcagtaaaa gtagataatg aaagctgttg tacaagaagc agtaataaaa cccagactcc |
| 1741 | cccagcccgg aagtcagttt tgacagaccc agataaagtc aggaagctgc agcagagcgg |
| 1801 | agaggccttt gttcaggatg actcctgtgt taacatcgtg gcacagctgc ccaagtgtcg |
| 1861 | ggagtgtcga ctagacagcc tgcgcaagga taaggaccag cagaaggact ctcctgtgtt |
| 1921 | ttgtcgcttt ttccacttca ggagattaca attcaacaag catggtgtgt tgcgggtaga |
| 1981 | aggcttctta acaccaaaca agtatgacag tgaagcgatt ggcttgtggc tgcctttgac |
| 2041 | caaaaatgtt gtggggactg atttggacac agcaaaatat atcctggcca atattggaga |
| 2101 | ccacttctgt caaatggtga tttctgagaa ggaagctatg tcgactattg agccacacag |
| 2161 | acaggttgct tggaaacgag ctgtcaaagg agttagagaa atgtgtgatg tgtgtgacac |
| 2221 | aaccattttc aacctgcact gggtgtgccc tcggtgtggg tttggagtat gtgtagattg |
| 2281 | ctaccggatg aagaggaaga actgccaaca gggtgctgcc tacaagactt tctcttggat |
| 2341 | aaggtgtgtg aagagtcaga tacatgagcc tgagaacctg atgcccacac agattattcc |
| 2401 | tggcaaagcc ctctacgatg ttggagacat tgtgcattct gtcagagcaa aatggggcat |
| 2461 | aaaggccaat tgtccctgct ccaacaggca gttcaagctc ttctcaaagc cagccttaaa |
| 2521 | ggaagacctg aaacagacat ccttgtctgg agaaaaacca actcttggga ccatggtcca |
| 2581 | gcaaagttcc cctgttttgg agccagtggc tgtgtgcggg gaagcagcct ccaagccagc |
| 2641 | cagcagcgtg aagcccacct gtcccaccag cacttcacct ttaaactggc tagctgacct |
| 2701 | taccagtggg aatgtcaaca aggagaataa ggaaaaacag ctgactatgc caattttaaa |
| 2761 | gaatgaaatc aaatgccttc cacccttgcc ccctctgaac aagcccagca cagtcctcca |
| 2821 | tacttttaac agcaccatct tgacacctgt gagcaacaat aattcaggtt tccttagaaa |
| 2881 | tcttttgaat tcatccacag caaagacaga aaatggattg aaaaacacac ccaaaattct |
| 2941 | tgatgacatc tttgcctctt tggtgcaaaa caagacttct tctgattcat ccaagaggcc |
| 3001 | tcaaggactg acaatcaagc ctagcattct tggctttgac actcctcact actggctgtg |
| 3061 | tgacaaccgc ctgctgtgct tgcaagaccc caacaataag agcaattgga atgtttttag |
| 3121 | ggaatgctgg aaacaagggc agccagtgat ggtgtcgggc gtgcatcata aattaaacac |
| 3181 | tgaactctgg aaacccgagt ccttcagaaa agagtttggt gagcaggaag tagacctagt |
| 3241 | caattgtagg accaatgaaa tcatcactgg agccacagtc ggagacttct gggatggatt |
| 3301 | tgaagatgtt ccaaaccgtt tgaaaaacga caaagaaaaa gaaccaatgg tgttgaaact |
| 3361 | taaggactgg ccgccaggag aagacttcag agacatgatg ccttccaggt ttgatgatct |
| 3421 | gatggccaac attccactgc ctgagtacac caggcgagat ggcaaactga acctggcttc |
| 3481 | cagactgcca aactactttg tacggccaga cctgggcccc aagatgtaca atgcttatgg |
| 3541 | attgatcact ccagaggatc ggaaatatgg gaccacaaat cttcacttag atgtatctga |
| 3601 | tgcagccaat gtcatggttt atgtgggaat tcccaaagga cagtgtgaac aagaagaaga |
| 3661 | agtccttaga accatccaag atggagattc tgatgaactc acaatcaaga gatttattga |
| 3721 | aggaaaagag aagccaggag ccctttggca catatatgct gctaaagaca cagagaagat |
| 3781 | aagagaattc cttaaaaagg tatcagagga gcagggtcaa gacaaccctg cagaccatga |
| 3841 | ccctatccac gatcagagct ggtatttaga ccgatcgctg agaaagcgcc tctatcaaga |
| 3901 | gtacggcgtg caaggctggg ctattgtaca gtttcttggg gatgtggtgt ttatcccagc |
| 3961 | aggagcgcca catcaggttc ataacttata cagctgtatc aaagtggctg aagactttgt |
| 4021 | gtctccagag catgttaaac actgcttctg gcttactcag gaattccgtt acttgtcaca |
| 4081 | gactcatacc aaccatgaag ataaattgca ggtgaaaaat gttatctacc atgcagtgaa |
| 4141 | agatgcagtt gctatgctga aagccagtga atccagtttg ggcaaacctt aactcttctc |
| 4201 | tgcacaatgg agatgaatta ttggcagctg atcaaactct tcaggcagga ttcctgtgga |
| 4261 | ctttgagatt tcctgttacc tcatcttctt ttttaaagta cacctgactt gggagggtac |
| 4321 | tgtctctaat gtatatttct agtgtttaca gacactaagt gtgtatatgt agtaactatt |
| 4381 | tacagaccac gcatccttat actgtgactt cacctagatc ttctaccaag ctgaagaccc |
| 4441 | tgctggctct gaaacaatcc ttgcagttac tccccagctg ttcgtctgga cagctcattc |
| 4501 | aagtggattt ttaactatta gggatcactg cgaagtttcg ttggatttta ttttatgtcc |
| 4561 | ttcagagcac cctcccccac ccactagggt ccagaagagc aatggaggaa gtgacagcta |
| 4621 | atggtgcagt tctaaatata tattgcatag gactggcatt atatagcaga aataactact |
| 4681 | gtataattct tggggttaac catctttagt taatggaatt ttaatttaaa tgaagctttg |
| 4741 | ctaattttaa gtggtaagca ttttgcatta aaatattcct ataatatttt gtcgctgttc |
| 4801 | tttgtccttt attctttgtt tactctctcg aaaataaaag ggctaaacta ttgaaaaaaa |
| 4861 | aaaaaaaa |
| SEQ ID NO: 8 | |
| 1 | mvltlgeswp vlvgkrflsl saaegneggq dnwdlervae wpwlsgtira vshtdvtkkd |
| 61 | lkvcvefdge swrkrrwidv yslqrkaflv ehnlvlaerk spevpeqviq wpaimyksll |
| 121 | dkaglgaits vrflgdqqsv fvskdllkpi qdvnslrlsl tdnqtvskef qalivkhlde |
| 181 | shllqgdknl vgsevkiysl dpstqwfsat vvhgnpsskt lqvnceeipa lkivdpalih |
| 241 | vevvhdnfvt cgnstrtgav krkssenngs syskqakscs easpsmcpvq svpttvfkei |
| 301 | llgctaatps skdprqqntp qaansppnig aklpqgchkq nlpeelsscl ntkpevprtk |
| 361 | pdvckeglls skssqvgagd lkilsepkgs ciqpktntdq esrlesapqp vtglpkeclp |
| 421 | aktsskaeld iattpelqkh lehaastsdd lsdkpevkag vtslnscaek kvepshlgsq |
| 481 | sqnlketsvk vdnescctrs snktqtppar ksvltdpdkv rklqqsgeaf vqddscvniv |
| 541 | aqlpkcrecr ldslrkdkdq qkdspvfcrf fhfrrlqfnk hgvlrvegfl tpnkydseai |
| 601 | glwlpltknv vgtdldtaky ilanigdhfc qmvisekeam stiephrqva wkravkgvre |
| 661 | mcdvcdttif nlhwvcprcg fgvcvdcyrm krkncqqgaa yktfswircv ksqihepenl |
| 721 | mptqiipgka lydvgdivhs vrakwgikan cpcsnrqfkl fskpalkedl kqtslsgekp |
| 781 | tlgtmvqqss pvlepvavcg eaaskpassv kptcptstsp lnwladltsg nvnkenkekq |
| 841 | ltmpilknei kclpplppin kpstvlhtfn stiltpvsnn nsgflrnlln sstaktengl |
| 901 | kntpkilddi faslvqnkts sdsskrpqgl tikpsilgfd tphywlcdnr llclqdpnnk |
| 961 | snwnvfrecw kqgqpvmvsg vhhklntelw kpesfrkefg eqevdlyncr tneiitgatv |
| 1021 | gdfwdgfedv pnrlkndkek epmvlklkdw ppgedfrdmm psrfddlman iplpeytrrd |
| 1081 | gklnlasrlp nyfvrpdlgp kmynayglit pedrkygttn lhldvsdaan vmvyvgipkg |
| 1141 | qceqeeevlr tiqdgdsdel tikrfiegke kpgalwhiya akdtekiref lkkvseeqgq |
| 1201 | dnpadhdpih dqswyldrsl rkrlyqeygv qgwaivqflg dvvfipagap hqvhnlysci |
| 1261 | kvaedfvspe hvkhcfwltq efrylsqtht nhedklqvkn viyhavkdav amlkasessl |
| 1321 | gkp |

### Methods for Reprogramming Somatic Cells

The present invention provides methods for reprogramming somatic cells. Preferably, the somatic cells are reprogrammed to a less differentiated, or de-differentiated, state. De-differentiation refers to a process whereby a cell changes from a more specialized function to a cell that has a less specialized function. Through the process of de-differentiation a cell can become pluripotent. A pluripotent cell is able to differentiate into many cell types.

Accordingly, the invention features a method for reprogramming one or more somatic cells comprising treating the cells with one or more agents that induces de-differentiation, wherein the agent is selected from a histone methyltransferase inhibitor or a histone demethylase activator, thereby generating a reprogrammed cell.

In preferred examples, the cells have a marker.

In certain embodiments, the marker is a marker gene. A marker gene is any gene that enables cell sorting and selection by introducing the marker gene into cells. Specifically, a drug resistance gene, a fluorescent protein gene, a luminescent enzyme gene, a chromogenic enzyme gene or a gene comprising a combination of any of these.

Included as exemplary fluorescent protein gene are the GFP (green fluorescent protein) gene, the YFP (yellow fluorescent protein) gene, the RFP (red fluorescent protein) gene, the aequorin gene. Cells expressing these fluorescent protein genes can be detected with a fluorescence microscope. The cells can also be selected by separation and selection using a cell sorter and the like on the basis of differences in fluorescence intensity.

Included as an exemplary as the drug resistance gene are the neomycin resistance gene (neo), tetracycline resistance gene (tet), kanamycin resistance gene, zeocin resistance gene (zeo), hygromycin resistance gene (hygro), puromycin resistance gene (pur). When cells are cultured using a medium comprising each drug (referred to as a selection medium), only those cells incorporating and expressing the drug resistance gene survive. Therefore, by culturing cells using a selection medium, it is possible to easily select cells comprising a drug resistance gene.

All the above-described marker genes are well known to those skilled in the art; vectors harboring such a marker gene are commercially available from Invitrogen, Inc., Amersham Biosciences, Inc., Promega, Inc., MBL (Medical & Biological Laboratories Co., Ltd.) and the like.

Accordingly, the invention features a method for reprogramming one or more somatic cells comprising treating the cells with one or more agents that induces de-differentiation; and detecting the expression of one or more markers, where at least one marker indicates cell reprogramming; selecting a cell that expresses the one or more markers; thereby generating a reprogrammed cell.

In preferred embodiments, the invention makes use of the Cre-lox recombinase system. The Cre-lox system has been successfully applied in mammalian cell cultures, yeasts, plants, mice, and other organisms. The Cre-lox system is a viral recombination system that requires only two components-(1) Cre recombinase: an enzyme that catalyzes recombination between two LoxP sites and (2) LoxP sites: specific 34-base pair (bp) sequences consisting of an 8-bp core sequence, where recombination takes place, and two flanking 13-bp inverted repeats. The outcome of a Cre-lox recombination is determined by the orientation and location of flanking loxP sites. (A) If the loxP sites are oriented in opposite directions, Cre recombinase mediates the inversion of the foxed segment. (B) If the loxP sites are located on different chromosomes (trans arrangement), Cre recombinase mediates a chromosomal translocation. (C) If the loxP sites are oriented in the same direction on a chromosome segment (cis arrangement), Cre recombinase mediates a deletion of the foxed segment. In certain cases, a Cre transgene under the control of an inducible promoter can be introduced so the target DNA can be deleted inside selected cells of a transgenic organism at a desired time. Accordingly, the somatic cell may in certain examples comprise a Cre recombinase protein, and the embryonic cell comprise a fluorescent Cre recombination excision reporter, and so detection of the fluorescent Cre recombination reporter is used to monitor cell fusion. The somatic cell can be further engineered to stably co-express Cre and the puromycin resistance gene. The embryonic cell comprises CAG-loxP-LacZ::neomycin-polyA-loxP-DsRed.T3 as the fluorescent Cre recombination excision reporter.

The somatic cell may further comprise GFP, and detection of GFP is then used to identify an agent that alters somatic cell reprogramming.

In preferred embodiments, the cells are contacted in the presence of polyethyleneglycol (PEG).

The treatment with one or more agents may be contacting the cells with an agent, or may be transfecting the cells with one or more pluripotency genes, or may be both. If the treatments are both, they may be concurrent, or may be sequential, in any order.

Methods for preparing reprogramming cells according to the methods of the present invention are not particularly limited. Any method may be employed as long as the reprogramming factor, e.g. the agents that induce de-differentiation can contact the somatic cells under an environment in which the somatic cells and the induced pluripotent stem cells can proliferate. One such advantage of the present invention is that an induced pluripotent stem cell can be prepared by contacting a nuclear reprogramming factor with a somatic cell in the absence of eggs, embryos, or embryonic stem (ES) cells.

In preferred embodiments, the somatic cells may be primary cells or immortalized cells. For example, the cells may be primary cells (non-immortalized cells), such as those freshly isolated from an animal, or may be derived from a cell line (immortalized cells). In other embodiments, the somatic cells in the present invention are mammalian cells, such as, for example, human cells or mouse cells. They may be obtained by well-known methods, from different organs, e.g., skin, lung, pancreas, liver, stomach, intestine, heart, reproductive organs, bladder, kidney, urethra and other urinary organs, etc., generally from any organ or tissue containing live somatic cells. Mammalian somatic cells useful in the present invention include, for example, adult stem cells, sertoli cells, endothelial cells, granulosa epithelial, neurons, pancreatic islet cells, epidermal cells, epithelial cells, hepatocytes, hair follicle cells, keratinocytes, hematopoietic cells, melanocytes, chondrocytes, lymphocytes (B and T lymphocytes), erythrocytes, macrophages, monocytes, mononuclear cells, fibroblasts, cardiac muscle cells, and other muscle cells, etc. generally any live somatic cells. “Somatic cells”, as used herein, also includes adult stem cells. An adult stem cell is a cell that is capable of giving rise to all cell types of a particular tissue. Exemplary adult stem cells include neural stem cells, hematopoietic stem cells, and mesenchymal stem cells.

In another embodiment of the invention, the engineered somatic cells are obtained from a transgenic mouse comprising such engineered somatic cells. Such transgenic mouse can be produced using standard techniques known in the art. For example, Bronson et al. describe a technique for inserting a single copy of a transgene into a chosen chromosomal site. See Bronson et al., 1996. Briefly, a vector containing the desired integration construct containing a pluripotency gene is introduced into ES cells by standard techniques known in the art. The resulting ES cells are screened for the desired integration event, in which the knock-in vector is integrated into the desired endogenous pluripotency gene locus such that, for example a selectable marker is integrated into the genomic locus of the pluripotency gene and is under the control of the pluripotency gene promoter. The desired ES cell is then used to produce transgenic mouse in which all cell types contain the correct integration event. Desired types of cells may be selectively obtained from the transgenic mouse and maintained in vitro.

Alternatively, engineered somatic cells of the present invention may be produced by direct introduction of the desired construct into somatic cells. DNA construct may be introduced into cells by any standard technique known in the art, such as viral transfection (e.g. using an adenoviral system) or liposome-mediated transfection.

For example, a gene product as described herein may be added to a medium. Alternatively, by using a vector containing a gene that is capable of expressing the reprogramming factor of the present invention, a means of transducing said gene into a somatic cell may be employed. When such vector is used, two or more kinds of genes may be incorporated into the vector, and each of the gene products may be simultaneously expressed in a somatic cell.

A viral-based gene transfer and expression vector enables efficient and robust delivery of genetic material to most cell types, including non-dividing and hard-to-transfect cells (primary, blood, stem cells) in vitro or in vivo. Viral-based constructs integrated into genomic DNA result in high expression levels. In addition to a DNA segment that encodes a gene of interest, the vectors may include a transcription promoter and a polyadenylation signal operatively linked, upstream and downstream, respectively, to the DNA segment. The vector can include a single DNA segment encoding a single potency-determining factor or a plurality of potency-determining factor-encoding DNA segments. A plurality of vectors can be introduced into a single somatic cell. The vector can optionally encode a selectable marker to identify cells that have taken up and express the vector. As an example, when the vector confers antibiotic resistance on the cells, antibiotic can be added to the culture medium to identify successful introduction of the vector into the cells. Integrating vectors can be employed, as in the examples, to demonstrate proof of concept. Retroviral (e.g., lentiviral) vectors are integrating vectors; however, non-integrating vectors can also be used. Such vectors can be lost from cells by dilution after reprogramming, as desired. A suitable non-integrating vector is an Epstein-Barr virus (EBV) vector. Ren C, et al., Acta. Biochim. Biophys. Sin. 37:68-73 (2005); and Ren C, et al., Stem Cells 24:1338-1347 (2006), each of which is incorporated herein by reference as if set forth in its entirety.

The vectors described herein can be constructed and engineered using art-recognized techniques to increase their safety for use in therapy and to include suitable expression elements and therapeutic genes. Standard techniques for the construction of expression vectors suitable for use in the present invention are well-known to one of ordinary skill in the art and can be found in such publications such as Sambrook J, et al., “Molecular cloning: a laboratory manual,” (3rd ed. Cold Spring Harbor Press, Cold Spring Harbor, N.Y. 2001), incorporated herein by reference as if set forth in its entirety.

During development of multicellular organisms, different cells and tissues acquire different programs of gene expression. These distinct gene expression patterns appear to be regulated to a considerable degree by epigenetic modifications such as DNA methylation, histone modifications and various chromatin-binding proteins. Thus each cell type within a multicellular organism is thought to have a unique epigenetic signature which is thought to become fixed once cells differentiate or exit the cell cycle. In addition, some cells undergo epigenetic reprogramming during normal development or certain disease situations. Accordingly, treatment with agents that alter DNA methylation, histone modifications and various chromatin-binding proteins are contemplated by the present invention.

In particular examples, agents that target histone demethylase family of enzymes, histone methyltransferase family of enzymes are preferred.

An additional preferred target of the invention is the transcription factor Nanog. In certain embodiments, human Nanog corresponds to the nucleotide sequence set forth by NCBI reference No. NM_{—}024865, shown below as SEQ ID NO: 9, and the corresponding amino acid sequence set forth by NCBI reference No. NP_{—}079141, shown below as SEQ ID NO: 10.

| SEQ ID NO: 9 | |
|---|---|
| 1 | attataaatc tagagactcc aggattttaa cgttctgctg gactgagctg gttgcctcat |
| 61 | gttattatgc aggcaactca ctttatccca atttcttgat acttttcctt ctggaggtcc |
| 121 | tatttctcta acatcttcca gaaaagtctt aaagctgcct taaccttttt tccagtccac |
| 181 | ctcttaaatt ttttcctcct cttcctctat actaacatga gtgtggatcc agcttgtccc |
| 241 | caaagcttgc cttgctttga agcatccgac tgtaaagaat cttcacctat gcctgtgatt |
| 301 | tgtgggcctg aagaaaacta tccatccttg caaatgtctt ctgctgagat gcctcacacg |
| 361 | gagactgtct ctcctcttcc ttcctccatg gatctgctta ttcaggacag ccctgattct |
| 421 | tccaccagtc ccaaaggcaa acaacccact tctgcagaga agagtgtcgc aaaaaaggaa |
| 481 | gacaaggtcc cggtcaagaa acagaagacc agaactgtgt tctcttccac ccagctgtgt |
| 541 | gtactcaatg atagatttca gagacagaaa tacctcagcc tccagcagat gcaagaactc |
| 601 | tccaacatcc tgaacctcag ctacaaacag gtgaagacct ggttccagaa ccagagaatg |
| 661 | aaatctaaga ggtggcagaa aaacaactgg ccgaagaata gcaatggtgt gacgcagaag |
| 721 | gcctcagcac ctacctaccc cagcctttac tcttcctacc accagggatg cctggtgaac |
| 781 | ccgactggga accttccaat gtggagcaac cagacctgga acaattcaac ctggagcaac |
| 841 | cagacccaga acatccagtc ctggagcaac cactcctgga acactcagac ctggtgcacc |
| 901 | caatcctgga acaatcaggc ctggaacagt cccttctata actgtggaga ggaatctctg |
| 961 | cagtcctgca tgcagttcca gccaaattct cctgccagtg acttggaggc tgccttggaa |
| 1021 | gctgctgggg aaggccttaa tgtaatacag cagaccacta ggtattttag tactccacaa |
| 1081 | accatggatt tattcctaaa ctactccatg aacatgcaac ctgaagacgt gtgaagatga |
| 1141 | gtgaaactga tattactcaa tttcagtctg gacactggct gaatccttcc tctcccctcc |
| 1201 | tcccatccct cataggattt ttcttgtttg gaaaccacgt gttctggttt ccatgatgcc |
| 1261 | catccagtca atctcatgga gggtggagta tggttggagc ctaatcagcg aggtttcttt |
| 1321 | tttttttttt ttcctattgg atcttcctgg agaaaatact tttttttttt ttttttttga |
| 1381 | aacggagtct tgctctgtcg cccaggctgg agtgcagtgg cgcggtcttg gctcactgca |
| 1441 | agctccgtct cccgggttca cgccattctc ctgcctcagc ctcccgagca gctgggacta |
| 1501 | caggcgcccg ccacctcgcc cggctaatat tttgtatttt tagtagagac ggggtttcac |
| 1561 | tgtgttagcc aggatggtct cgatctcctg accttgtgat ccacccgcct cggcctccct |
| 1621 | aacagctggg atttacaggc gtgagccacc gcgccctgcc tagaaaagac attttaataa |
| 1681 | ccttggctgc cgtctctggc tatagataag tagatctaat actagtttgg atatctttag |
| 1741 | ggtttagaat ctaacctcaa gaataagaaa tacaagtaca aattggtgat gaagatgtat |
| 1801 | tcgtattgtt tgggattggg aggctttgct tattttttaa aaactattga ggtaaagggt |
| 1861 | taagctgtaa catacttaat tgatttctta ccgtttttgg ctctgttttg ctatatcccc |
| 1921 | taatttgttg gttgtgctaa tctttgtaga aagaggtctc gtatttgctg catcgtaatg |
| 1981 | acatgagtac tgctttagtt ggtttaagtt caaatgaatg aaacaactat ttttccttta |
| 2041 | gttgatttta ccctgatttc accgagtgtt tcaatgagta aatatacagc ttaaacat |
| SEQ ID NO: 10 | |
| 1 | msvdpacpqs 1pcfeasdck esspmpvicg peenypslqm ssaemphtet vsplpssmdl |
| 61 | liqdspdsst spkgkqptsa eksvakkedk vpvkkqktrt vfsstqlcvl ndrfqrqkyl |
| 121 | slqqmqelsn ilnlsykqvk twfqnqrmks krwqknnwpk nsngvtqkas aptypslyss |
| 181 | yhqgclvnpt gnlpmwsnqt wnnstwsnqt qniqswsnhs wntqtwctqs wnnqawnspf |
| 241 | yncgeeslqs cmqfqpnspa sdleaaleaa geglnviqqt tryfstpqtm dlflnysmnm |
| 301 | qpedv |

In other embodiments, mouse Nanog corresponds to the nucleotide sequence set forth by NCBI reference No. NM_{—}028016, shown below as SEQ ID NO: 11, and the corresponding amino acid sequence set forth by NCBI reference No. NP_{—}082292, shown below as SEQ ID NO: 12.

| SEQ ID NO: 11 | |
|---|---|
| 1 | tctatcgcct tgagccgttg gccttcagat aggctgattt ggttggtgtc ttgctctttc |
| 61 | tgtgggaagg ctgcggctca cttccttctg acttcttgat aattttgcat tagacattta |
| 121 | actcttcttt ctatgatctt tccttctaga cactgagttt tttggttgtt gcctaaaacc |
| 181 | ttttcagaaa tcccttccct cgccatcaca ctgacatgag tgtgggtctt cctggtcccc |
| 241 | acagtttgcc tagttctgag gaagcatcga attctgggaa cgcctcatca atgcctgcag |
| 301 | tttttcatcc cgagaactat tcttgcttac aagggtctgc tactgagatg ctctgcacag |
| 361 | aggctgcctc tcctcgccct tcctctgaag acctgcctct tcaaggcagc cctgattctt |
| 421 | ctaccagtcc caaacaaaag ctctcaagtc ctgaggctga caagggccct gaggaggagg |
| 481 | agaacaaggt ccttgccagg aagcagaaga tgcggactgt gttctctcag gcccagctgt |
| 541 | gtgcactcaa ggacaggttt cagaagcaga agtacctcag cctccagcag atgcaagaac |
| 601 | tctcctccat tctgaacctg agctataagc aggttaagac ctggtttcaa aaccaaagga |
| 661 | tgaagtgcaa gcggtggcag aaaaaccagt ggttgaagac tagcaatggt ctgattcaga |
| 721 | agggctcagc accagtggag tatcccagca tccattgcag ctatccccag ggctatctgg |
| 781 | tgaacgcatc tggaagcctt tccatgtggg gcagccagac ttggaccaac ccaacttgga |
| 841 | gcagccagac ctggaccaac ccaacttgga acaaccagac ctggaccaac ccaacttgga |
| 901 | gcagccaggc ctggaccgct cagtcctgga acggccagcc ttggaatgct gctccgctcc |
| 961 | ataacttcgg ggaggacttt ctgcagcctt acgtacagtt gcagcaaaac ttctctgcca |
| 1021 | gtgatttgga ggtgaatttg gaagccacta gggaaagcca tgcgcatttt agcaccccac |
| 1081 | aagccttgga attattcctg aactactctg tgactccacc aggtgaaata tgagacttac |
| 1141 | gcaacatctg ggcttaaagt cagggcaaag ccaggttcct tccttcttcc aaatattttc |
| 1201 | atattttttt taaagattta tttattcatt atatgtaagt acactgtagc tgtcttcaga |
| 1261 | cactccagaa gagggcgtca gatcttgtta cgtatggttg tgagccacca tgtggttgct |
| 1321 | gggatttgaa ctcctgacct tcggaagagc agtcgg |
| SEQ ID NO: 12 | |
| 1 | msvglpgphs 1psseeasns gnassmpavf hpenysclqg satemlctea asprpssedl |
| 61 | plqgspdsst spkqklsspe adkgpeeeen kvlarkqkmr tvfsqaqlca lkdrfqkqky |
| 121 | lslqqmqels silnlsykqv ktwfqnqrmk ckrwqknqwl ktsngliqkg sapveypsih |
| 181 | csypqgylvn asgslsmwgs qtwtnptwss qtwtnptwnn qtwtnptwss qawtaqswng |
| 241 | qpwnaaplhn fgedflqpyv qlqqnfsasd levnleatre shahfstpqa lelflnysvt |
| 301 | ppgei |

In preferred embodiments, the reprogramming factor is a histone demethylase, for example any one or more of the following:

AOF (LSD1), AOF1 (LSD2), FBXL11 (JHDM1A), Fbxl10 (JHDM1B), FBXL19 (JHDM1C), KIAA1718 (JHDM1D), PHF2 (JHDM1E), PHF8 (JHDM1F), JMJD1A (JHDM2A), JMJD1B (JHDM2B), JMJD1C (JHDM2C), JMJD2A (JHDM3A), JMJD2B (JHDM3B), JMJD2C (JHDM3C), JMJD2D (JHDM3D), RBP2 (JARID1A), PLU1 (JARID1B), SMCX (JARID1C), SMCY (JARID1D), Jumonji (JARID2), UTX (UTX), UTY (UTY), JMJD3 (JMJD3), JMJD4 (JMJD4), JMJD5 (JMJD5), JMJD6 (JMJD6), JMJD7 (JMJD7), JMJD8 (JMJD8).

In certain preferred embodiments, the histone demethylase is Jhdm2a.

In other embodiments, the reprogramming factor is an inhibitory oligonucleotide targeting a histone methyltransferase, example any one or more of the following:

SUV39H1, SUV39H2, G9A (EHMT2), EHMT1, ESET (SETDB1), SETDB2, MLL, MLL2, MLL3, SETD2, NSD1, SMYD2, DOT1L, SETD8, SUV420H1, SUV420H2, EZH2, SETD7, PRDM2, PRMT1, PRMT2, PRMT3, PRMT4, PRMT5, PRMT6, PRMT7, PRMT8, PRMT9, PRMT10, PRMT11, CARM1.

In certain preferred embodiments, the histone methyltransferase is G9A.

Potential agonists and antagonists of an reprogramming factor, in particular a histone demethylase or a methyltransferase, include organic molecules, peptides, peptide mimetics, polypeptides, nucleic acid molecules (e.g., double-stranded RNAs, siRNAs, antisense polynucleotides), and antibodies that bind to a nucleic acid sequence or polypeptide of the invention and thereby inhibit or decrease its activity, or in the case of agonists increase its activity. Small molecules of the invention preferably have a molecular weight below 2,000 daltons, more preferably between 300 and 1,000 daltons, and most preferably between 400 and 700 daltons. It is preferred that these small molecules are organic molecules.

In preferred embodiments, the agent is n inhibitory oligonucleotide, for example a double-stranded RNA (dsRNA), small inhibitory RNA (siRNA), short hairpin RNA (shRNA), or antisense polynucleotides.

In exemplary embodiments, an shRNA is employed that is directed to a histone methyltransferase, and in particular, to G9A. In one example, a preferred shRNA is shown in SEQ ID NO: 11, TGAGAGAGGATGATTCTTA (shRNA-G9a)

The short hairpin sequences can be cloned into a retroviral vector, for example, but not limited to, pUEG, with a non-silencing control. Efficiency of the shRNA can then be confirmed by qRT-PCR.

Reprogrammed somatic cells can be identified by selecting for cells that express an appropriate selectable marker. In other embodiments, reprogrammed somatic cells are assessed for pluripotency characteristics. The presence of pluripotency characteristics indicates that the somatic cells have been reprogrammed to a pluripotent state. In particular embodiments, pluripotency characteristics refers to many characteristics associated with pluripotency, including, for example, the ability to differentiate into all types of cells and an expression pattern distinct for a pluripotent cell, including expression of pluripotency genes, expression of other ES cell markers, or an expression profile known associated with a stem cell molecular signature.

Induced pluripotent stem cells may express any number of pluripotent cell markers, including: alkaline phosphatase (AP); ABCG2; stage specific embryonic antigen-1 (SSEA-1); SSEA-3; SSEA-4; TRA-1-60; TRA-1-81; Tra-2-49/6E; ERas/ECAT5, E-cadherin; .beta.III-tubulin; .alpha.-smooth muscle actin (.alpha.-SMA); fibroblast growth factor 4 (Fgf4), Cripto, Dax1; zinc finger protein 296 (Zfp296); N-acetyltransferase-1 (Nat1); (ES cell associated transcript 1 (ECAT1); ESG1/DPPA5/ECAT2; ECAT3; ECAT6; ECAT7; ECAT8; ECAT9; ECAT10; ECAT15-1; ECAT15-2; Fthl17; Sal14; undifferentiated embryonic cell transcription factor (Utf1); Rex1; p53; G3PDH; telomerase, including TERT; silent X chromosome genes; Dnmt3a; Dnmt3b; TRIM28; F-box containing protein 15 (Fbx15); Nanog/ECAT4; Oct3/4; Sox2; Klf4; c-Myc; Esrrb; TDGF1; GABRB3; Zfp42, FoxD3; GDF3; CYP25A1; developmental pluripotency-associated 2 (DPPA2); T-cell lymphoma breakpoint 1 (Tcl1); DPPA3/Stella; DPPA4; other general markers for pluripotency, etc. Other markers can include Dnmt3L; Sox15; Stat3; Grb2; SV40 Large T Antigen; HPV16 E6; HPV16 E7, .beta-catenin, and Bmi1. Such cells can also be characterized by the down-regulation of markers characteristic of the differentiated cell from which the pluripotent cell is induced. For example, pluripotent stem cells derived from fibroblasts may be characterized by down-regulation of the fibroblast cell marker Thy1 and/or up-regulation of SSEA-1. It is understood that the present invention is not limited to those markers listed herein, and encompasses markers such as cell surface markers, antigens, and other gene products including ESTs, RNA (including microRNAs and antisense RNA), DNA (including genes and cDNAs), and portions thereof.

Differentiation status of cells is a continuous spectrum, with terminally differentiated state at one end of this spectrum and de-differentiated state (pluripotent state) at the other end. Reprogramming, preferably, refers to a process that alters or reverses the differentiation status of a somatic cell, which can be either partially or terminally differentiated. Reprogramming, preferably, includes complete reversion, as well as partial reversion, of the differentiation status of a somatic cell. In preferred embodiments, the term “reprogramming”, as used herein, encompasses any stage of the differentiation status of a cell along the spectrum toward a less-differentiated state. For example, reprogramming includes reversing a multipotent cell back to a pluripotent cell, reversing a terminally differentiated cell back to either a multipotent cell or a pluripotent cell. In one embodiment, reprogramming of a somatic cell turns the somatic cell all the way back to a pluripotent state. In another embodiment, reprogramming of a somatic cell turns the somatic cell back to a multipotent state. The term less-differentiated state is a relative term and includes a completely de-differentiated state and a partially differentiated state, and any state in between.

To assess reprogrammed somatic cells for pluripotency characteristics, the cells may be analyzed for different growth characteristics and ES cell-like morphology. Cells may be injected subcutaneously into immunocompromised SCID mice to induce teratomas (a standard assay for ES cells). ES-like cells can be differentiated into embryoid bodies (another ES specific feature). Moreover, ES-like cells can be differentiated in vitro by adding certain growth factors known to drive differentiation into specific cell types. Self-renewing capacity, marked by induction of telomerase activity, is another pluripotency characteristics that can be monitored. Functional assays of the reprogrammed somatic cells can be performed by introducing them into blastocysts and determine whether the cells are capable of giving rise to all cell types. (see Hogan et al., 2003). If the reprogrammed cells are capable of forming a few cell types of the body, they are multipotent; if the reprogrammed cells are capable of forming all cell types of the body including germ cells, they are pluripotent. Further, pluripotent cells, such as embryonic stem cells, and multipotent cells, such as adult stem cells, are known to have a distinct pattern of global gene expression profile. This distinct pattern has been termed “stem cell molecular signature.” See, for example, Ramalho-Santos et al., Science 298: 597-600 (2002); Ivanova et al., Science 298: 601-604.

### Combination Treatment

Additionally, in any of the methods as described herein, the agents that induce de-differentiation may be used in combination with any agents (e.g. biological agents, synthetic compounds, genes) known in the art that are used for de-differentiation.

For example, any gene that is associated with pluripotency may be used. The expression of a pluripotency gene is typically restricted to pluripotent stem cells, and is crucial for the functional identity of pluripotent stem cells. The transcription factor Oct-4 (also called Pou5f1, Oct-3, Oct3/4) is an example of a pluripotency gene. Oct-4 has been shown to be required for establishing and maintaining the undifferentiated phenotype of ES cells and plays a major role in determining early events in embryogenesis and cellular-differentiation (Nichols et al., 1998, Cell 95:379-391; Niwa et al., 2000, Nature Genet. 24:372-376). Oct-4 is down-regulated as stem cells differentiate into specialised cells. Other exemplary pluripotency genes include Nanog, and Stella (See Chambers et al., 2003, Cell 113: 643-655; Mitsui et al., Cell. 2003, 113(5):631-42; Bortvin et al. Development. 2003, 130(8):1673-80; Saitou et al., Nature. 2002, 418 (6895):293-300.

In one embodiment, a combination of one or more gene products of Oct3/4, Klf4, Sox family, or c-Myc, in combination with any one of a histone demethylase family gene product (for example Jhdm2a) or a Nanog gene product, may be used. Examples of the Oct family gene include, for example, Oct3/4, Oct1A, Oct6, and the like. Oct3/4 is a transcription factor belonging to the POU family, and is reported as a marker of undifferentiated cells (Okamoto et al., Cell 60:461-72, 1990). Oct3/4 is also reported to participate in the maintenance of pluripotency (Nichols et al., Cell 95:379-91, 1998). Examples of the Klf family gene include Klf1, Klf2, Klf4, Klf5 and the like. Klf4 (Kruppel like factor-4) is reported as a tumor repressing factor (Ghaleb et al., Cell Res. 15:92-96, 2005). Examples of the Myc family gene include c-Myc, N-Myc, L-Myc and the like. c-Myc is a transcription control factor involved in differentiation and proliferation of cells (Adhikary & Eilers, Nat. Rev. Mol. Cell. Biol. 6:635-45, 2005), and is also reported to be involved in the maintenance of pluripotency (Cartwright et al., Development 132:885-96, 2005). A Sox family gene may be, for example Sox2. Sox2 is expressed in early development processes and is a gene encoding a transcription factor (Avilion et al., Genes Dev. 17:126-40, 2003). Exemplary NCBI accession numbers are as follows:

Mouse Human Klf1 Kruppel-like factor 1 (erythroid) NM_{—}010635 NM_{—}006563 Klf2 Kruppel-like factor 2 (lung) NM_{—}008452 NM_{—}016270 Klf5 Kruppel-like factor 5 NM_{—}009769 NM_{—}001730 c-Myc myelocytomatosis oncogene NM_{—}010849 NM_{—}002467 N-Myc v-Myc myelocytomatosis viral related oncogene, NM_{—}008709 NM_{—}005378 neuroblastoma derived (avian) L-Myc v-Myc myelocytomatosis viral oncogene NM_{—}008506 NM_{—}005376 homolog 1, lung carcinoma derived (avian) Oct1A POU domain, class 2, transcription factor 1 NM_{—}198934 NM_{—}002697 Oct6 POU domain, class 3, transcription factor 1 NM_{—}011141 NM_{—}002699, Mouse Human Sox1 SRY-box containing gene 1 NM_{—}009233 NM_{—}005986 Sox3 SRY-box containing gene 3 NM_{—}009237 NM_{—}005634 Sox7 SRY-box containing gene 7 NM_{—}011446 NM_{—}031439 Sox15 SRY-box containing gene 15 NM_{—}009235 NM_{—}006942 Sox17 SRY-box containing gene 17 NM_{—}011441 NM_{—}022454 Sox18 SRY-box containing gene 18 NM_{—}009236 NM_{—}018419.

All of these genes are those commonly existing in mammals including human, and for use of the aforementioned gene products in the present invention, genes derived from other mammals (those derived from mammals such as mouse, rat, bovine, ovine, horse, and ape) can be used. In addition to wild-type gene products, mutant gene products including substitution, insertion, and/or deletion of several (for example, 1 to 10, preferably 1 to 6, more preferably 1 to 4, still more preferably 1 to 3, and most preferably 1 or 2) amino acids and having similar function to that of the wild-type gene products can also be used. For example, as a gene product of c-Myc, a stable type product (T58A) may be used as well as the wild-type product.

The method can also include a factor which induces immortalization of cells. For example, the method may include a combination of a factor comprising a gene product of the TERT gene. The method may alternatively include any of the aforementioned gene products in combination with a factor comprising a gene product or gene products of one or more kinds of the following genes: SV40 Large T antigen, HPV16 E6, HPV16 E7, and Bmi1. TERT is essential for the maintenance of the telomere structure at the end of chromosome at the time of DNA replication, and the gene is expressed in stem cells or tumor cells in humans, while it is not expressed in many somatic cells (Horikawa et al., P.N.A S. USA 102:18437-442, 2005). SV40 Large T antigen, HPV16 E6, HPV16 E7, or Bmi1 was reported to induce immortalization of human somatic cells in combination with Large T antigen (Akimov et al., Stem Cells 23:1423-33, 2005; Salmon et al., Mol. Ther. 2:404-14, 2000). The NCBI accession numbers of TERT and Bmi1 genes are as follows:

Mouse Human TERT telomerase reverse transcriptase NM_{—}009354 NM_{—}198253 Bmi1 B lymphoma Mo-MLV NM_{—}007552 NM_{—}005180 insertion region 1.

The present invention further provides transgenic mice comprising the somatic cells of the invention.

### Methods for Monitoring Somatic Cell Fusion and Reprogramming

The invention features methods of monitoring somatic cell fusion comprising contacting a somatic cell comprising a Cre recombinase protein with an embryonic cell, where the embryonic cell comprises a fluorescent Cre recombination excision reporter, and where detection of the fluorescent Cre recombination reporter is used to monitor cell fusion.

The method also includes the step of monitoring somatic cell reprogramming, where the somatic cell comprises GFP, and detection of GFP is used to monitor reprogramming.

In particular embodiments of the invention, Oct4-directs GFP activation in the somatic cell. These somatic cells may be obtained from Oct4-GFP transgenic mice, or may be engineered as described herein. When the cells are obtained from a transgenic mouse, such a transgenic mouse can be produced using standard techniques known in the art and as described herein (see Bronson et al., 1996).

In other embodiments, the somatic cell is further engineered to stably co-express Cre and the puromycin resistance gene.

In exemplary embodiments, the selectable marker, e.g. GPF, is linked to an appropriate endogenous pluripotency gene, e.g. Oct-4, such that the expression of the selectable marker substantially matches the expression of the endogenous pluripotency gene. By “substantially match”, it is meant that the expression of the selectable marker substantially reflects the expression pattern of the endogenous pluripotency gene. In other words, the selectable marker and the endogenous pluripotency gene are co-expressed. For purpose of the present invention, it is not necessary that the expression level of the endogenous gene and the selectable marker is the same or even similar. It is only necessary that the cells in which an endogenous pluripotency gene is activated will also express the selectable marker at a level sufficient to confer a selectable phenotype on the reprogrammed cells. Preferably, in certain exemplary embodiments, the embryonic cell comprises CAG-loxP-LacZ::neomycin-polyA-loxP-DsRed.T3 as the fluorescent Cre recombination excision reporter.

In particular embodiments, fusion or reprogramming can be monitored using fluorescent microscopy or flow cytometry. For example, dual-color flow cytometry is used to quantitatively monitor cell fusion. In other examples, flow cytometry is used to monitor reprogramming frequency, where reprogramming frequency is represented by the ratio of GFP+DsRed+ cells to total DsRed+ cells. Flow cytometry can also be used to monitor reprogramming efficacy, where reprogramming efficacy is represented by the distribution of GFP fluorescence intensity of individual cells from the DsRed+ population. The method can provide a measurement of the efficacy of Oct4-GFP reactivation in somatic cells after fusion.

Screening Methods for Agents that Alter Somatic Cell Fusion

The invention also features methods for identifying agents that alter somatic cell fusion. In preferred examples, the methods comprise contacting a somatic cell comprising a Cre recombinase protein with an embryonic cell, where the embryonic cell comprises a fluorescent Cre recombination excision reporter, and wherein detection of the fluorescent Cre recombination reporter is used to monitor cell fusion; contacting the cells with a candidate agent, wherein detection of the fluorescent Cre recombination reporter is used to identify an agent that alters somatic cell fusion.

In certain embodiments, the method further comprises identifying an agent that alters somatic cell reprogramming comprising the step of monitoring somatic cell reprogramming, wherein the somatic cell comprises GFP and detection of GFP is used to identify an agent that alters somatic cell reprogramming.

In still further embodiments, the cells are contacted with the candidate agent 12, 16, 20, 24, 38, 32, 36, 40, 44, 46, 50, 54, 58 or more hours after cell fusion. Preferably, the cells are contacted 24-48 hours after cell fusion.

In further preferred embodiments, the cells are contacted in the presence of polyethyleneglycol (PEG).

In other exemplary embodiments, the invention features methods of identifying an agent that alters somatic cell fusion and reprogramming comprising contacting a somatic cell comprising a Oct4-GFP Cre recombinase protein with an embryonic cell, wherein the embryonic cell comprises a fluorescent Cre recombination excision reporter, and wherein detection of the fluorescent Cre recombination reporter is used to monitor cell fusion; and contacting the cells with a candidate agent, wherein detection of the fluorescent Cre recombination reporter is used to identify an agent that alters somatic cell fusion and detection of GFP is used to identify an agent that alters somatic cell reprogramming.

In certain examples, fusion or reprogramming is monitored using fluorescent microscopy or flow cytometry, for example dual-color flow cytometry to quantitatively monitor cell fusion.

Flow cytometry can be used to monitor reprogramming frequency, where reprogramming frequency is represented by the ratio of GFP+DsRed+ cells to total DsRed+ cells. In this way, the reprogramming frequency is monitored after treatment with the agent.

Flow cytometry can also be used to monitor reprogramming efficacy, where reprogramming efficacy is represented by the distribution of GFP fluorescence intensity of individual cells from the DsRed+ population. Accordingly, the method provides a measurement of the efficacy of Oct4-GFP reactivation in somatic cells after fusion. In this way, the reprogramming efficacy is monitored after treatment with the agent.

A reprogramming agent may belong to any one of many different categories. For example, the agent may be selected from, but not limited to small molecules, peptides and oligonucleotides.

Candidate agents used in the invention encompass numerous chemical classes, for example organic molecules, including small organic compounds. Candidate agents are also found among biomolecules including peptides, saccharides, fatty acids, steroids, purines, pyrimidines, nucleic acids and derivatives, structural analogs or combinations thereof.

Such candidate agents may be naturally arising, recombinant or designed in the laboratory. The candidate agents may be isolated from microorganisms, animals, or plants, or may be produced recombinantly, or synthesized by chemical methods known in the art. In some embodiments, candidate agents are isolated from libraries of synthetic or natural compounds using the methods of the present invention. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, including acylation, alkylation, esterification, amidification, to produce structural analogs.

There are numerous commercially available compound libraries, including, for example, the Chembridge DIVERSet. Libraries are also available from academic investigators, such as the Diversity set from the NCI developmental therapeutics program.

The screening methods described herein are based on assays performed on cells. These cell-based assays may be performed in a high throughput screening (HTS) format, which has been described in the art. For example, Stockwell et al. described a high-throughput screening of small molecules in miniaturized mammalian cell-based assays involving post-translational modifications (Stockwell et al., 1999). Likewise, Qian et al. described a leukemia cell-based assay for high-throughput screening for anti-cancer agents (Qian et al., 2001). Both references are incorporated herein in their entirety.

As described herein, DNA methylation and histone acetylation are two known events that alter chromatin toward a more closed structure. Potential targets envisioned by the methods of the invention are regulators of epigenetic modification.

As described herein, DNA methylation inhibitors are a class of agents that may be used in the methods of the invention.

In preferred embodiments, the agent inhibits a histone demethylase, for example any one or more of the following:

AOF (LSD1), AOF1 (LSD2), FBXL11 (JHDM1A), Fbxl10 (JHDM1B), FBXL19 (JHDM1C), KIAA1718 (JHDM1D), PHF2 (JHDM1E), PHF8 (JHDM1F), JMJD1A (JHDM2A), JMJD1B (JHDM2B), JMJD1C (JHDM2C), JMJD2A (JHDM3A), JMJD2B (JHDM3B), JMJD2C (JHDM3C), JMJD2D (JHDM3D), RBP2 (JARID1A), PLU1 (JARID1B), SMCX (JARID1C), SMCY (JARID1D), Jumonji (JARID2), UTX (UTX), UTY (UTY), JMJD3 (JMJD3), JMJD4 (JMJD4), JMJD5 (JMJD5), JMJD6 (JMJD6), JMJD7 (JMJD7), JMJD8 (JMJD8).

In certain preferred embodiments, the target histone demethylase is Jhdm2a.

### Cells

The invention includes a reprogrammed cell produced by a method for reprogramming described herein.

The cells can be reprogrammed by treatment with one or more agents, as described herein. In certain examples, the agent is a gene. Accordingly, the present invention provides somatic cells comprising a pluripotency gene, or one or more pluripotency genes. Preferably, these genes belong to the histone methyltransferase or histone demethylase family of enzymes, and in particular G9A and Jdhm2a. In certain embodiment, the gene can be linked to DNA encoding a selectable marker such that the expression of the selectable marker substantially matches the expression of the endogenous pluripotency gene. If two pluripotency genes are expressed, then the somatic cells of the present invention comprise two pluripotency genes, each of which can be linked to DNA encoding a distinct selectable marker.

The pluripotency gene pluripotency gene may be expressed from an inducible promoter. An inducible promoter refers to a promoter that, in the absence of an inducer (such as a chemical and/or biological agent), does not direct expression, or directs low levels of expression of an operably linked gene (including cDNA), and, in response to an inducer, its ability to direct expression is enhanced. For example, a tetracycline-inducible promoter is an example of an inducible promoter that responds to an antibiotics. (Gossen et al., 2003).

### Uses

The present invention provides reprogrammed somatic cells produced by the methods of the invention. The methods described herein can be used for the generation of cells of a desired cell type, and have a wide range of applications, for example, in treating or preventing a condition.

For example, the present invention may encompass a method for stem cell therapy comprising: (1) isolating and collecting a somatic cell from a patient; (2) inducing said somatic cell from the patient into a pluripotent stem cell; (3) inducing differentiation of the pluripotent stem cell, and (4) transplanting the differentiated cell from step (3) into the patient.

### Kits

Also featured in the invention are kits.

Preferably, the kits of the invention feature a reprogrammed somatic cell produced according to any one of the described methods, and instructions for use. The kits of the invention may be used for monitoring somatic cell fusion, where the kits comprise a somatic cell comprising a Cre recombinase protein and an embryonic cell comprising a fluorescent Cre recombination excision reporter, and instructions for use according the methods described herein. In exemplary embodiments, the kits are further used for monitoring cell reprogramming.

In other embodiments, the kit comprises a sterile container which contains the reprogrammed somatic cell produced according to the methods of the invention, or the somatic cell and the agents needed for reprogramming; such containers can be boxes, ampules, bottles, vials, tubes, bags, pouches, blister-packs, or other suitable container form known in the art. Such containers can be made of plastic, glass, laminated paper, metal foil, or other materials suitable for holding nucleic acids. The instructions will generally include information about the use of the agents described herein. In other embodiments, the instructions include at least one of the following: description of the agents; methods for using the enclosed materials for treatment of a condition or a disease. The instructions may be printed directly on the container (when present), or as a label applied to the container, or as a separate sheet, pamphlet, card, or folder supplied in or with the container.

The following examples are offered by way of illustration, not by way of limitation. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

### EXAMPLES

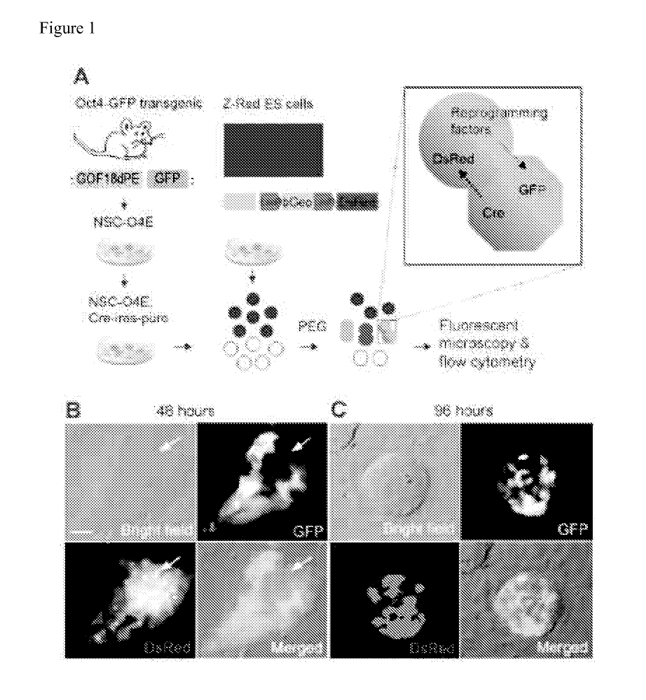

Most of the current reprogramming regimes using ESCs typically involves polyethylene glycol (PEG)-induced cell fusion of ESCs and somatic cells carrying two different drug resistant genes, followed by long-term selection to yield hybrid clones [1 12,14]. The low frequency of cell fusion makes it challenging to immediately identify cells that have undergone fusion. As a consequence, very little is known about the essential process of reprogramming at the early stage. Double drug selection also leads to massive cell death and release of various factors, which may affect the reprogramming process. The experiments described herein are directed at establishing a novel method, termed CLEAR (Cre-LoxP-based, EGFP-inducible Assay for Reprogramming). A combination of live fluorescent microscopy and quantitative flow cytometry allows monitoring early events of ESC fusion-induced reprogramming and quantitative analysis of the frequency and efficacy of re-activating Oct4-GFP expression in adult somatic cells (FIG. 1a). The methods described herein have enabled the identification of a pair of opposing histone-modifying enzymes, the histone H3 lysine 9 (H3K9) methyltransferase G9a and the jumonji-domain containing H3K9 demethylase Jhdm2a [16-18], as epigenetic regulators for ESC fusion-induced Oct4-GFP re-activation during reprogramming. The results described herein, in part, suggest that erasure of epigenetic methylation markers cooperates with transcriptional re-setting to achieve pluripotency.

### Example 1

### Visualization of ESC Fusion-Induced Oct4 Reactivation in Adult NSCs with CLEAR

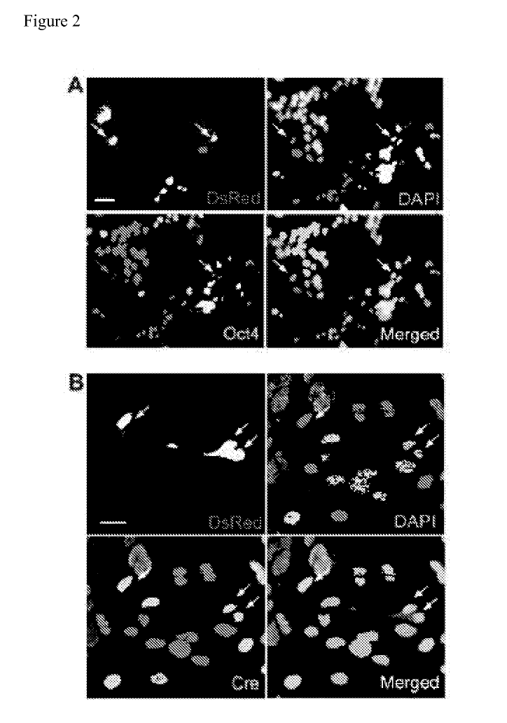

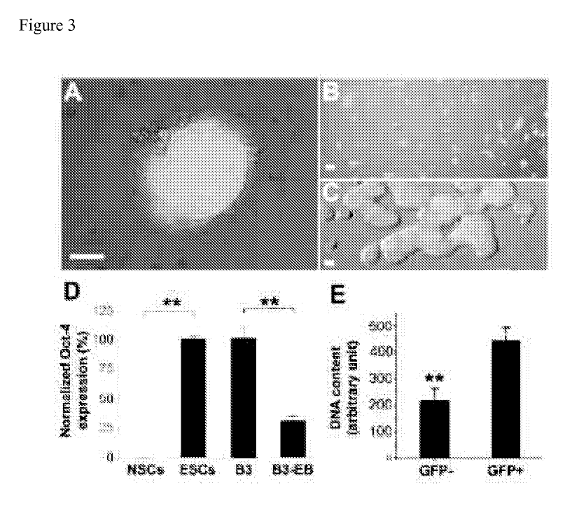

CLEAR strategy uses engineered ESCs and NSCs for monitoring fusion-induced DsRed expression and reprogramming-induced GFP expression (FIG. 1A). Z-Red ESCs were derived from a clonal ESC line containing one copy of a transgene (CAG-loxP-LacZ::neomycin-polyA-loxP-DsRed.T3) as a Cre recombination excision reporter [22]. Upon introduction of Cre activity, transfected cells exhibited strong red fluorescence resulting from the DsRed expression (FIG. 2A). This line of ESCs has been previously used to generate reporter mice and we confirmed that Z-Red ESC were capable of reprogramming somatic NSCs after fusion followed by long-term double drug selection (FIG. 3).

Adult NSCs were isolated from Oct4-GFP (GOF 18-A PE-EGFP) transgenic mice [19,23] and transduced by retroviruses to stably co-express Cre and the puromycin resistance gene through a bicistronic cassette (termed CIPOE NSCs hereafter). Somatic cells with Oct4-GFP transgene integration have been used previously to investigate reprogramming, in which the regulatory elements of Oct4 direct reliable GFP reactivation from somatic genomes in reprogrammed ESC-like hybrid cells [12,14] (also see FIG. 3). Multiple CIPOE NSC lines from Oct4-GFP transgenic mice were established, characterized and used for subsequent cell fusion experiments. Functional Cre activity was demonstrated by the strong nuclear Cre immunoreactivity and effective excision of the LoxP-flanking element after transfection with a reporter plasmid (FIG. 2B).

First, the temporal and spatial resolution of CLEAR strategy in monitoring cell fusion and reprogramming-induced Oct4-GFP expression was examined. PEG 1500 was used to induce cell fusion since the spontaneous fusion rate is considerably low under normal conditions without any selection pressure (data not shown). After induced fusion between Z-Red ESCs and CIPOE NSCs, the emergence of DsRed+ cells at 24 and 48 hours was observed, as shown in a typical mosaic ESC-like colony (FIG. 1B), suggesting that Cre-mediated recombination and subsequent DsRed expression occurred rapidly and allowed early identification of potential reprogramming events in fused cells. Interestingly, while many cells remain GFP− among DsRed+ cells, some GFP− cells were observed at 48 hours, indicating that ESC-induced re-activation of Oct4 expression was initiated within a short period of time. At 96 hours after fusion, majority of DsRed+ cells were GFP+ (FIG. 1C), suggesting that ESC fusion-induced re-activation of Oct4 expression from somatic cells is highly efficient. It was also observed that the GFP+ cells tended to appear first from the outlining border of a colony (FIG. 1B), implicating a spatial order and variegated speed of reprogramming among the fused cell population. These results showed that CLEAR is capable of visualizing the early fused cells and reprogramming-induced Oct4-GFP re-activation simultaneously, and yields both temporal and spatial information on reprogramming.

### Example 2

### Characterization of ESC Fusion-Induced Reprogramming with CLEAR

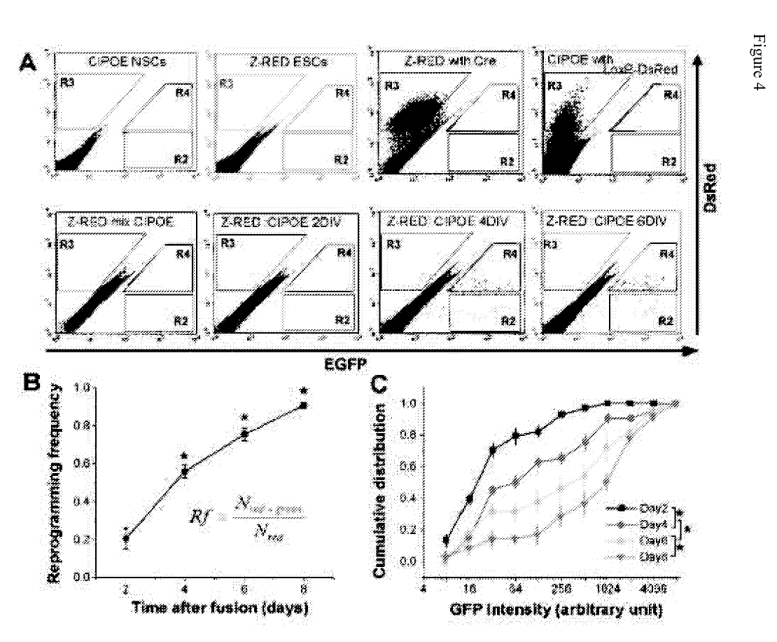

To quantitatively analyze the reprogramming-induced Oct4-GFP re-activation, dual-color flow cytometry was used to display and measure the cell population that underwent PEG-induced cell fusion. Viable cells were identified by their typical FSC (Forward Scatter) and SSC (Side Scatter) properties. To ensure appropriate gating for GYP+ and DsRed+ events and to compensate the spectrum crossover of GFP and DsRed signals, the system was first calibrated using multiple control cells, including Z-Red ESCs, CIPOE NSCs, mixed Z-Red ESCs and CIPOE NSCs without PEG. Z-Red ESCs transfected with a constitutive Cre expression plasmid, and CIPOE NSCs transfected with a Cre excision reporter plasmid (FIG. 4A). The resulting gates allowed accurate quantification of DsRed+GFP+ cells and DsRed+GFP− cells, as shown in typical analytic dot plots (FIG. 4A).

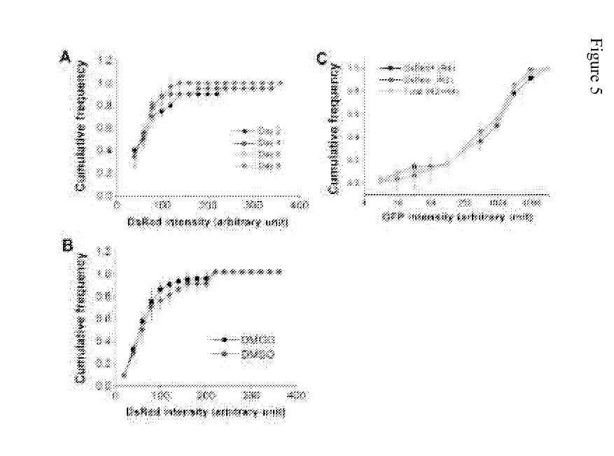

ESC fusion-induced Oct4 re-activation from NSCs was quantitatively measured using two different approaches. In the first approach, the reprogramming frequency (Rf) is determined by the ratio of GFP DsRed+ cells to total DsRed+ cells. Time course analysis showed that over a period of initial 8 days after fusion, the reprogramming frequency increased from 20±5% at day 2 to 90+2% at day 8 (n=4; FIG. 4B). The spontaneous reprogramming frequency in the absence of PEG was below the detection threshold. In the second approach, the reprogramming efficacy is determined by the distribution of GFP fluorescence intensities of individual cells from the DsRed+ population (FIG. 4C). This analysis provides a measurement of the efficiency of Oct4-GFP re-activation in NSCs after successful fusion. It was found that the reprogramming efficacy steadily increased up to 8 days after induction of fusion (FIG. 4C). In contrast, fusion-induced DsRed expression did not significantly change during day 2 and day 8 (FIG. 5A). With these two types of analysis, the CLEAR strategy enables quantification of the reprogramming frequency and efficacy over time, especially at critical early stages after cell fusion.

### Example 3

### Involvement of Chromatin Demethylation in ESC-Induced Oct4 Reactivation in Adult Somatic Stem Cells

To explore the underlying mechanism for reprogramming, the potential involvement of chromatin-modifying enzymes was assessed. A panel of pharmacological inhibitors of histone acetyltransferases, deacetylases, methyltransferases and demethylases was screened during the first 48 hours after fusion. Administration of inhibitors only during early time window after fusion ensures specific effects on reprogramming but not long-term non-specific effects on survival, proliferation and differentiation of hybrid cells. The results showed that the HDAC inhibitor Trichostatin A (TSA) as well as various other inhibitors were either ineffective, toxic to the cells, or led to mild deficit on reprogramming-induced Oct4 reactivation (see Table 1). Table 1, shown below, shows the effects of pharmacological inhibitors on reprogramming.

TABLE 1

| | | Concen- | |
|---|---|---|---|
| Inhibitors | Target | tration | Effects |
| TSA | HDAC | 100 nM | pro-differentiation; toxic |
| Anacardic | CBP/p300 | 5 μM | mild reprogramming deficit; |
| acid | | | toxic |
| DMOG | dioxygenase | 5 μM | strong reprogramming deficit |
| Tranylcy- | LSD1 | 2 μM | mild reprogramming deficit |
| promine | | | |
| Azt | DNMT | 2 μM | anti-proliferative; mild |
| | | | toxic |

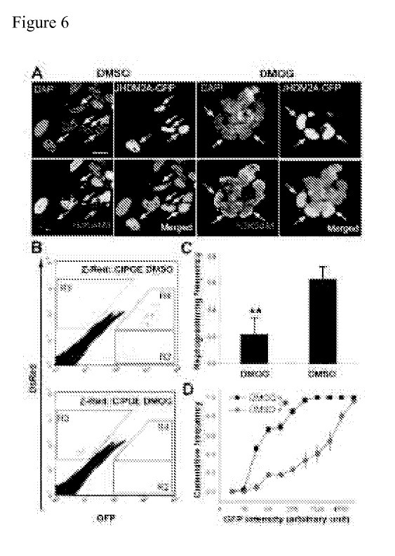

In contrast, dimethyloxalylglycine (DMOG), an inhibitor of Fe2+ and 2-oxoglutarate dependent dioxygenases [24,25], including the AlkB family of DNA repair demethylases and jumonji family of histone demethylases [26-28], considerably reduced ESC-induced Oct4 re-activation in NSCs. To confirm the blocking effects of DMOG on histone demethylases, an immuno labeling assay previously developed for JHDM2A was used. Overexpression of Jhdm2a in heterologous cell lines led to dramatic loss of H3K9 dimethylation, which was blocked by the DMOG treatment (10 μM FIG. 6A). CLEAR analysis showed that treatment with DMOG, but not DMSO, resulted in significant decreases in both the reprogramming frequency and efficacy of ESC-induced Oct4-GFP re-activation in NSCs (FIGS. 6B, 6C, 6D). In contrast, the Cre-mediated DsRed expression remained unchanged when compared with the control DMSO treatment (FIG. 5B). These experiments indicate that activities of dioxygenases, including chromatin-associated histone demethylases or putative DNA demethylases, are important for ESC-induced reprogramming of the adult NSCs.

### Example 4

### Regulation of ESC-Induced Oct4 Reactivation in Adult NSCs by G9a and Jhdm2a

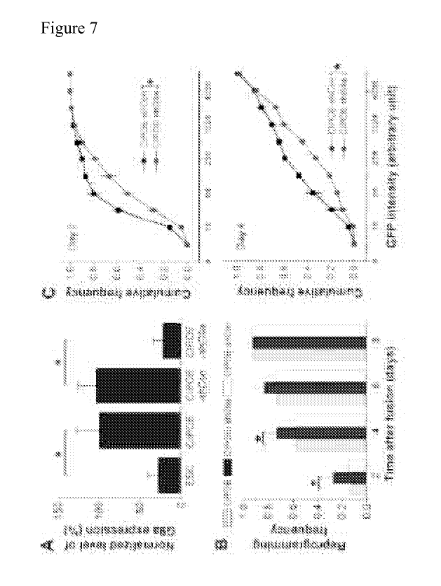

Next, the molecular identities of epigenetic factors that play critical roles in reprogramming were examined. Previous studies suggest that in somatic cells H3K9 methylation near the Oct4 promoter region is mediated by enchroinatin-specific histone methyltransferase G9a [16]. It was found that G9a expression is considerably higher in the somatic CIPOE cells than that in ESCs (FIG. 7A). Since ESC-induced re-activation of Oct4 is accompanied with hi stone and DNA demethylation during reprogramming, it was examined whether the H3K9-specific methyltransferase G9a may antagonize reprogramming. Using a previously validated small short-hairpin RNA (shRNA) for mouse G9a [29], the expression of endogenous G9a was knocked down in CIPOE cells, as shown by quantitative real-time PCR (FIG. 7A). Interestingly, expression of shRNA-G9a, but not control shRNA, in stable lines of CIPOE NSCs accelerated the speed of ESC-induced Oct4-GFP expression, as shown by 110% and 26% increase in the reprogramming frequency at day 2 and day 4, respectively (FIG. 7C). The efficacy of ESC-induced Oct4-GFP re-activation in NSCs was also significantly enhanced at day 2 and day 4 (FIG. 7C). These results suggest that H3K9 methyltransferase G9a constrains ESC-induced Oct4 re-activation during early phases of reprogramming in somatic cells.

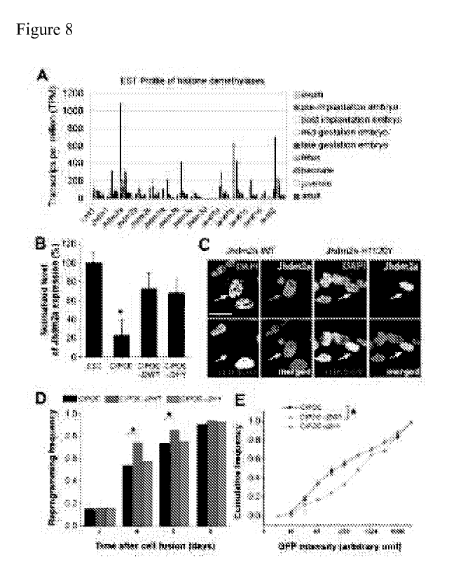

Dynamic histone methylation may result from opposing actions of histone methyltransferases and demethylases [26] Given the preliminary findings from pharmacological analysis (FIG. 6), a next set of experiments sought to identify the histone demethylase that may promote the reprogramming process. The gene expression of currently identified histone demethylases was surveyed through expressed sequence tag (EST) counts at different developmental stages in various tissues. It was found that Jhdm2a is highly expressed in the early mouse embryo and is particularly abundant in the ovum, which is known to be enriched with reprogramming activities (FIG. 8A). Further analysis with quantitative real-time PCR showed that the expression level of Jhdm2a was four times higher in ESCs than that in adult NSCs (FIG. 8B). To examine a potential role of Jhdm2a in reprogramming, Jhdm2a was over-expressed in CIPOE NSCs and it was confirmed that over-expression of Jhdm2a, but not an enzymatically inactive mutant Jhdm2a-H1120Y, induced genome-wide loss of H3K9 dimethylation (FIG. 8C). CLEAR analysis showed that over-expression of wild-type Jhdm2a in CIPOE NSCs increased the frequency of ESC-induced Oct4-GFP re-activation by 36% at day 4, but not at day 2 as in the case of G9a knockdown (FIG. 8D). The efficacy of ESC-induced Oct4-GFP re-activation was also significantly enhanced at day 4 (FIG. 8E). In contrast, over-expression of a mutant Jhdm2a (H 1120Y) exhibited no detectable effects on reprogramming frequency and efficacy (FIGS. 8D, 8E), supporting that the catalytic H3K9 demethylation activity of Jhdm2a is important in promoting ESC-induced re-activation of Oct4 expression in adult NSCs.

Taken together, these results suggest that H3K9 demethylation mediated by the coordinated actions between Jhdm2a and G9a regulate ESC-induced reactivation of Oct4-GFP expression in adult NSCs.

### Example 5

### Nanog Enhances Effects of Jhdm2a on Oct4-GFP Reactivation in Somatic Stem Cells

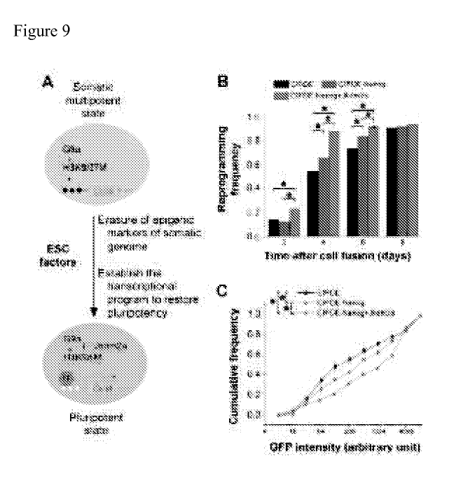

Previous studies have shown that long-term expression of the pluripotency gene Nanog promotes ESC fusion-induced reprogramming and suggested that Nanog may collaborate with unknown epigenetic regulators to facilitate reprogramming [8] (FIG. 9A). CLEAR analysis showed that over-expression of Nanog in CIPOE NSCs substantially increased the reprogramming frequency at day 4 (FIG. 9B). The reprogramming efficacy was also significantly enhanced with Nanog over-expression at day 4 (FIG. 9C). These results suggest that short-term Nanog over-expression accelerates ESC-induced Oct4-GFP reactivation in adult somatic cells and are consistent with previous findings on long-term effects of Nanog expression [8]

To test whether Jhdm2a-mediated H3K9 demethylation constitutes one of epigenetic modification activities in coordination with action of ESC-specific transcription factors (FIG. 9A), both Jhdma2a and Nanog were co-expressed in CIPOE NSCs and reprogramming was examined based on CLEAR analysis. Interestingly, such combination led to further enhancement of reprogramming frequency and efficacy in a time-window ranging from day 2 to day 6 (FIGS. 9B, 9C).

### Example 6

### DNA Demethylation of Oct4 Promoter Regions and Partial Reactivation of Endogenous Oct4 Expression by Knockdown of G9a in Adult NSCs

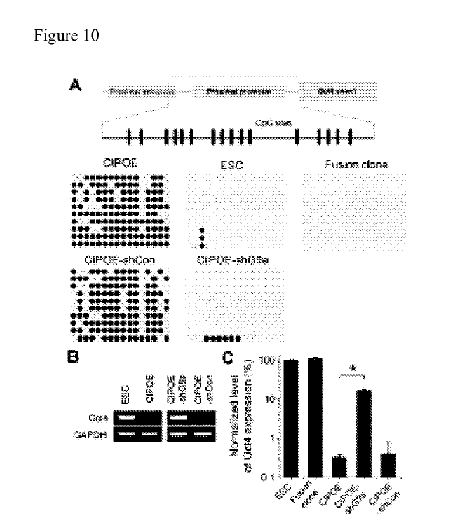

Reprogramming requires erasure of the somatic epigenoine including both histone and DNA modification [13,30,31]. Next it was further tested whether DNA demethylation of pluripotency gene Oct4 induced by ESC-mediated reprogramming may partially account for the facilitating effects of histone demethylation during reprogramming. Extensive bisulfite sequencing revealed that ESC fusion dramatically reduced DNA methylation in Oct4 promoter regions, as compared to that in CIPOE NSCs (FIG. 10A). Surprisingly, independent of cell fusion, CIPOE NSCs expressing shRNA against G9a, but not a control shRNA, exhibit considerably decreased DNA methylation in Oct4 promoter regions with levels very similar to those in Z-Red ESCs or reprogrammed hybrid clones (FIG. 10A). Despite the transgenic nature of CIPOE NSCs, bisulfite primers were designed to span part of the Oct4 coding exon thus the observed demethylation directly reflects the endogenous promoter status. The impact of G9a knockdown on endogenous Oct4 expression was also evaluated. Interestingly, the expression of endogenous Oct4 became partially re-activated in adult NSCs expressing shRNA against G9a as shown by both conventional (FIG. 10B) and quantitative PCR (FIG. 10C). The mRNA abundance of Oct4 expression measured in bulk adult NSC cultures with G9a knocking-down reached around 10% of that in ESCs, while little expression was detected in CIPOE or CIPOE cells expressing the control shRNA. Taken together, these results suggest that G9a is critical to impose epigenetic silencing machinery on Oct4 through maintaining DNA methylation in adult NSCs, while removing G9a induces either active or passive DNA demethylation [32-34] that then relieves epigenetic silencing and facilitates ESC-induced reprogramming.

Rapid advances in stem cell biology have created fascinating possibilities to reprogram somatic nuclei for therapeutic applications [3,35]. Mechanistic understanding of reprogramming will likely be benefited from studies on a variety of reprogramming paradigms including SCNT, cell fusion, purified protein extracts, and genetic manipulation using defined factors. Based on the cell fusion paradigm, CLEAR enables direct and independent visualization of rare fusion and transient reprogramming events at the single cell level. This sensitive method allows quantitative analysis of ESC fusion-induced Oct4 re-activation during initial stages of reprogramming of adult somatic stem cells, especially the tempo regulation. The results present herein demonstrate in part that cell fusion does not necessarily guarantee reprogramming, and on average it takes at least 4 days for reprogramming to complete. Within an ESC-like fusion colony, the reprogramming speed is heterogeneous. CLEAR also employs the analytic power of dual-color flow cytometry for quantification of both reprogramming frequency and efficacy. The results presented herein demonstrate, at least in part, that cell fusion also induced a subset of GFP but DsRed− cells, possibly caused by inefficient recombination due to heterogeneous levels of Cre expression. Nevertheless, the accurate analysis of Oct4 reactivation was not compromised, since only successfully fused DsRed− cells were taken into consideration.

As shown herein, the reprogramming efficacy of DsRed+ cells is representative of the total cell population (FIG. 5C). The remarkable reversibility of cellular differentiation has been first demonstrated in amphibian, and recently in mammalian cells [2,3,36]. These SCNT experiments suggest that the somatic epigenome requires extensive reprogramming in order to achieve toti- or pluripotency. However, there is increasing evidence that epigenetic reprogramming is heterogeneous and severely deficient in some cloned embryos. Indeed, it has been shown that H3K9 and associated DNA hypermethylation are closely correlated with restricted developmental potential in cloned embryos [15]. Corroborating these studies, the results herein demonstrate, at least in part, that H3K9 and DNA methylation restrict somatic cell reprogramming by ESCs. Taken together, these results point to important roles of dynamic demethylation for efficient reprogramming by both SCNT and fusion-induced reprogramming. By over-expression of Jhdm2a, genome-wide H3K9 demethylation is observed, and in parallel, knockdown of G9a induced DNA demethylation at the Oct4 promoter. These epigenetic erasure activities may represent critical initial process of reprogramming, which is coupled with re-establishment of pluripotency-specific transcriptional programs mediated by a cohort of transcriptional factors, such as Nanog (FIG. 9A). Recent exciting advances using candidate approaches have identified defined factors that are sufficient to reprogram fibroblasts into pluripotent ESC-like cells with epigenome highly similar to that of normal ESCs [4-6,37,38]. Because reprogramming using these defined factors is achieved through long-term cell growth in culture and with low efficiency, mechanisms underlying reprogramming largely remain as a black box. Complementary to studies on these defined factors, we show that ESC fusion-induced Oct4 reactivation occurs within a rather short period of time (2-4 days) and at high efficiency (up to 95% of all fused cells). In addition, the results suggest that the early phase of reprogramming may critically involve extensive epigenetic remodeling, and transcription factors may play long-term roles in both recruiting epigenetic regulators and stabilizing the pluripotent epigenome (FIG. 9A). The Oct4 transgenic promoter in the system described herein includes the distal enhancer without the proximal enhancer, which ensures highly specific and appropriate level of Oct-4 expression in undifferentiated pluripotent tissue to be reported. It has been shown that Jhdm2a regulates self-renewal of ESCs, while G9a plays critical roles in silencing Oct4 during differentiation of ESCs and differentiating G9a−/− ESCs have a higher probability to revert back to an initial ESC state [16,39]. The bisulfite sequencing analysis suggests that G9a may be directly associated with DNA methylation of Oct4 promoter, although the full activation of the promoter may depend on pluripotency-specific transcription factors. Nevertheless, effects of G9a and Jhdma2a on ESC fusion-induced reprogramming are distinct compared with that of Nanog, especially at initial days of reprogramming (FIGS. 7B, 8D, 9B), highlighting the relative importance of epigenetic and genetic regulation in early phases of reprogramming. Considering that H3K9 dimethylation constitutes only one of many histone modifications, it remains likely other histone demethylases or methyltransferases may also be critically involved in regulation of reprogramming.

DNA and histone modification-mediated epigenetic reprogramming has long been postulated to be essential at stages when developmental potency of cells changes such as during SCNT and fusion with ESCs, yet experimental evidence for the role of specific enzymes is scant. Using the newly developed quantitative system CLEAR, here it has been shown that a pair of histone-modifying enzymes, G9a and Jhdm2a, are epigenetic regulators for Oct4-GFP re-activation during ESC-induced reprogramming. The mechanistic findings described herein may explain the low efficiency of currently adopted reprogramming regime and thus may guide more efficient reprogramming using defined factors or chemicals in the near future. For example, recent chemical screens have identified a biologically active G9a inhibitor that could be very useful in reprogramming somatic cells [40]. The CLEAR system may also aid identifying additional reprogramming factors [7] and facilitating molecular understanding of how a genome is reprogrammed, and ultimately will advance efforts to engineer developmental potentials of somatic cells for therapeutic applications.

### Materials and Methods

The Examples described herein were performed using, but not limited to, the following materials and methods.

### Constructs and Transfection

Turbo Cre cDNA was cloned into the MSCV retroviral vector modified to contain a puromycin resistant gene under the control of TRES. pCAGT-bGeo-LoxP Cre excision reporter plasmid was made by cloning PCR amplified bGeo-LoxP fragments into pCAG-tdTomato/DsRed vectors. All clones were confirmed by sequencing. The JHDM2A-GFP fusion construct was made by cloning CMV-EGFP (Clontech) fragments into the NdeI and Kpni sites of pcDNA3-11114DM2a. Recombinant DNA research was according to the National Institutes of Health guidelines.

Transfections on ESCs, NSCs, and 293T cells are performed by Amaxa Nucleofection. Typically, 2-54 g DNA is mixed with 5-10 million cells and electroporated using programs (A13 for ESCs, A-31 for NSCs and A-23 for 293T) optimized to achieve high transfection efficiency and low toxicity.

### Isolation and Establishment of Cre-Expressing NSC Lines

Adult NSCs were derived from either hippocampus or subventricular zone of 4-6 week old Oct4-GFP reporter mice as previously described [19]. Specifically, dissected tissues were enzymatically dissociated and a Percoll gradient was applied to isolate a low-buoyancy fraction. Harvested cells were washed, and plated onto plastic dishes in DMEM/F12 medium supplemented with FGF-2 (20 ng/ml), heparin (5 i.tg/rn1) and EGF (20 ng/ml). NSC cultures were maintained in monolayer and passaged once they reach confluency. Engineered retrovirus co-expressing Cre and the puromycin resistant gene were produced and used for infection of NSCs as previously described [20,21]. Briefly, retroviruses were produced through co-transfection of the vector and envelope plasmid VSVG in 293-GP packaging cell lines. Pools of supernatant were harvested and viruses were concentrated by ultracentrifugation at 25,000 rpm for 1.5 hours. Aliquots of viruses were applied to proliferating NSC culture for 12-16 hours. NSC were selected with 1 mg/ml puromycin for at least one week and resistant clones were expanded and verified by immunohistochemistry and western blot for target gene expression.

### PEG-Induced Cell Fusion

The protocol was optimized for PEG-induced cell fusion between ESCs and NSCs to achieve maximum efficiency and minimal toxicity. Equal numbers of ESCs and NSCs were mixed thoroughly and spun down in PBS. The pellet was loosened by gentle tapping and 50% PEG (500 μl for 1×10⁷ cells) was added to the cells continuously over one minute while swirling the mixture in 37° C. water bath Next, 2 ml of ESC medium was layered on top of PEG-cell mixture and one-minute incubation in 37° C., followed by low-speed centrifugation at 1,800 rpm for 5 minutes. After removing the supernatant, the pellet was incubated with ESC medium for one minute, washed, resuspended and plated onto gelatin-coated dishes and grown in Dubelcco's Modified Eagle's Medium, 15% fetal bovine serum supplemented with mouse leukemia inhibitory factor (ESGRO), 0.1 mM nonessential amino acids, 0.1 mM β-mercaptoethanol, and 50 U/ml penicillin/50 μg/ml streptomycin (ESC medium). Based on the CLEAR system, the cell fusion efficiency (DsRed+ cell/total number of cells) is estimated to be 0.34+0.06% under standard condition. The following pharmacological inhibitors were applied in ESC-fusion experiments only during the first 48 hours after fusion. DMOG (5-10 μM; BioMol), Anacardic acid (5-10 μM; Calbiochem), Trichostatin A (TSA, 100 nM-1 μM; Sigma) and azidothymidine (AZT, 1-5 μM; Sigma).

### Microscopy and Flow Cytometry

Live images were taken from Zeiss Axiovert 200M inverted microscope through different optical filters. In dual-color flow cytometry, FACSCalibur system was set up to ensure proper display of 4 parameters: forward and side scatterings as FL1 and 2, GFP and DsRed in FL3 and 4, respectively. Multiple control cells were used to compensate signals emitted from FL3 and FL4, followed by careful gate settings to isolate GFP−DsRed (R3), GFP DsRed− (R2) and GFP−DsRed+ cells (R4).

### Immunocytochemistry

Cultures are fixed with 4% paraformaldehyde (PFA) in 0.1 mM TBS, and blocked in TBS++ (0.1 mM TBS, 5% donkey serum, 0.25% Triton X-100) for 1 hr, and incubated with primary antibodies in TBS++ overnight at 4° C., and rinsed. The following antibodies were used: rabbit anti-H3K9me2 (1:500; Upstate); rabbit anti-GFP (1:500; Molecular Probes), mouse monoclonal anti-Cre (1 1000; Sigma), rabbit anti-DsRed (1 1000; Clontech), mouse monoclonal anti-Oct4 (1 100; Santa Cruz), mouse or rabbit IgG isotpe control (Santa Cruz). After incubation with fluorescently labeled secondary antibodies (1:250; Jackson Immunoresearch) for 90 min at room temperature, cultures are rinsed, stained with 4′,6-diamidino-2-phenylindole (DAPI), rinsed, mounted and stored at 4° C. Images were taken with confocal microscopy system (Zeiss LSMS 10) using multi-track configuration.

shRNA-Mediated Knockdown and Real-Time PCR

The following short hairpin sequences were cloned into a retroviral vector pUEG (Ge et al. 2006): TGAGAGAGGATGATTCTTA (shRNA-G9a); TTCTCCGAACGTGTCACGT (shRNA-non silencing control). Efficiency of the shRNAs was confirmed by qRT-PCR.

For real-time quantitative PCR, total RNAs were purified using RNAeasy kit (Qiagen) and converted to cDNA by SuperScript III (Invitrogen). Triplicate cDNA samples were added to a SYBR-green based quantitative PCR reaction mix and analyzed using the ddCt methods.

β-Actin serves as an internal control for normalization. The primers for G9a, Jhdm2a, Oct4 and β-Actin: CAACTTCCAGAGCGACCAG (G9a forward), ACCTCCAGGTGGTTGTTCAC (G9a reverse), GAAGGCTTCTTAACACCAAACAA (Jhdm2a forward), CATTTGACAGAAGTGGTCTCCA (Jhdm2a reverse), CAGAAGGGCAAAAGATCAAGTAT (Oct4 reverse), CAGTTTGAATGCATGGGAGA (Oct4 forward), TCAACACCCCAGCCATGTA (Actin forward), CAGGTCCAGACGCAGGAT (Actin reverse).

### Bisulfite Genomic Sequencing

For bisulfite genomic sequencing, 500 ng of genomic DNA from each sample was digested by EcoRI overnight, followed by boiling for 5 min and incubation in 0.3 M NaOH at 50° C. for 15 min. Denatured DNA was then embedded in seven 0.67% (w/v) low-melting point agarose beads and treated with a mixture of 2.5 M sodium bisulfite, 0.4 M NaOH, and 0A3 M hydroquinone at 50° C. overnight. Beads were then washed with TE buffer and treated with 0.2 M NaOH for 30 min, followed by washing with TE buffer for 30 min. Prior to PCR amplification, beads were washed with H2O for 30 min. Fresh PCR products were cloned by TA cloning method and sequenced. Efficiency of bisulfite conversion was monitored by the presence of unconverted C residues in non-CpG regions, which were only seldom seen. The primers used for the Oct4 promoter and enhancer region (see FIG. 7A) are 5′-TGGGTTGAAATATTGGGTTTATTT-3′ and 5′-CTAAAACCAAATATCCAACCATA-3′ These primers were designed to span part of the Oct4 coding exon thus directly reflects the endogenous promoter status.

All publications and patent documents cited in this application are incorporated by reference in their entirety for all purposes to the same extent as if each individual publication or patent document were so individually denoted. By their citation of various references in this document, Applicants do not admit any particular reference is “prior art” to their invention.

The following specific references, also incorporated by reference, are indicated above by corresponding reference number.
1. Cowan C A, Atienza J, Melton D A, Eggan K: Nuclear reprogramming of somatic cells after fusion with human embryonic stem cells. Science 2005; 309:1369-1373.
2. Gordon J13: From nuclear transfer to nuclear reprogramming: the reversal of cell differentiation. An nu Rev Cell Dcv Biol 2006; 22:1-22.
3. Hochedlinger K, Jaenisch R: Nuclear reprogramming and pluripotency. Nature 2006; 441.1061-1067.
4. Yu J. Vodyanik M A, Smuga-Otto K, Antosiewicz-Bourget J, Franc J L, Tian S, Nie J. Jonsdoftir G A, Ruotti V, Stewart R., Slukvin, I I, Thomson J A: Induced pluripotent stem cell lines derived from human somatic cells. Science 2007; 318:1917-1920.
5. Takahashi K, Yamanaka S: Induction of pluripotent stem cells from mouse embryonic and adult fibroblast cultures by defined factors. Cell 2006; 126:663-676.
6. Park 1H, Zhao R, West J A, Yabuuchi A, Huo H, Ince T A, Lerou P H, Lensch M W, Daley G Q: Reprogramming of human somatic cells to pluripotency with defined factors. Nature 2008; 451.141-146.
7. Wong C C, Gaspar-Maia A, Ramalho-Santos M, Reijo Pera R A: High-efficiency stein cell fusion-mediated assay reveals Sa114 as an enhancer of reprogramming. PLoS ONE 2008:3:e1955.
8. Silva J, Chambers 1, Pollard S, Smith A: Nanog promotes transfer of pluripotency after cell fusion. Nature 2006; 441:997-1001.
9. Pesce M, Scholer H R. Oct-4: gatekeeper in the beginnings of mammalian development. Stem Cells 2001; 19:271-278.
10. Pan G J, Chang Z Y, Scholer H R, Pei D: Stem cell pluripotency and transcription factor Oct4. Cell Res 2002; 12:321-329.
11. Hattori N, Nishino Y, Ko Y G, Oligane J. Tanaka S, Shiota K. Epigenetic control of mouse Oct-4 gene expression in embryonic stein cells and trophoblast stem cells. J Biol Chem 2004; 279:17063-17069.
12. Do J T, Scholer H R. Nuclei of embryonic stem cells reprogram somatic cells. Stein Cells 2004; 22:941-949.
13. Simonsson S G J: DNA demethylation is necessary for the epigenetic reprogramming of somatic cell nuclei. Nat Cell Biol 2004; 6: 10.
14. Tada M, Takahama Y, Abe K, Nakatsuji N, Tada T: Nuclear reprogramming of somatic cells by in vitro hybridization with ES cells. Cuff Biol 2001; 11:1553-1558.
15. Santos F, Zakhartchenko V, Stojkovic M, Peters A, Jenuwein T, Wolf E, Reik W, Dean W: Epigenetic marking correlates with developmental potential in cloned bovine preimplantation embryos. Cuff Biol 2003; 13:1116-1121.
16. Feldman N, Gerson A, Fang J, Li E, Mang Y, Shinkai Y, Cedar H. Bergman Y: G9a-mediated irreversible epigenetic inactivation of Oct-3/4 during early embryogenesis. Nat Cell Biol 2006; 8:188-194.
17. Tachibana. M, Sugimoto K. Nozaki M, Ueda J, Ohta T Oliki M, Fukuda M, Takeda N, Nikki H, Kato H, Shinkai Y: G9a histone methyltransferase plays a dominant role in euchromatic histone H3 lysine 9 methylation and is essential for early embryogenesis. Genes Dev 2002; 16:1779-1791
18. Yamane K. Toumazou C. Tsukada Y, Erdjument-Bromage H, Tempst P, Wong J, Zlumg Y: JHDM2A, a. JmjC-containing H3K9 demethylase, facilitates transcription activation by androgen receptor. Cell 2006; 125:483-495.
19. Song H, Stevens C F, Gage F H: Astroglia induce neurogenesis from adult neural stem cells. Nature 2002; 417:39-44.
20. Ge 5, Goh E L, Sailor K A, Kitabatake Y. Ming G L, Song H: GABA regulates synaptic integration of newly generated neurons in the adult brain. Nature 2006; 439:589-593.
21. Duan X, Chang J H, Ge S. Faulkner R L, Kim J Y, Kitabatake Y, Litt X13, Yang C H, Jordan J D, Ma D K, Litt C Y, Ganesan S, Chang 14.1. Ming G L, La B. Song H: Disrupted-hi-Schizophrenia 1 regulates integration of newly generated neurons in the adult brain. Cell 2007; 130:1146-1158.
22. Vintersten K. Monetti C. Gertsenstein M. Zhang P, Laszlo L, Biechele S, Nagy A. Mouse in red: red fluorescent protein expression in mouse ES cells, embryos, and adult animals. Genesis 2004; 40:241-246.
23. Ycom V I, Fuhrmarm G, ° vitt C E, Brehm A, Ohbo K, Gross M, Hubner K, Scholer H R: Germline regulatory element of Oct-4 specific for the totipotent cycle of embryonal cells. Development 1996; 122:881-894.
24. Hausinger R P: Fel lialpha.-keloglutarate-dependent hydroxyla.ses and related enzymes. Oil Rev Biochem Mol Biol 2004; 39:21-68.
25. Chen H, Ke Q, Kluz T, Yan Y, Costa M: Nickel ions increase histone H3 lysine 9 dimeklation and induce transgene silencing. Mol Cell Biol 2006; 26:3728-3737.
26. Klose R J, Zhang Y: Regulation of histone methylation by demethylimination and demethylation. Nat Rev Mol Cell Biol 2007; 8:307-318.
27. Falnes P O, Klungland A, Alscili I Repair of methyl lesions in DNA and RNA by oxidative demethylation. Neuroscience 2007; 145:1222-1232.
28. Sedgwick B: Repairing DNA-methylation damage. Nat Rev Mol Cell Biol 2004; 5:148-157.
29. Lee D Y, Northrop J P, Kuo M H, Stallcup M R Historic H3 lysine 9 methyltransferase G9a is a transcriptional coactivator for nuclear receptors. J Biol Chem 2006; 281:8476-8485.
30. Jenuwein T, Allis C D: Translating the histone code. Science 2001; 293:1074-1080.
31. Kimura H, Tada M, Nakatsuji N, Tada T: Histone code modifications on pluripotential nuclei of reprogrammed somatic cells. Mol Cell Bio 12004; 24:5710-5720.
32. Bird A: Perceptions of epigenetics. Nature 2007; 447:396-398.
33. Barreto G, Schafer A, Marhold J, Stack D, Swaminathan S K, Handa V, Doderlein G, Maltry N, Wu W, Lyko F, Niehrs C: Gadd45a promotes epigenetic gene activation by repair-mediated DNA demethylation Nature 2007; 445:671-675.
34. Esteve P O. Chin H G, Smallwood A, Fechery G R, Gangisetty 0, Karpf A R, Carey M F, Pradhan S: Direct interaction between DNMT1 and G9a coordinates DNA and histone methylation during replication. Genes Dev 2006; 20:3089-3103.
35. Pomerantz J, Blau H M: Nuclear reprogramming: a key to stem cell function in regenerative medicine. Nat Cell Biol 2004; 6:810-816.
36. Gurdon J B, Elsdale T R, Fischberg M: Sexually mature individuals of Xenopus laevis from the transplantation of single somatic nuclei. Nature 1958; 182:64-65.
37. Maherali. R S, Wei Xic, Jochen titikaL Sarah Eminli, Katrin Arnold.1 Matthias Stadifeld.1 Robin Yachechko,3 Jason Ichieu,3 Rudolf Jaenisch,5 Kathrin Plath,3,4, and Konrad Hochedlinger Directly Reprogrammed Fibroblasts Show Global Epigenetic Remodeling and Widespread Tissue Contribution. Cell Stem Cell 2007; 1:55-70.
38. Wernig M, Meissner A, Foreman R. Brambrink T, Ku 1V1 HochecRinger K, Bernstein B E, Jaenisch R. In vitro reprogramming of fibroblasts into a pluripotent ES-cell-like state. Nature 2007.
39. Loh V H, Mang W. Chen X, George J, Ng H H: inijd1a. and Jmjd2c historic H3 Lys 9 demethylase regulate self-renewal in embryonic stem cells. Genes Dery 2007; 21:2545-2557.
40. Kubicek S. O'Sullivan R J, August E M, Hickey E R, Zliang Q, Teodoro M L, Rea S, Mechtler K, Kowalski J A, Homon C A, Kelly T A, Jenuwein T. Reversal of H3K9me2 by a small-molecule inhibitor for the G9a historic methyl transferase. Mol Cell 2007; 25:473-481.

1. A method for reprogramming one or more somatic cells comprising:
treating the cells with one or more agents that induces de-differentiation, wherein the agent is selected from a histone methyltransferase inhibitor or a histone demethylase activator,
thereby generating a reprogrammed cell.

2. A method for reprogramming one or more somatic cells comprising:
treating the cells with one or more agents that induces de-differentiation; and
detecting the expression of one or more markers, where at least one marker indicates cell reprogramming;
selecting a cell that expresses the one or more markers;
thereby generating a reprogrammed cell.

3. The method of claim 1, further comprising contacting a somatic cell with an embryonic stem cell.

4. The method of claim 1, wherein the somatic cell comprises a Cre recombinase protein.

5. The method of claim 3, wherein the embryonic cell comprises a fluorescent Cre recombination excision reporter, and wherein detection of the fluorescent Cre recombination reporter is used to monitor cell fusion.

6. The method of claim 4, wherein the somatic cell further comprises GFP and detection of GFP is used to identify an agent that alters somatic cell reprogramming.

7. The method of claim 3, wherein the cells are contacted in the presence of polyethyleneglycol (PEG).

8. The method of claim 1, wherein the somatic cell is an adult neural stem cell (NSC).

9-20. (canceled)

21. The method of claim 1, wherein the treatment with one or more agents comprises transfecting the cells with a vector comprising at least one gene.

22. (canceled)

23. The method of claim 21, wherein the genes are selected from the group consisting of: Jdhm2a, G9A and Nanog.

24. The method of claim 23, wherein Jdhm2a corresponds to the nucleotide sequence set forth in SEQ ID NO: 5 or SEQ ID NO: 7, G9A corresponds to the nucleotide sequence set forth in SEQ ID NO: 1 or SEQ ID NO: 3, and Nanog corresponds to the nucleotide sequence set forth in SEQ ID NO: 9 or SEQ ID NO: 11.

25-26. (canceled)

27. A reprogrammed cell produced by the method of claim 1.

28-29. (canceled)

30. A kit comprising a reprogrammed somatic cell produced according to the methods of claim 1, and instructions for use.

31. A method for reprogramming one or more somatic cells comprising:
contacting a somatic cell with an embryonic stem cell;
treating the cells with one or more agents that induces de-differentiation;
detecting the expression of one or more markers, where at least one marker indicates cell reprogramming;
selecting a cell that expresses the one or more markers;
thereby generating a reprogrammed cell.

32-55. (canceled)

56. A method of monitoring somatic cell fusion comprising:
contacting a somatic cell comprising a Cre recombinase protein with an embryonic cell, wherein the embryonic cell comprises a fluorescent Cre recombination excision reporter, and wherein detection of the fluorescent Cre recombination reporter is used to monitor cell fusion, or
A method of monitoring somatic cell fusion and reprogramming comprising:
contacting a somatic cell comprising an Oct4-GFP Cre recombinase protein with an embryonic cell, wherein the embryonic cell comprises a fluorescent Cre recombination excision reporter, and wherein detection of the fluorescent Cre recombination reporter is used to monitor cell fusion and detection of GFP is used to monitor reprogramming, or
A method of identifying an agent that alters somatic cell fusion comprising:
contacting a somatic cell comprising a Cre recombinase protein with an embryonic cell, wherein the embryonic cell comprises a fluorescent Cre recombination excision reporter, and wherein detection of the fluorescent Cre recombination reporter is used to monitor cell fusion;
contacting the cells with a candidate agent, wherein detection of the fluorescent Cre recombination reporter is used to identify an agent that alters somatic cell fusion, or
A method of identifying an agent that alters somatic cell fusion and reprogramming comprising:
contacting a somatic cell comprising a Oct4-GFP Cre recombinase protein with an embryonic cell, wherein the embryonic cell comprises a fluorescent Cre recombination excision reporter, and wherein detection of the fluorescent Cre recombination reporter is used to monitor cell fusion;
contacting the cells with a candidate agent, wherein detection of the fluorescent Cre recombination reporter is used to identify an agent that alters somatic cell fusion and detection of GFP is used to identify an agent that alters somatic cell reprogramming.

57. The method of claim 56, further comprising the step of monitoring somatic cell reprogramming, wherein the somatic cell comprises GFP and detection of GFP is used to monitor reprogramming.

58-89. (canceled)

90. A kit comprising a reprogrammed somatic cell produced according to the methods of claim 1, and instructions for use.

91. A kit for monitoring somatic cell fusion comprising a somatic cell comprising a Cre recombinase protein and an embryonic cell comprising a fluorescent Cre recombination excision reporter, and instructions for use according to the method of claim 56.

92. (canceled)


SEQUENCE LISTING (24 sequences)

SEQ ID NO: 1 - DNA, 3982 bp, Homo sapiens
gcaagcggcgatggcggcggcggcgggagctgcagcggcggcggccgccgagggggaggcccccgctgagatgggggcgctgctgctggagaaggaaaccagaggagccaccgagagagttcatggctctttgggggacacccctcgtagtgaagaaaccctgcccaaggccacccccgactccctggagcctgctggcccctcatctccagcctctgtcactgtcactgttggtgatgagggggctgacacccctgtaggggctacaccactcattggggatgaatctgagaatcttgagggagatggggacctccgtgggggccggatcctgctgggccatgccacaaagtcattcccctcttcccccagcaaggggggttcctgtcctagccgggccaagatgtcaatgacaggggcgggaaaatcacctccatctgtccagagtttggctatgaggctactgagtatgccaggagcccagggagctgcagcagcagggtctgaaccccctccagccaccacgagcccagagggacagcccaaggtccaccgagcccgcaaaaccatgtccaaaccaggaaatggacagcccccggtccctgagaagcggccccctgaaatacagcatttccgcatgagtgatgatgtccactcactgggaaaggtgacctcagatctggccaaaaggaggaagctgaactcaggaggtggcctgtcagaggagttaggttctgcccggcgttcaggagaagtgaccctgacgaaaggggaccccgggtccctggaggagtgggagacggtggtgggtgatgacttcagtctctactatgattcctactctgtggatgagcgcgtggactccgacagcaagtctgaagttgaagctctaactgaacaactaagtgaagaggaggaggaggaagaggaggaagaagaagaagaggaagaggaggaggaagaggaagaagaagaggaagatgaggagtcagggaatcagtcagataggagtggttccagtggccggcgcaaggccaagaagaaatggcgaaaagacagcccatgggtgaagccgtctcggaaacggcgcaagcgggagcctccgcgggccaaggagccacgaggagtgaatggtgtgggctcctcaggccccagtgagtacatggaggtccctctggggtccctggagctgcccagcgaggggaccctctcccccaaccacgctggggtgtccaatgacacatcttcgctggagacagagcgagggtttgaggagttgcccctgtgcagctgccgcatggaggcacccaagattgaccgcatcagcgagagggcggggcacaagtgcatggccactgagagtgtggacggagagctgtcaggctgcaatgccgccatcctcaagcgggagaccatgaggccatccagccgtgtggccctgatggtgctctgtgagacccaccgcgcccgcatggtcaaacaccactgctgcccgggctgcggctacttctgcacggcgggcaccttcctggagtgccaccctgacttccgtgtggcccaccgcttccacaaggcctgtgtgtctcagctgaatgggatggtcttctgtccccactgtggggaggatgcttctgaagctcaagaggtgaccatcccccggggtgacggggtgaccccaccggccggcactgcagctcctgcacccccacccctgtcccaggatgtccccgggagagcagacacttctcagcccagtgcccggatgcgagggcatggggaaccccggcgcccgccctgcgatcccctggctgacaccattgacagctcagggccctccctgaccctgcccaatgggggctgcctttcagccgtggggctgccactggggccaggccgggaggccctggaaaaggccctggtcatccaggagtcagagaggcggaagaagctccgtttccaccctcggcagttgtacctgtccgtgaagcagggcgagctgcagaaggtgatcctgatgctgttggacaacctggaccccaacttccagagcgaccagcagagcaagcgcacgcccctgcatgcagccgcccagaagggctccgtggagatctgccatgtgctgctgcaggctggagccaacataaatgcagtggacaaacagcagcggacgccactgatggaggccgtggtgaacaaccacctggaggtagcccgttacatggtgcagcgtggtggctgtgtctatagcaaggaggaggacggttccacctgcctccaccacgcagccaaaatcgggaacttggagatggtcagcctgctgctgagcacaggacaggtggacgtcaacgcccaggacagtggggggtggacgcccatcatctgggctgcagagcacaagcacatcgaggtgatccgcatgctactgacgcggggcgccgacgtcaccctcactgacaacgaggagaacatctgcctgcactgggcctccttcacgggcagcgccgccatcgccgaagtccttctgaatgcgcgctgtgacctccatgctgtcaactaccatggggacacccccctgcacatcgcagctcgggagagctaccatgactgcgtgctgttattcctgtcacgtggggccaaccctgagctgcggaacaaagagggggacacagcatgggacctgactcccgagcgctccgacgtgtggtttgcgcttcaactcaaccgcaagctccgacttggggtgggaaatcgggccatccgcacagagaagatcatctgccgggacgtggctcggggctatgagaacgtgcccattccctgtgtcaacggtgtggatggggagccctgccctgaggattacaagtacatctcagagaactgcgagacgtccaccatgaacatcgatcgcaacatcacccacctgcagcactgcacgtgtgtggacgactgctctagctccaactgcctgtgcggccagctcagcatccggtgctggtatgacaaggatgggcgattgctccaggaatttaacaagattgagcctccgctgattttcgagtgtaaccaggcgtgctcatgctggagaaactgcaagaaccgggtcgtacagagtggcatcaaggtgcggctacagctctaccgaacagccaagatgggctggggggtccgcgccctgcagaccatcccacaggggaccttcatctgcgagtatgtcggggagctgatctctgatgctgaggctgatgtgagagaggatgattcttacctcttcgacttagacaacaaggatggagaggtgtactgcatagatgcccgttactatggcaacatcagccgcttcatcaaccacctgtgtgaccccaacatcattcccgtccgggtcttcatgctgcaccaagacctgcgatttccacgcatcgccttcttcagttcccgagacatccggactggggaggagctagggtttgactatggcgaccgcttctgggacatcaaaagcaaatatttcacctgccaatgtggctctgagaagtgcaagcactcagccgaagccattgccctggagcagagccgtctggcccgcctggacccacaccctgagctgctgcccgagctcggctccctgccccctgtcaacacatgagaacggaccacaccctctctccccagcatggatggccacagctcagccgcctcctctgccaccagctgctcgcagcccatgcctgggggtgctgccatcttctctccccaccaccctttcacacattcctgaccagagatcccagccaggccctggaggtctgacagcccctccctcccagagctggttcctccctgggagggcaacttcagggctggccaccccccgtgttccccatcctcagttgaagtttgatgaattgaagtcgggcctctatgccaactggttccttttgttctcaataaatgttgggtttggtaataaaaaaaaaaaaaaaaa

SEQ ID NO: 2 - protein, 1210 aa, Homo sapiens
MAAAAGAAAAAAAEGEAPAEMGALLLEKETRGATERVHGSLGDTPRSEETLPKATPDSLEPAGPSSPASVTVTVGDEGADTPVGATPLIGDESENLEGDGDLRGGRILLGHATKSFPSSPSKGGSCPSRAKMSMTGAGKSPPSVQSLAMRLLSMPGAQGAAAAGSEPPPATTSPEGQPKVHRARKTMSKPGNGQPPVPEKRPPEIQHFRMSDDVHSLGKVTSDLAKRRKLNSGGGLSEELGSARRSGEVTLTKGDPGSLEEWETVVGDDFSLYYDSYSVDERVDSDSKSEVEALTEQLSEEEEEEEEEEEEEEEEEEEEEEEEDEESGNQSDRSGSSGRRKAKKKWRKDSPWVKPSRKRRKREPPRAKEPRGVNGVGSSGPSEYMEVPLGSLELPSEGTLSPNHAGVSNDTSSLETERGFEELPLCSCRMEAPKIDRISERAGHKCMATESVDGELSGCNAAILKRETMRPSSRVALMVLCETHRARMVKHHCCPGCGYFCTAGTFLECHPDFRVAHRFHKACVSQLNGMVFCPHCGEDASEAQEVTIPRGDGVTPPAGTAAPAPPPLSQDVPGRADTSQPSARMRGHGEPRRPPCDPLADTIDSSGPSLTLPNGGCLSAVGLPLGPGREALEKALVIQESERRKKLRFHPRQLYLSVKQGELQKVILMLLDNLDPNFQSDQQSKRTPLHAAAQKGSVEICHVLLQAGANINAVDKQQRTPLMEAVVNNHLEVARYMVQRGGCVYSKEEDGSTCLHHAAKIGNLEMVSLLLSTGQVDVNAQDSGGWTPIIWAAEHKHIEVIRMLLTRGADVTLTDNEENICLHWASFTGSAAIAEVLLNARCDLHAVNYHGDTPLHIAARESYHDCVLLFLSRGANPELRNKEGDTAWDLTPERSDVWFALQLNRKLRLGVGNRAIRTEKIICRDVARGYENVPIPCVNGVDGEPCPEDYKYISENCETSTMNIDRNITHLQHCTCVDDCSSSNCLCGQLSIRCWYDKDGRLLQEFNKIEPPLIFECNQACSCWRNCKNRVVQSGIKVRLQLYRTAKMGWGVRALQTIPQGTFICEYVGELISDAEADVREDDSYLFDLDNKDGEVYCIDARYYGNISRFINHLCDPNIIPVRVFMLHQDLRFPRIAFFSSRDIRTGEELGFDYGDRFWDIKSKYFTCQCGSEKCKHSAEAIALEQSRLARLDPHPELLPELGSLPPVNT

SEQ ID NO: 3 - DNA, 4026 bp, Mus musculus
atgcggggtctgccgagagggagggggctgatgcgggcccgggggcgggggcgtgcggcccccacgggcggccgcggccgcggtcgggggggcgcccaccgagggcgaggtaggccccgaagcctgctctcgctgcccagggcccaggcgtcttgggccccccagctgcctgccgggctgaccggccccccggttccttgtctcccctcccagggggaggcccccgctgagatgggggcgctgctgctggagaaggagccccgaggagccgccgagagagttcatagctctttgggggacacccctcagagtgaggagacccttcccaaggccaaccccgactccttggagcctgccggcccctcctctccggcctctgtcactgtcaccgtcggcgatgagggggctgacacccctgtcggggccgcatcactcatcggggacgaacccgagagcctggagggagatgggggtcgcatcgtgctgggccatgccacaaagtcgttcccctcttcccccagcaaggggggtgcctgtcccagtcgggccaaaatgtcaatgacaggggcaggaaagtcgcccccctcggtccagagtttggccatgaggctgttgagcatgcccggggcccagggagctgcaactgctgggcctgaaccctctccggcaacaactgccgcccaggaggggcagcccaaagtgcaccgagcccggaaaaccatgtccaaacctagcaacggacagcctccaatccctgagaagcggccccctgaagtccagcatttccgcatgagtgatgacatgcatctggggaaggtgacttcagatgtggccaaaaggaggaagctgaactctggtagcctgtccgaggacttgggctctgccgggggctcaggagatataatcctggagaagggagagcccaggcccctggaggagtgggagacggtggtgggcgatgacttcagcctgtactatgatgcgtactctgtggatgagcgggtggactctgacagcaagtctgaagtcgaagctctagctgaacagttgagtgaggaggaggaggaggaagaggaggaagaagaagaagaggaggaggaggaggaagaggaggaggaggaagaagaggacgaggagtcgggcaatcagtcagacaggagcggttctagtggccggcgcaaggccaagaagaaatggcggaaagacagcccgtgggtgaagccatctagaaaacggcggaaacgagagcctccgagggccaaggagccaagaggagtgaatggtgtgggttcctcagggcccagtgagtacatggaggttcctctggggtccctggagctgcccagcgaggggaccctctcccccaaccacgctggggtctccaatgacacgtcttcactggagacagaacgcgggtttgaggagctgcccctctgcagctgccgcatggaggctcccaagattgaccgcatcagcgagagagcagggcacaagtgcatggccacagagagtgtggatggagagctcctgggctgcaatgctgccatccttaagcgggagaccatgcggccgtctagccgcgtggcgctgatggtgctctgtgaggcccatcgagcccgcatggtcaagcaccattgctgcccgggctgcggctacttctgcacagcgggcaccttcctggaatgccaccccgactttcgtgtagctcaccgcttccataaggcctgcgtatcccagctcaatgggatggtcttctgtccccactgtggagaggatgcctcagaggcccaggaggtgaccattcctcggggcgatgggggaacacccccaattggcaccgcagctcctgctctgccacccctggcacatgatgccccagggcgagcggatacctcccagcctagcgcccgaatgcgagggcatggagagccgcggcgcccgccctgtgatcccctggctgacaccatcgacagctcagggccttcactgactctgcctaatgggggctgcctctccgctgtgggtctgcccccagggccgggcagggaagccctggaaaaagccttggtcatccaggagtctgagaggcggaagaagctgcgattccacccacggcagctgtacctgtcggtgaagcagggggagctgcagaaggtgatccttatgctgttagacaacctggaccccaacttccagagcgaccagcagagcaagcgcacgcccctgcacgcggccgcccagaaggggtcggtagagatctgtcatgtgctgctgcaggcaggagccaacatcaatgccgtagataagcaacaacgcacgccactaatggaggccgtggtgaacaaccacctggaggtggcacgctacatggtgcagttaggtggctgtgtctacagcaaggaagaggatggctccacctgtctacatcatgcagccaaaattgggaacttggaaatggtcagcctgctactgagcacaggacaggtggacgtcaatgcccaggacagtgggggctggacgcccatcatctgggcagccgagcacaagcacatcgatgtgattcgtatgctgctgacccggggtgccgatgtcaccctgactgacaatgaggaaaacatctgcctgcactgggcctccttcacgggtagtgccgccatcgctgaggtccttctgaatgcccagtgtgatctccatgctgtcaactaccatggggacacgcccctgcacatagccgccagggagagctaccatgactgtgttctgttgttcctgtctcgtggagccaaccctgagcttcggaacaaagaaggagacacggcatgggatctgaccccagagcgctctgatgtgtggtttgcactgcagctcaatcgaaagcttaggcttggggtagggaaccgggctgtccgcaccgagaagatcatctgccgggacgtagcccgaggctatgagaatgtacccatcccctgtgtcaatggtgtggatggggagccgtgcccggaggactacaagtacatctctgagaactgcgagacatcgaccatgaacatcgaccgcaacatcacccatctgcagcactgcacgtgtgtggatgactgctccagctccaattgcctatgtggtcagctcagtatccgatgctggtatgacaaggacgggcggctgctccaggagtttaacaagatcgagccccccctgatctttgagtgtaaccaggcatgctcctgctggagaagctgcaagaaccgcgtggtgcagagcggcatcaaggtacggctgcagctctaccggactgccaagatgggctggggggtccgagccttgcagaccatcccccagggcacgttcatctgcgagtatgtaggagagctgatctctgatgccgaggctgatgtgagagaggatgattcttacctcttcgatttagataacaaggatggcgaggtttactgcattgatgcccgttactatggcaacatcagccgattcattaaccacctgtgtgaccccaacatcatccctgtccgggttttcatgctgcaccaagatctacggttcccacgcattgccttcttcagctccagggacatccggactggggaggagctgggctttgactacggtgaccgattctgggacatcaagagcaagtatttcacctgccagtgtggctctgagaagtgcaagcattcagcggaggccatcgccctggagcagagccgcctggcccggctggacccccacccggagctgctccctgacctcagctccctgccccccatcaacacctgaggactcttaaaatccaggccgggcactgcccttcagacatttctccatcagagaccccagtaaggcctggaaggtcgatggcccctctcccagagctggtttctcactgggagtgcaagtgacttcagggctggccttccccactgagcctggcctcagttagctgattgaagttgggcctctgccagctgattttctgtgttctcaataaatgttgggtttggtaaaaaaaaa

SEQ ID NO: 4 - protein, 1263 aa, Mus musculus
MRGLPRGRGLMRARGRGRAAPTGGRGRGRGGAHRGRGRPRSLLSLPRAQASWAPQLPAGLTGPPVPCLPSQGEAPAEMGALLLEKEPRGAAERVHSSLGDTPQSEETLPKANPDSLEPAGPSSPASVTVTVGDEGADTPVGAASLIGDEPESLEGDGGRIVLGHATKSFPSSPSKGGACPSRAKMSMTGAGKSPPSVQSLAMRLLSMPGAQGAATAGPEPSPATTAAQEGQPKVHRARKTMSKPSNGQPPIPEKRPPEVQHFRMSDDMHLGKVTSDVAKRRKLNSGSLSEDLGSAGGSGDIILEKGEPRPLEEWETVVGDDFSLYYDAYSVDERVDSDSKSEVEALAEQLSEEEEEEEEEEEEEEEEEEEEEEEEEDEESGNQSDRSGSSGRRKAKKKWRKDSPWVKPSRKRRKREPPRAKEPRGVNGVGSSGPSEYMEVPLGSLELPSEGTLSPNHAGVSNDTSSLETERGFEELPLCSCRMEAPKIDRISERAGHKCMATESVDGELLGCNAAILKRETMRPSSRVALMVLCEAHRARMVKHHCCPGCGYFCTAGTFLECHPDFRVAHRFHKACVSQLNGMVFCPHCGEDASEAQEVTIPRGDGGTPPIGTAAPALPPLAHDAPGRADTSQPSARMRGHGEPRRPPCDPLADTIDSSGPSLTLPNGGCLSAVGLPPGPGREALEKALVIQESERRKKLRFHPRQLYLSVKQGELQKVILMLLDNLDPNFQSDQQSKRTPLHAAAQKGSVEICHVLLQAGANINAVDKQQRTPLMEAVVNNHLEVARYMVQLGGCVYSKEEDGSTCLHHAAKIGNLEMVSLLLSTGQVDVNAQDSGGWTPIIWAAEHKHIDVIRMLLTRGADVTLTDNEENICLHWASFTGSAAIAEVLLNAQCDLHAVNYHGDTPLHIAARESYHDCVLLFLSRGANPELRNKEGDTAWDLTPERSDVWFALQLNRKLRLGVGNRAVRTEKIICRDVARGYENVPIPCVNGVDGEPCPEDYKYISENCETSTMNIDRNITHLQHCTCVDDCSSSNCLCGQLSIRCWYDKDGRLLQEFNKIEPPLIFECNQACSCWRSCKNRVVQSGIKVRLQLYRTAKMGWGVRALQTIPQGTFICEYVGELISDAEADVREDDSYLFDLDNKDGEVYCIDARYYGNISRFINHLCDPNIIPVRVFMLHQDLRFPRIAFFSSRDIRTGEELGFDYGDRFWDIKSKYFTCQCGSEKCKHSAEAIALEQSRLARLDPHPELLPDLSSLPPINT

SEQ ID NO: 5 - DNA, 4907 bp, Homo sapiens
taatgggggtcgcccgggagtcggaagggggaggggaaagggaggaggcagccaaggaattgtttttttctctggccccgccctcgcccggggggccaatggtgatgatctgtttcccccggagcctcgcccagctcctgtgtttcagccaatgagcggcggaagcggctccgaggggggcgggtccgggaggctgtgcgtgtcttgtgagagctcttgaaccaagtcagcgctggagtcggctaggcggctggaaacggcggctgccgccggtgactcagggaggcgggaggcgggggaggagctcttcctgcaggcgtggaaaccatggtgctcacgctcggagaaagttggccggtattggtggggaggaggtttctcagtctgtccgcagccgacggcagcgatggcagccacgacagctgggacgtggagcgcgtcgccgagtggccctggctctccgggaccattcgagctgtttcccacaccgacgttaccaagaaggatctgaaggtgtgtgtggaatttgatggggaatcttggaggaaaagaagatggatagaagtctacagccttctaaggagagcatttttagtagaacataatttggttttagctgaacgaaagtcacctgaaatttctgaacgaattgtacagtggcctgcaataacgtacaaacctctgttggacaaagctggtttgggatccataacttctgttcgctttctgggagatcaacaaagagtatttctttctaaagaccttttgaagcctatacaggatgtaaacagtcttcgactttctcttacggataatcagattgtcagtaaagaatttcaagctttgattgtgaagcatttagatgaaagccatcttttaaaaggtgacaaaaacttagttggttcagaagtaaaaatttatagcttggacccatctactcagtggttttcagcaaccgttataaatggaaacccagcatcaaaaactcttcaagtcaactgtgaggagattccagcactgaaaattgttgatccgtcactgattcatgttgaagttgtacacgataaccttgtgacatgtggtaattctgcaagaattggagctgtaaaacgcaagtcttctgagaataatggaaccctggtttccaaacaagcaaaatcttgctctgaggcctctcccagtatgtgtcctgtgcagtctgtacctacaacagtttttaaggagatactgcttggctgtactgcggcaactccacctagtaaggacccaagacagcaaagtactccccaggctgccaactctccacctaaccttggagcaaaaattcctcaaggatgtcataaacaaagtttaccagaggaaatttcttcctgtctaaatacaaagtctgaagctctgagaacaaaaccagatgtctgcaaagcagggttgctctcaaagtcctctcagattggaactggagacttgaaaattctgactgagccaaaaggcagctgtactcagcctaagacaaacactgatcaggaaaacagattggagtctgttccacaagcattgactggccttcctaaggagtgcttacctacaaaggcttcttctaaggcagaattggaaattgccaatcctcctgaactgcagaagcacctagaacatgcaccttccccatcggatgtttcaaatgcaccagaagtgaaagcaggtgtcaatagtgatagccctaataactgttcaggaaaaaaggtagaaccttcagctttagcttgccgatcacagaatttaaaggaatcttcagtaaaagtagataatgaaagctgttgttcaagaagcaacaataaaatccagaatgccccatccaggaagtcggttttgacagacccagctaaactcaaaaagctgcaacagagtggcgaggccttcgtacaggatgattcttgtgtgaacatcgtggcacagttgcctaaatgccgagagtgtcgcttggacagtctccgcaaggataaggagcaacagaaggactcacctgtgttttgccgcttctttcacttcaggaggttacaattcaacaaacatggtgtgttgcgggtagaaggcttcttaacaccaaacaagtatgacaatgaagcaattggcttgtggttacctttaaccaaaaacgttgtggggattgatttggacacagcaaagtacatcttggccaacattggagaccacttctgtcaaatggtgatttctgaaaaggaagctatgtcaactattgagccacacagacaggttgcttggaagcgagctgtcaaaggtgttcgagaaatgtgtgatgtgtgcgacaccaccatcttcaacctgcactgggtgtgtcctcggtgtgggtttggagtatgtgtggactgctaccggatgaagagaaagaattgccaacagggtgctgcttacaagactttctcttggctaaaatgtgtgaagagtcagatacatgaaccagagaacttaatgcccacacagatcattcctggaaaagcactctatgatgttggagacattgttcattctgtaagagcgaaatggggaataaaggcaaactgcccttgttcaaacaggcaattcaaactcttttcaaagccagcctcaaaggaagacctaaaacagacttctttagctggagaaaaaccgactcttggtgcagtgctccagcagaatccctcagtgttggagccagcagctgtgggtggggaagcagcctccaagccagccggcagcatgaagcctgcctgtccagccagcacatctcctctaaactggctggccgacctaaccagcgggaatgtcaacaaggaaaacaaggaaaaacaaccaacaatgccaattttaaagaatgaaatcaaatgccttccacccctcccacctttaagcaaatccagcacagtcctccatacgtttaacagcacaattttgacacccgtaagcaacaacaattctggtttcctccggaatctcttgaattcttctacaggaaagacagaaaatggactcaagaatacaccaaaaatccttgatgacatctttgcctctttggtgcaaaataagacgacttctgatttatctaagaggcctcaaggactaaccatcaagcccagcattctgggctttgacactcctcactattggctttgtgataatcgcttgctgtgcttgcaagaccccaacaataagagcaactggaatgtgtttagggagtgctggaaacaagggcagccagtgatggtgtctggagtgcatcataaattgaactctgaactttggaaacctgaatccttcaggaaagagtttggtgagcaggaagtagacctagttaattgtaggaccaatgaaatcatcacaggagccacagtaggagacttctgggatggatttgaagatgttccaaatcgtttgaaaaatgaaaaagaaccaatggtgttgaaacttaaggactggccaccaggagaagattttagagatatgatgccttccaggtttgatgatctgatggccaacattccactgcccgagtacacaaggcgagatggcaaactgaatttggcctctaggctgccaaactactttgttcggccagatctgggccccaagatgtataatgcttatggattaatcactcctgaagatcggaaatatggaacaacaaatcttcacttagatgtatctgatgcagctaatgtcatggtctatgtgggaattcccaaaggacagtgtgagcaagaagaagaagtccttaagaccatccaagatggagattctgacgaactcacaataaagcgatttattgaaggaaaagagaagccaggagcactgtggcacatatatgctgcaaaggacacggagaagataagggaatttcttaaaaaggtatcagaagagcaaggtcaagaaaacccagcagaccacgatcctattcatgatcaaagctggtatttagaccgatcattaagaaaacgtcttcatcaagagtatggagttcaaggctgggctattgtacagtttcttggggatgtggtgtttatcccggcaggagctccacatcaggttcataacttatatagctgcatcaaagtggctgaagattttgtttctccagagcatgttaaacactgcttctggcttactcaggaattccgatatctgtcacagactcataccaatcacgaagataaattacaggtgaagaatgttatctaccatgcagtgaaagatgcagttgctatgctgaaagccagtgaatccagttttggcaaaccttaatctccctgcacattggaaatgaattacaggcagctgttcaaactcttcaggcaggattcctgtggactttgagattcatgttacctcatcttcttttttaaactgtacccaacttgtgagggtactctgtctaatgtatatttctagtgtttacagacagtaaatgtgtatatgtagtaactatttacagaacatgcatccttaaactgtgacttctcacctagtgcagaacttttaccaggctgtaaaagcaaaacctcgtatcagctctggaacaatacctgcagttattcttcagctgtttggacaacttagattgggtttataactattaggaatcactgcacagtttatttgggttgtgttttgtgtctgagtcccctccctcatcccttagggtccagaagagcaatggaggaagtgacagctaatgttgcagttcttattgtatggcataggactggcattatatagcagaaatcaactactgtacaatttcttggggttaaccatctttagttaaatggaattttaatttaaatgacgctttgctaattttaagtgttaagcattttgcattaaaatattcatataataaaaaaaaaaaaaaaaaa

SEQ ID NO: 6 - protein, 1321 aa, Homo sapiens
MVLTLGESWPVLVGRRFLSLSAADGSDGSHDSWDVERVAEWPWLSGTIRAVSHTDVTKKDLKVCVEFDGESWRKRRWIEVYSLLRRAFLVEHNLVLAERKSPEISERIVQWPAITYKPLLDKAGLGSITSVRFLGDQQRVFLSKDLLKPIQDVNSLRLSLTDNQIVSKEFQALIVKHLDESHLLKGDKNLVGSEVKIYSLDPSTQWFSATVINGNPASKTLQVNCEEIPALKIVDPSLIHVEVVHDNLVTCGNSARIGAVKRKSSENNGTLVSKQAKSCSEASPSMCPVQSVPTTVFKEILLGCTAATPPSKDPRQQSTPQAANSPPNLGAKIPQGCHKQSLPEEISSCLNTKSEALRTKPDVCKAGLLSKSSQIGTGDLKILTEPKGSCTQPKTNTDQENRLESVPQALTGLPKECLPTKASSKAELEIANPPELQKHLEHAPSPSDVSNAPEVKAGVNSDSPNNCSGKKVEPSALACRSQNLKESSVKVDNESCCSRSNNKIQNAPSRKSVLTDPAKLKKLQQSGEAFVQDDSCVNIVAQLPKCRECRLDSLRKDKEQQKDSPVFCRFFHFRRLQFNKHGVLRVEGFLTPNKYDNEAIGLWLPLTKNVVGIDLDTAKYILANIGDHFCQMVISEKEAMSTIEPHRQVAWKRAVKGVREMCDVCDTTIFNLHWVCPRCGFGVCVDCYRMKRKNCQQGAAYKTFSWLKCVKSQIHEPENLMPTQIIPGKALYDVGDIVHSVRAKWGIKANCPCSNRQFKLFSKPASKEDLKQTSLAGEKPTLGAVLQQNPSVLEPAAVGGEAASKPAGSMKPACPASTSPLNWLADLTSGNVNKENKEKQPTMPILKNEIKCLPPLPPLSKSSTVLHTFNSTILTPVSNNNSGFLRNLLNSSTGKTENGLKNTPKILDDIFASLVQNKTTSDLSKRPQGLTIKPSILGFDTPHYWLCDNRLLCLQDPNNKSNWNVFRECWKQGQPVMVSGVHHKLNSELWKPESFRKEFGEQEVDLVNCRTNEIITGATVGDFWDGFEDVPNRLKNEKEPMVLKLKDWPPGEDFRDMMPSRFDDLMANIPLPEYTRRDGKLNLASRLPNYFVRPDLGPKMYNAYGLITPEDRKYGTTNLHLDVSDAANVMVYVGIPKGQCEQEEEVLKTIQDGDSDELTIKRFIEGKEKPGALWHIYAAKDTEKIREFLKKVSEEQGQENPADHDPIHDQSWYLDRSLRKRLHQEYGVQGWAIVQFLGDVVFIPAGAPHQVHNLYSCIKVAEDFVSPEHVKHCFWLTQEFRYLSQTHTNHEDKLQVKNVIYHAVKDAVAMLKASESSFGKP

SEQ ID NO: 7 - DNA, 4868 bp, Mus musculus
aagtgtcgagtcgcgagcgagtccacggcggctccgaggccgctcggggcggggatcggtcgctgagacgggccctaggcactaagagggagccttttctttaacagggcgaggggacgaacacttaggcaaaagcactggcgccgcggctcagtcctcccttctctctctcagtgtccagctttgaaagggaggagcccttcctgctggcgtggaaaccatggtgctcacgctcggagaaagttggccagtattggtggggaagcgattcctcagtctgtccgcagccgaaggcaacgaaggcggccaggacaactgggacttggagcgcgttgccgagtggccctggctgtcggggaccattcgagctgtttcccacaccgacgttactaagaaagacttgaaggtgtgtgtggagtttgatggggagtcttggagaaagagaagatggatagatgtctacagccttcagagaaaagcatttttagtagagcataacctggttttggcagaacgaaaatcacctgaagttcctgagcaagttattcagtggcctgcaataatgtacaaatctcttctagacaaagctggcttgggagccataacttctgttcggtttcttggagatcaacaaagtgtatttgtttccaaagaccttttgaaacctatacaggatgttaacagtcttcggctttcccttactgataatcagacagtcagtaaggaatttcaagctttgattgtaaaacatttggatgaaagccatcttttacaaggtgacaagaaccttgttggttcagaagtaaaaatttatagcttggacccatctactcagtggttttcagcaactgttgtacatggaaacccatcatccaaaactcttcaagtcaactgtgaggagattccagcactgaaaattgtcgacccagcactgattcatgttgaagttgtacatgacaactttgtgacatgtggtaattctacaagaactggagctgtaaaacgcaagtcttctgagaataacggaagttcggtttctaaacaagcaaaatcttgttctgaggcctctcccagtatgtgtcctgtacagtctgttcccacaacagtgtttaaggagatcctgcttggctgtactgcagcaactccatctagcaaggacccaagacagcaaaatactccccaggcagccaattctccacctaacattggagcaaaacttcctcaaggatgtcataagcagaacttaccagaagaactttcttcctgtctaaacacaaaacctgaagtaccgagaacaaaaccagatgtctgcaaagaaggattactttcttcaaaatcttctcaggttggagctggagacttgaaaattctgagtgagcccaaaggtagctgtatccagcctaaaacaaacactgatcaggagagcagactggagtctgctccacagccagtcactggccttccaaaggagtgcttgcctgcaaagacttcctctaaggcagaactggacattgccaccactcctgaactgcagaagcatctagaacatgcagcttccacatccgatgacctttcagataagccagaagtgaaagcaggtgtcactagccttaatagttgtgcagaaaagaaggtcgaaccttcacatttaggttcccagtcacagaatttaaaggaaacttcagtaaaagtagataatgaaagctgttgtacaagaagcagtaataaaacccagactcccccagcccggaagtcagttttgacagacccagataaagtcaggaagctgcagcagagcggagaggcctttgttcaggatgactcctgtgttaacatcgtggcacagctgcccaagtgtcgggagtgtcgactagacagcctgcgcaaggataaggaccagcagaaggactctcctgtgttttgtcgctttttccacttcaggagattacaattcaacaagcatggtgtgttgcgggtagaaggcttcttaacaccaaacaagtatgacagtgaagcgattggcttgtggctgcctttgaccaaaaatgttgtggggactgatttggacacagcaaaatatatcctggccaatattggagaccacttctgtcaaatggtgatttctgagaaggaagctatgtcgactattgagccacacagacaggttgcttggaaacgagctgtcaaaggagttagagaaatgtgtgatgtgtgtgacacaaccattttcaacctgcactgggtgtgccctcggtgtgggtttggagtatgtgtagattgctaccggatgaagaggaagaactgccaacagggtgctgcctacaagactttctcttggataaggtgtgtgaagagtcagatacatgagcctgagaacctgatgcccacacagattattcctggcaaagccctctacgatgttggagacattgtgcattctgtcagagcaaaatggggcataaaggccaattgtccctgctccaacaggcagttcaagctcttctcaaagccagccttaaaggaagacctgaaacagacatccttgtctggagaaaaaccaactcttgggaccatggtccagcaaagttcccctgttttggagccagtggctgtgtgcggggaagcagcctccaagccagccagcagcgtgaagcccacctgtcccaccagcacttcacctttaaactggctagctgaccttaccagtgggaatgtcaacaaggagaataaggaaaaacagctgactatgccaattttaaagaatgaaatcaaatgccttccacccttgccccctctgaacaagcccagcacagtcctccatacttttaacagcaccatcttgacacctgtgagcaacaataattcaggtttccttagaaatcttttgaattcatccacagcaaagacagaaaatggattgaaaaacacacccaaaattcttgatgacatctttgcctctttggtgcaaaacaagacttcttctgattcatccaagaggcctcaaggactgacaatcaagcctagcattcttggctttgacactcctcactactggctgtgtgacaaccgcctgctgtgcttgcaagaccccaacaataagagcaattggaatgtttttagggaatgctggaaacaagggcagccagtgatggtgtcgggcgtgcatcataaattaaacactgaactctggaaacccgagtccttcagaaaagagtttggtgagcaggaagtagacctagtcaattgtaggaccaatgaaatcatcactggagccacagtcggagacttctgggatggatttgaagatgttccaaaccgtttgaaaaacgacaaagaaaaagaaccaatggtgttgaaacttaaggactggccgccaggagaagacttcagagacatgatgccttccaggtttgatgatctgatggccaacattccactgcctgagtacaccaggcgagatggcaaactgaacctggcttccagactgccaaactactttgtacggccagacctgggccccaagatgtacaatgcttatggattgatcactccagaggatcggaaatatgggaccacaaatcttcacttagatgtatctgatgcagccaatgtcatggtttatgtgggaattcccaaaggacagtgtgaacaagaagaagaagtccttagaaccatccaagatggagattctgatgaactcacaatcaagagatttattgaaggaaaagagaagccaggagccctttggcacatatatgctgctaaagacacagagaagataagagaattccttaaaaaggtatcagaggagcagggtcaagacaaccctgcagaccatgaccctatccacgatcagagctggtatttagaccgatcgctgagaaagcgcctctatcaagagtacggcgtgcaaggctgggctattgtacagtttcttggggatgtggtgtttatcccagcaggagcgccacatcaggttcataacttatacagctgtatcaaagtggctgaagactttgtgtctccagagcatgttaaacactgcttctggcttactcaggaattccgttacttgtcacagactcataccaaccatgaagataaattgcaggtgaaaaatgttatctaccatgcagtgaaagatgcagttgctatgctgaaagccagtgaatccagtttgggcaaaccttaactcttctctgcacaatggagatgaattattggcagctgatcaaactcttcaggcaggattcctgtggactttgagatttcctgttacctcatcttcttttttaaagtacacctgacttgggagggtactgtctctaatgtatatttctagtgtttacagacactaagtgtgtatatgtagtaactatttacagaccacgcatccttatactgtgacttcacctagatcttctaccaagctgaagaccctgctggctctgaaacaatccttgcagttactccccagctgttcgtctggacagctcattcaagtggatttttaactattagggatcactgcgaagtttcgttggattttattttatgtccttcagagcaccctcccccacccactagggtccagaagagcaatggaggaagtgacagctaatggtgcagttctaaatatatattgcataggactggcattatatagcagaaataactactgtataattcttggggttaaccatctttagttaatggaattttaatttaaatgaagctttgctaattttaagtggtaagcattttgcattaaaatattcctataatattttgtcgctgttctttgtcctttattctttgtttactctctcgaaaataaaagggctaaactattgaaaaaaaaaaaaaaa

SEQ ID NO: 8 - protein, 1323 aa, Mus musculus
MVLTLGESWPVLVGKRFLSLSAAEGNEGGQDNWDLERVAEWPWLSGTIRAVSHTDVTKKDLKVCVEFDGESWRKRRWIDVYSLQRKAFLVEHNLVLAERKSPEVPEQVIQWPAIMYKSLLDKAGLGAITSVRFLGDQQSVFVSKDLLKPIQDVNSLRLSLTDNQTVSKEFQALIVKHLDESHLLQGDKNLVGSEVKIYSLDPSTQWFSATVVHGNPSSKTLQVNCEEIPALKIVDPALIHVEVVHDNFVTCGNSTRTGAVKRKSSENNGSSVSKQAKSCSEASPSMCPVQSVPTTVFKEILLGCTAATPSSKDPRQQNTPQAANSPPNIGAKLPQGCHKQNLPEELSSCLNTKPEVPRTKPDVCKEGLLSSKSSQVGAGDLKILSEPKGSCIQPKTNTDQESRLESAPQPVTGLPKECLPAKTSSKAELDIATTPELQKHLEHAASTSDDLSDKPEVKAGVTSLNSCAEKKVEPSHLGSQSQNLKETSVKVDNESCCTRSSNKTQTPPARKSVLTDPDKVRKLQQSGEAFVQDDSCVNIVAQLPKCRECRLDSLRKDKDQQKDSPVFCRFFHFRRLQFNKHGVLRVEGFLTPNKYDSEAIGLWLPLTKNVVGTDLDTAKYILANIGDHFCQMVISEKEAMSTIEPHRQVAWKRAVKGVREMCDVCDTTIFNLHWVCPRCGFGVCVDCYRMKRKNCQQGAAYKTFSWIRCVKSQIHEPENLMPTQIIPGKALYDVGDIVHSVRAKWGIKANCPCSNRQFKLFSKPALKEDLKQTSLSGEKPTLGTMVQQSSPVLEPVAVCGEAASKPASSVKPTCPTSTSPLNWLADLTSGNVNKENKEKQLTMPILKNEIKCLPPLPPLNKPSTVLHTFNSTILTPVSNNNSGFLRNLLNSSTAKTENGLKNTPKILDDIFASLVQNKTSSDSSKRPQGLTIKPSILGFDTPHYWLCDNRLLCLQDPNNKSNWNVFRECWKQGQPVMVSGVHHKLNTELWKPESFRKEFGEQEVDLVNCRTNEIITGATVGDFWDGFEDVPNRLKNDKEKEPMVLKLKDWPPGEDFRDMMPSRFDDLMANIPLPEYTRRDGKLNLASRLPNYFVRPDLGPKMYNAYGLITPEDRKYGTTNLHLDVSDAANVMVYVGIPKGQCEQEEEVLRTIQDGDSDELTIKRFIEGKEKPGALWHIYAAKDTEKIREFLKKVSEEQGQDNPADHDPIHDQSWYLDRSLRKRLYQEYGVQGWAIVQFLGDVVFIPAGAPHQVHNLYSCIKVAEDFVSPEHVKHCFWLTQEFRYLSQTHTNHEDKLQVKNVIYHAVKDAVAMLKASESSLGKP

SEQ ID NO: 9 - DNA, 2098 bp, Homo sapiens
attataaatctagagactccaggattttaacgttctgctggactgagctggttgcctcatgttattatgcaggcaactcactttatcccaatttcttgatacttttccttctggaggtcctatttctctaacatcttccagaaaagtcttaaagctgccttaaccttttttccagtccacctcttaaattttttcctcctcttcctctatactaacatgagtgtggatccagcttgtccccaaagcttgccttgctttgaagcatccgactgtaaagaatcttcacctatgcctgtgatttgtgggcctgaagaaaactatccatccttgcaaatgtcttctgctgagatgcctcacacggagactgtctctcctcttccttcctccatggatctgcttattcaggacagccctgattcttccaccagtcccaaaggcaaacaacccacttctgcagagaagagtgtcgcaaaaaaggaagacaaggtcccggtcaagaaacagaagaccagaactgtgttctcttccacccagctgtgtgtactcaatgatagatttcagagacagaaatacctcagcctccagcagatgcaagaactctccaacatcctgaacctcagctacaaacaggtgaagacctggttccagaaccagagaatgaaatctaagaggtggcagaaaaacaactggccgaagaatagcaatggtgtgacgcagaaggcctcagcacctacctaccccagcctttactcttcctaccaccagggatgcctggtgaacccgactgggaaccttccaatgtggagcaaccagacctggaacaattcaacctggagcaaccagacccagaacatccagtcctggagcaaccactcctggaacactcagacctggtgcacccaatcctggaacaatcaggcctggaacagtcccttctataactgtggagaggaatctctgcagtcctgcatgcagttccagccaaattctcctgccagtgacttggaggctgccttggaagctgctggggaaggccttaatgtaatacagcagaccactaggtattttagtactccacaaaccatggatttattcctaaactactccatgaacatgcaacctgaagacgtgtgaagatgagtgaaactgatattactcaatttcagtctggacactggctgaatccttcctctcccctcctcccatccctcataggatttttcttgtttggaaaccacgtgttctggtttccatgatgcccatccagtcaatctcatggagggtggagtatggttggagcctaatcagcgaggtttctttttttttttttttcctattggatcttcctggagaaaatactttttttttttttttttttgaaacggagtcttgctctgtcgcccaggctggagtgcagtggcgcggtcttggctcactgcaagctccgtctcccgggttcacgccattctcctgcctcagcctcccgagcagctgggactacaggcgcccgccacctcgcccggctaatattttgtatttttagtagagacggggtttcactgtgttagccaggatggtctcgatctcctgaccttgtgatccacccgcctcggcctccctaacagctgggatttacaggcgtgagccaccgcgccctgcctagaaaagacattttaataaccttggctgccgtctctggctatagataagtagatctaatactagtttggatatctttagggtttagaatctaacctcaagaataagaaatacaagtacaaattggtgatgaagatgtattcgtattgtttgggattgggaggctttgcttattttttaaaaactattgaggtaaagggttaagctgtaacatacttaattgatttcttaccgtttttggctctgttttgctatatcccctaatttgttggttgtgctaatctttgtagaaagaggtctcgtatttgctgcatcgtaatgacatgagtactgctttagttggtttaagttcaaatgaatgaaacaactatttttcctttagttgattttaccctgatttcaccgagtgtttcaatgagtaaatatacagcttaaacat

SEQ ID NO: 10 - protein, 305 aa, Homo sapiens
MSVDPACPQSLPCFEASDCKESSPMPVICGPEENYPSLQMSSAEMPHTETVSPLPSSMDLLIQDSPDSSTSPKGKQPTSAEKSVAKKEDKVPVKKQKTRTVFSSTQLCVLNDRFQRQKYLSLQQMQELSNILNLSYKQVKTWFQNQRMKSKRWQKNNWPKNSNGVTQKASAPTYPSLYSSYHQGCLVNPTGNLPMWSNQTWNNSTWSNQTQNIQSWSNHSWNTQTWCTQSWNNQAWNSPFYNCGEESLQSCMQFQPNSPASDLEAALEAAGEGLNVIQQTTRYFSTPQTMDLFLNYSMNMQPEDV

SEQ ID NO: 11 - DNA, 1356 bp, Mus musculus
tctatcgccttgagccgttggccttcagataggctgatttggttggtgtcttgctctttctgtgggaaggctgcggctcacttccttctgacttcttgataattttgcattagacatttaactcttctttctatgatctttccttctagacactgagttttttggttgttgcctaaaaccttttcagaaatcccttccctcgccatcacactgacatgagtgtgggtcttcctggtccccacagtttgcctagttctgaggaagcatcgaattctgggaacgcctcatcaatgcctgcagtttttcatcccgagaactattcttgcttacaagggtctgctactgagatgctctgcacagaggctgcctctcctcgcccttcctctgaagacctgcctcttcaaggcagccctgattcttctaccagtcccaaacaaaagctctcaagtcctgaggctgacaagggccctgaggaggaggagaacaaggtccttgccaggaagcagaagatgcggactgtgttctctcaggcccagctgtgtgcactcaaggacaggtttcagaagcagaagtacctcagcctccagcagatgcaagaactctcctccattctgaacctgagctataagcaggttaagacctggtttcaaaaccaaaggatgaagtgcaagcggtggcagaaaaaccagtggttgaagactagcaatggtctgattcagaagggctcagcaccagtggagtatcccagcatccattgcagctatccccagggctatctggtgaacgcatctggaagcctttccatgtggggcagccagacttggaccaacccaacttggagcagccagacctggaccaacccaacttggaacaaccagacctggaccaacccaacttggagcagccaggcctggaccgctcagtcctggaacggccagccttggaatgctgctccgctccataacttcggggaggactttctgcagccttacgtacagttgcagcaaaacttctctgccagtgatttggaggtgaatttggaagccactagggaaagccatgcgcattttagcaccccacaagccttggaattattcctgaactactctgtgactccaccaggtgaaatatgagacttacgcaacatctgggcttaaagtcagggcaaagccaggttccttccttcttccaaatattttcatattttttttaaagatttatttattcattatatgtaagtacactgtagctgtcttcagacactccagaagagggcgtcagatcttgttacgtatggttgtgagccaccatgtggttgctgggatttgaactcctgaccttcggaagagcagtcgg

SEQ ID NO: 12 - protein, 305 aa, Mus musculus
MSVGLPGPHSLPSSEEASNSGNASSMPAVFHPENYSCLQGSATEMLCTEAASPRPSSEDLPLQGSPDSSTSPKQKLSSPEADKGPEEEENKVLARKQKMRTVFSQAQLCALKDRFQKQKYLSLQQMQELSSILNLSYKQVKTWFQNQRMKCKRWQKNQWLKTSNGLIQKGSAPVEYPSIHCSYPQGYLVNASGSLSMWGSQTWTNPTWSSQTWTNPTWNNQTWTNPTWSSQAWTAQSWNGQPWNAAPLHNFGEDFLQPYVQLQQNFSASDLEVNLEATRESHAHFSTPQALELFLNYSVTPPGEI

SEQ ID NO: 13 - DNA, 19 bp, Artificial Sequence
  Description of Artificial Sequence Synthetic oligonucleotide
tgagagaggatgattctta

SEQ ID NO: 14 - DNA, 19 bp, Artificial Sequence
  Description of Artificial Sequence Synthetic oligonucleotide
ttctccgaacgtgtcacgt

SEQ ID NO: 15 - DNA, 19 bp, Artificial Sequence
  Description of Artificial Sequence Synthetic primer
caacttccagagcgaccag

SEQ ID NO: 16 - DNA, 20 bp, Artificial Sequence
  Description of Artificial Sequence Synthetic primer
acctccaggtggttgttcac

SEQ ID NO: 17 - DNA, 23 bp, Artificial Sequence
  Description of Artificial Sequence Synthetic primer
gaaggcttcttaacaccaaacaa

SEQ ID NO: 18 - DNA, 22 bp, Artificial Sequence
  Description of Artificial Sequence Synthetic primer
catttgacagaagtggtctcca

SEQ ID NO: 19 - DNA, 23 bp, Artificial Sequence
  Description of Artificial Sequence Synthetic primer
cagaagggcaaaagatcaagtat

SEQ ID NO: 20 - DNA, 20 bp, Artificial Sequence
  Description of Artificial Sequence Synthetic primer
cagtttgaatgcatgggaga

SEQ ID NO: 21 - DNA, 19 bp, Artificial Sequence
  Description of Artificial Sequence Synthetic primer
tcaacaccccagccatgta

SEQ ID NO: 22 - DNA, 18 bp, Artificial Sequence
  Description of Artificial Sequence Synthetic primer
caggtccagacgcaggat

SEQ ID NO: 23 - DNA, 24 bp, Artificial Sequence
  Description of Artificial Sequence Synthetic primer
tgggttgaaatattgggtttattt

SEQ ID NO: 24 - DNA, 23 bp, Artificial Sequence
  Description of Artificial Sequence Synthetic primer
ctaaaaccaaatatccaaccata